(19) United States
(12) Patent Application Publication
(10) Pub. No.: US 2024/0299559 A1
(43) Pub. Date: Sep. 12, 2024

(54) COMPOUNDS THAT MEDIATE PROTEIN DEGRADATION AND METHODS OF USE THEREOF

(71) Applicant: Monte Rosa Therapeutics, Inc., Boston, MA (US)

(72) Inventors: Bernhard Fasching, Basel (CH); Thomas Ryckmans, Basel (CH); Andreas Ritzén, Basel (CH)

(21) Appl. No.: 18/641,031

(22) Filed: Apr. 19, 2024

Related U.S. Application Data
(63) Continuation of application No. PCT/US2022/047410, filed on Oct. 21, 2022.
(60) Provisional application No. 63/270,919, filed on Oct. 22, 2021.

(51) Int. Cl. A61K 47/55; A61K 47/54

(52) U.S. Cl. CPC A61K 47/55; A61K 47/542; A61K 47/545

(57) ABSTRACT

Described herein, in part, are compounds that mediate the degradation of cyclin-dependent kinase 2 (CDK2), and are therefore useful in the treatment of various disorders, such as cancer.

### CROSS-REFERENCE TO RELATED APPLICATIONS

This application is a continuation of International Application Number PCT/US2022/047410, filed on Oct. 21, 2022, which claims the benefit of, and priority to, U.S. Provisional Application No. 63/270,919, filed Oct. 22, 2021, the contents of which are incorporated herein by reference.

### BACKGROUND

The ubiquitin proteasome system can be manipulated with different small molecules to trigger targeted degradation of specific proteins of interest. Promoting the targeted degradation of pathogenic proteins using small molecule degraders is emerging as a new modality in the treatment of diseases. One such modality relies on redirecting the activity of E3 ligases such as cereblon (a phenomenon known as E3 reprogramming) using low molecular weight compounds, which have been termed molecular glues to promote the poly-ubiquitination and ultimately proteasomal degradation of new protein substrates involved in the development of diseases. The molecular glues bind to both the E3 ligase and the target protein, thereby mediating an alteration of the ligase surface and enabling an interaction with the target protein.

There exists a need for therapeutics that effectively mediate the degradation of certain proteins for the treatment of diseases.

### SUMMARY

Described herein, in part, are compounds contemplated as modulators of cereblon to mediate the degradation of a protein, and are therefore are useful in the treatment of disorders, such as cancer. For example, it has been found that compounds of the present disclosure mediate the targeted degradation of the protein cyclin-dependent kinase 2 (CDK2).

In one aspect, described herein is a compound of Formula (I):

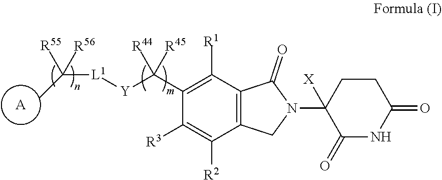

or a pharmaceutically acceptable salt thereof, wherein: X is H or deuterium; Y is NH or O; L¹ is:

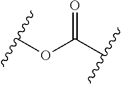 [SMILES: *OC(*)=O]

or a 5-6 membered heteroaryl; each of R¹, R², R³ is independently H, halogen, cyano, C₁₋₆ alkyl, or hydroxy; each of R⁴⁴ and R⁴⁵ is independently H or C₁₋₆ alkyl; each of R⁵⁵ and R⁵⁶ is independently H or C₁₋₆ alkyl; ring A is aryl or heteroaryl, wherein each of aryl and heteroaryl is optionally substituted with one or more occurrences of R⁴; each occurrence of R⁴ is independently halogen, cyano, —NO₂, hydroxyl, C₁₋₆ alkyl, C₂₋₆ alkynyl, C₁₋₆ alkoxy, C₁₋₆ thioalkoxy, C₃₋₁₀ cycloalkyl, 3 to 10 membered heterocyclyl, aryl, heteroaryl, or —O-heterocyclyl, wherein each of C₁₋₆ alkyl, C₂₋₆ alkynyl, C₁₋₆ alkoxy, C₁₋₆ thioalkoxy, C₃₋₁₀ cycloalkyl, 3 to 10 membered heterocyclyl, aryl, and heteroaryl is optionally substituted with one or more occurrences of R⁵; each occurrence of R⁵ is independently halogen, oxo, hydroxy, C₂₋₆ alkynyl, C₁₋₆ alkyl, C₁₋₆ alkoxy, C₁₋₆ thioalkoxy, C₃₋₁₀ cycloalkyl, 3 to 10 membered heterocyclyl, aryl, or heteroaryl, wherein C₁₋₆ alkyl is optionally substituted by one or more occurrences of halogen; n is 0, 1, 2, or 3; and m is 1 or 2; provided that the compound is not:

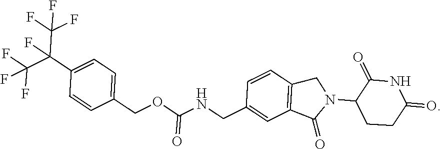 [SMILES: O=C1CCC(N2Cc3ccc(CNC(=O)OCc4ccc(C(F)(C(F)(F)F)C(F)(F)F)cc4)cc3C2=O)C(=O)N1]

In an aspect, described herein is a pharmaceutical composition comprising a compound described herein, or pharmaceutically acceptable salt thereof, and a pharmaceutically acceptable excipient.

In an aspect, described herein is a method of degrading CDK2 in a subject suffering from cancer, comprising administering to the subject an effective amount of a compound described herein, or pharmaceutically acceptable salt thereof, or a pharmaceutical composition described herein.

In an aspect, described herein is a method of treating cancer in a subject in need thereof, comprising administering to the subject a therapeutically effective amount of a compound described herein, or pharmaceutically acceptable salt thereof, or a pharmaceutical composition described herein.

In an aspect, described herein is a method of treating a solid tumor in a subject in need thereof, comprising administering to the subject a therapeutically effective amount of a compound described herein, or pharmaceutically acceptable salt thereof, or a pharmaceutical composition described herein.

In an aspect, described herein is a method of treating a liquid tumor in a subject in need thereof, comprising administering to the subject a therapeutically effective amount of a compound described herein, or pharmaceutically acceptable salt thereof, or a pharmaceutical composition described herein.

### DETAILED DESCRIPTION

The features and other details of the disclosure will now be more particularly described. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Compounds

In one aspect, described herein is a compound of Formula (I):

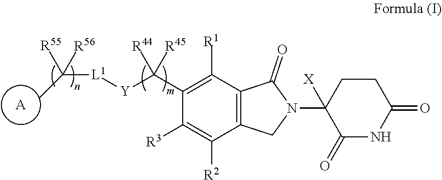

or a pharmaceutically acceptable salt thereof, wherein: X is H or deuterium; Y is NH or O; L¹ is:

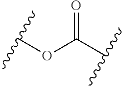 [SMILES: *OC(*)=O]

or a 5-6 membered heteroaryl; each of R¹, R², R³ is independently H, halogen, cyano, C₁₋₆ alkyl, or hydroxy; each of R⁴⁴ and R⁴⁵ is independently H or C₁₋₆ alkyl; each of R⁵⁵ and R⁵⁶ is independently H or C₁₋₄ alkyl; ring A is aryl or heteroaryl, wherein each of aryl and heteroaryl is optionally substituted with one or more occurrences of R⁴; each occurrence of R⁴ is independently halogen, cyano, —NO₂, hydroxyl, C₁₋₆ alkyl, C₂₋₆ alkynyl, C₁₋₆ alkoxy, C₁₋₆ thioalkoxy, C₃₋₁₀ cycloalkyl, 3 to 10 membered heterocyclyl, aryl, heteroaryl, or —O-heterocyclyl, wherein each of C₁₋₆ alkyl, C₂₋₆ alkynyl, C₁₋₆ alkoxy, C₁₋₆ thioalkoxy, C₃₋₁₀ cycloalkyl, 3 to 10 membered heterocyclyl, aryl, and heteroaryl is optionally substituted with one or more occurrences of R⁵; each occurrence of R⁵ is independently halogen, oxo, hydroxy, C₂₋₆ alkynyl, C₁₋₆ alkyl, C₁₋₆ alkoxy, C₁₋₆ thioalkoxy, C₃₋₁₀ cycloalkyl, 3 to 10 membered heterocyclyl, aryl, or heteroaryl, wherein C₁₋₆ alkyl is optionally substituted by one or more occurrences of halogen; n is 0, 1, 2, or 3; and m is 1 or 2; provided that the compound is not:

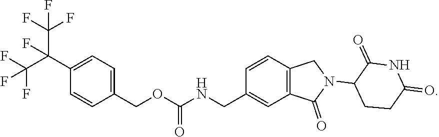 [SMILES: O=C1CCC(N2Cc3ccc(CNC(=O)OCc4ccc(C(F)(C(F)(F)F)C(F)(F)F)cc4)cc3C2=O)C(=O)N1]

In some embodiments, Y is O. In some embodiments, Y is NH.

In one aspect, described herein is a compound of Formula (I):

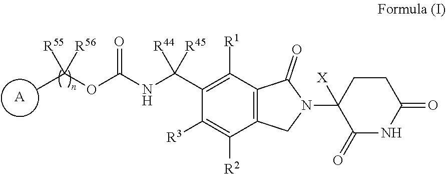

or a pharmaceutically acceptable salt thereof, wherein: X is H or deuterium; each of R¹, R², R³ is independently H, halogen, or C₁₋₆ alkyl; each of R⁴⁴ and R⁴⁵ is independently H or C₁₋₆ alkyl; each of R⁵⁵ and R⁵⁶ is independently H or C₁₋₆ alkyl; ring A is aryl or heteroaryl, wherein each of aryl and heteroaryl is optionally substituted with one or more occurrences of R⁴; each occurrence of R⁴ is independently halogen, cyano, —NO₂, hydroxyl, C₁₋₄ alkyl, C₂₋₆ alkynyl, C₁₋₆ alkoxy, C₁₋₆ thioalkoxy, C₃₋₁₀ cycloalkyl, 3 to 10 membered heterocyclyl, aryl, or heteroaryl, wherein each of C₁₋₆ alkyl, C₂₋₆ alkynyl, C₁₋₆ alkoxy, C₁₋₆ thioalkoxy, C₃₋₁₀ cycloalkyl, 3 to 10 membered heterocyclyl, aryl, and heteroaryl is optionally substituted with one or more occurrences of R⁵; each occurrence of R⁵ is independently halogen, C₁₋₆ alkyl, C₁₋₆ alkoxy, C₁₋₆ thioalkoxy, C₃₋₁₀ cycloalkyl, 3 to 10 membered heterocyclyl, aryl, or heteroaryl; each occurrence of R^{A} is independently C₁₋₆ alkyl; and n is 0, 1, 2, or 3; provided that the compound is not:

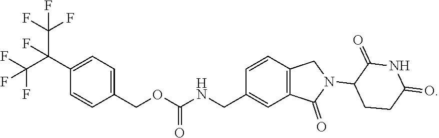 [SMILES: O=C1CCC(N2Cc3ccc(CNC(=O)OCc4ccc(C(F)(C(F)(F)F)C(F)(F)F)cc4)cc3C2=O)C(=O)N1]

In some embodiments, L¹ is

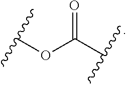 [SMILES: *OC(*)=O]

In some embodiments, L¹ is

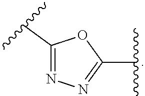 [SMILES: *c1nnc(*)o1]

In some embodiments, the compound is a compound of Formula (I-A):

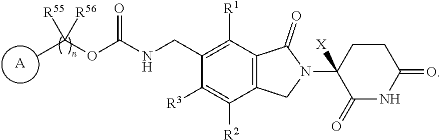

In some embodiments, the compound is a compound of Formula (I-B):

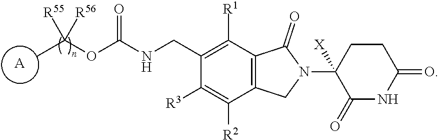

In some embodiments, the compound is a compound of Formula (I-C):

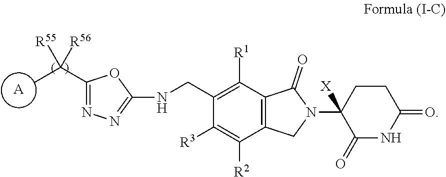

In some embodiments, the compound is a compound of Formula (I-D):

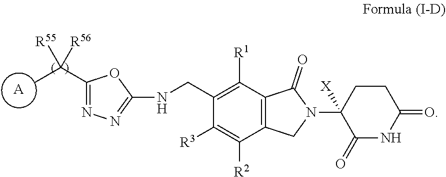

In some embodiments, X is H. In some embodiments, R¹, R², and R³ are H. In some embodiments, R⁵⁵ and R⁵⁶ are H. In some embodiments, n is 3. In some embodiments, n is 2. In some embodiments, n is 1. In some embodiments, n is 0.

In some embodiments, ring A is selected from the group consisting of:

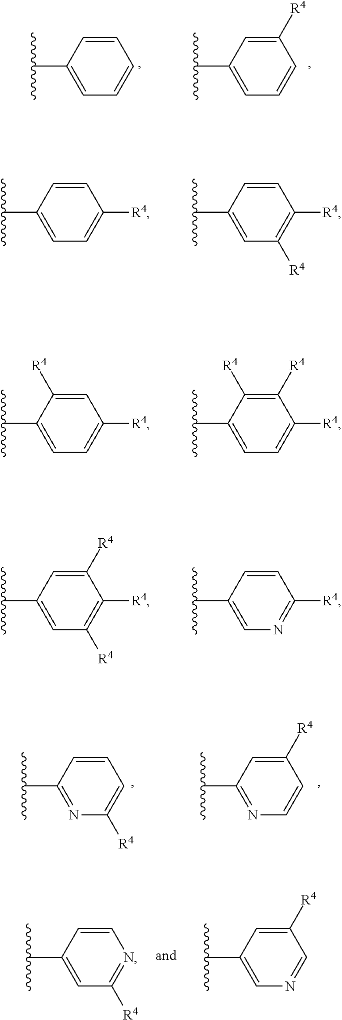

In some embodiments, ring A is C₆₋₁₀ aryl optionally substituted with one or more occurrences of R⁴. In some embodiments, ring A is 5-membered heteroaryl or 6-membered heteroaryl, wherein each of 5-membered heteroaryl and 6-membered heteroaryl optionally substituted with one or more occurrences of R⁴.

In some embodiments, ring A is selected from the group consisting of:

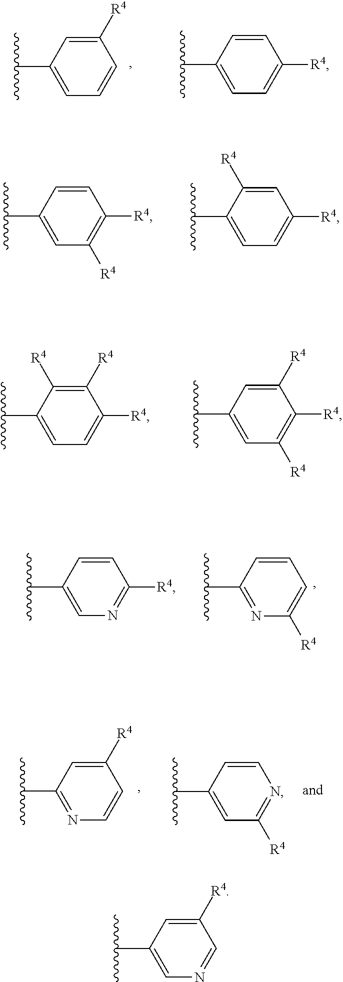

In some embodiments, ring A is

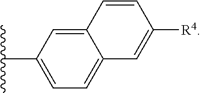

In some embodiments, R⁴ is selected from the group consisting of: flourine, chlorine, hydroxy, —CH₃, —CF₃, —OCHF₂, —OCH₃, —OCH₂CH₃, —OCF₃, —OCHF₂, —OCH₂CH₃, —OCH₂CF₃, —SCH₃, —SCF₃, —CH₂CF₃, —CH₂CH₂CH₃, —C(CH₃)(CH₃)(CH₃), —CH(CH₃)(CH₃), HC≡C(CF₃)

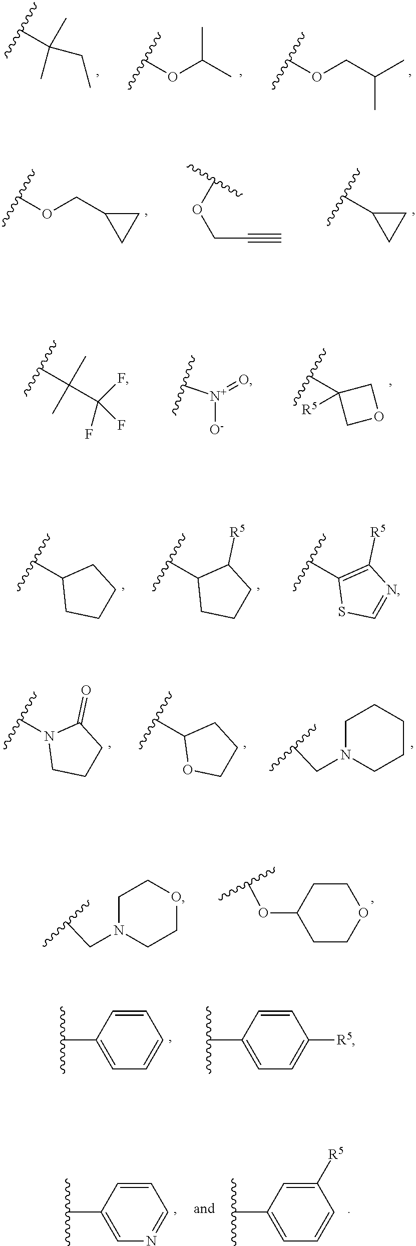

In some embodiments, R⁵ is selected from the group consisting of: flourine, chlorine, hydroxy, —CH₃, —CF₃, and —OCH₃.

In some embodiments, X is H. In some embodiments, R1, R2, and R3 are H. In some embodiments, R55 and R56 are H. In some embodiments, n is 2. In some embodiments, n is 1. In some embodiments, n is 0. In some embodiments, m is 2. In some embodiments, m is 1.

In some embodiments, the compound is a compound described in Table 1 below. Table 1 also includes the compound number of each compound in accordance with the contents of the present specification.

TABLE 1
Exemplary Compounds

| Compound No. | Structure |
|---|---|
| 1 | 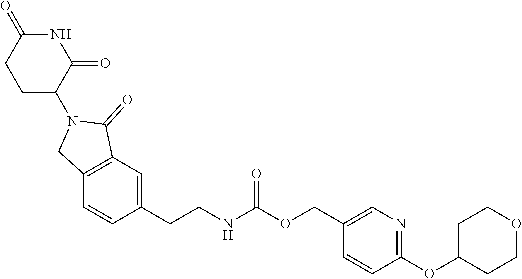 [SMILES: O=C1CCC(N2Cc3ccc(CCNC(=O)OCc4ccc(OC5CCOCC5)nc4)cc3C2=O)C(=O)N1] |
| 2 | 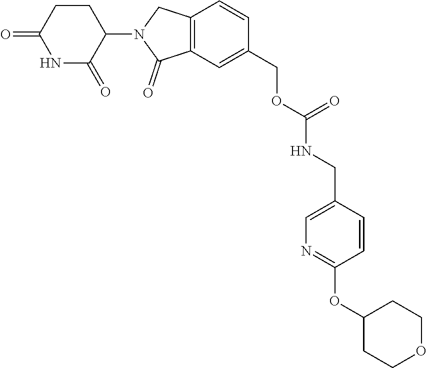 [SMILES: O=C1CCC(N2Cc3ccc(COC(=O)NCc4ccc(OC5CCOCC5)nc4)cc3C2=O)C(=O)N1] |
| 3 | 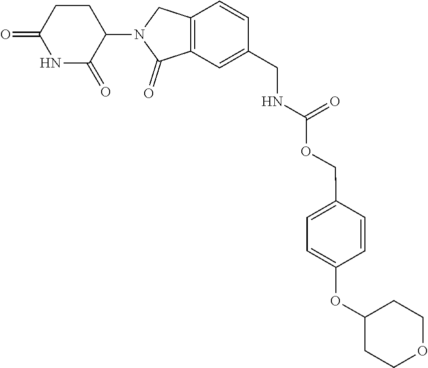 [SMILES: O=C1CCC(N2Cc3ccc(CNC(=O)OCc4ccc(OC5CCOCC5)cc4)cc3C2=O)C(=O)N1] |
| 4 | 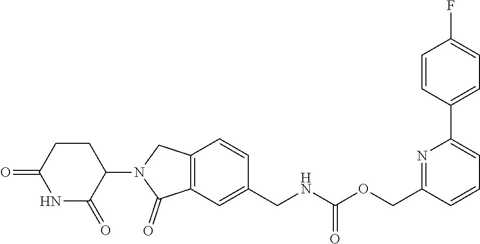 [SMILES: O=C1CCC(N2Cc3ccc(CNC(=O)OCc4cccc(-c5ccc(F)cc5)n4)cc3C2=O)C(=O)N1] |
| 5 | 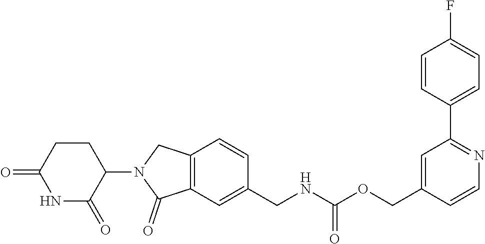 [SMILES: O=C1CCC(N2Cc3ccc(CNC(=O)OCc4ccnc(-c5ccc(F)cc5)c4)cc3C2=O)C(=O)N1] |
| 6 | 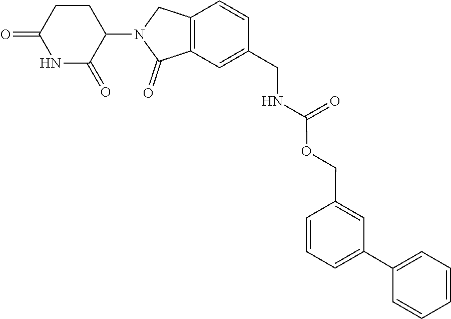 [SMILES: O=C1CCC(N2Cc3ccc(CNC(=O)OCc4cccc(-c5ccccc5)c4)cc3C2=O)C(=O)N1] |
| 7 | 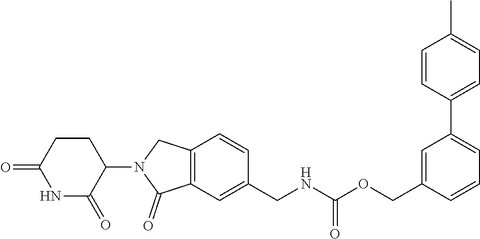 [SMILES: Cc1ccc(-c2cccc(COC(=O)NCc3ccc4c(c3)C(=O)N(C3CCC(=O)NC3=O)C4)c2)cc1] |
| 8 | 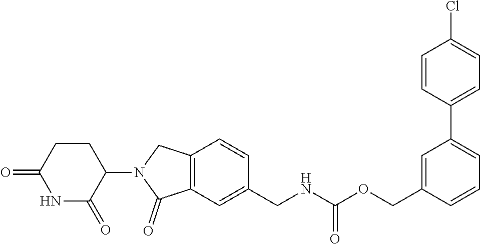 [SMILES: O=C1CCC(N2Cc3ccc(CNC(=O)OCc4cccc(-c5ccc(Cl)cc5)c4)cc3C2=O)C(=O)N1] |
| 9 | 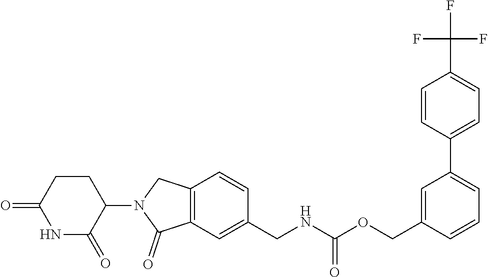 [SMILES: O=C1CCC(N2Cc3ccc(CNC(=O)OCc4cccc(-c5ccc(C(F)(F)F)cc5)c4)cc3C2=O)C(=O)N1] |
| 10 | 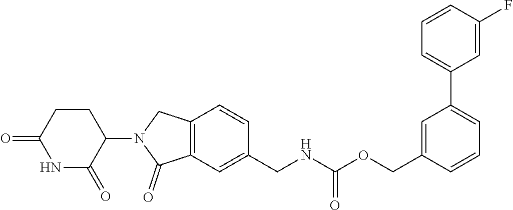 [SMILES: O=C1CCC(N2Cc3ccc(CNC(=O)OCc4cccc(-c5cccc(F)c5)c4)cc3C2=O)C(=O)N1] |
| 11 | 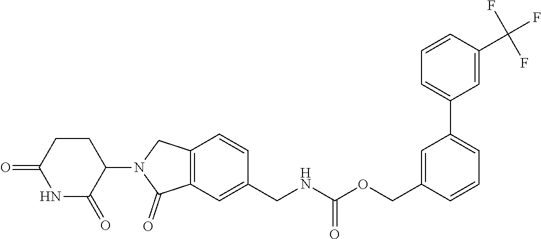 [SMILES: O=C1CCC(N2Cc3ccc(CNC(=O)OCc4cccc(-c5cccc(C(F)(F)F)c5)c4)cc3C2=O)C(=O)N1] |
| 12 | 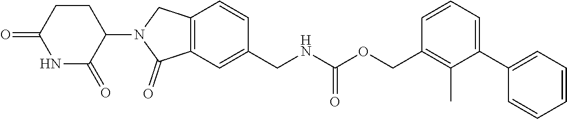 [SMILES: Cc1c(COC(=O)NCc2ccc3c(c2)C(=O)N(C2CCC(=O)NC2=O)C3)cccc1-c1ccccc1] |
| 13 | 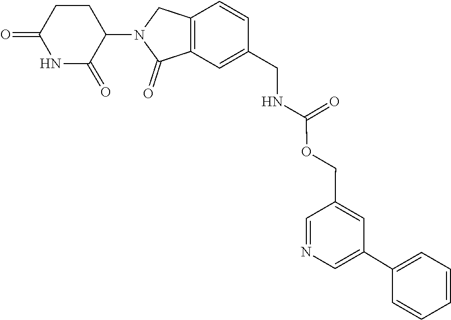 [SMILES: O=C1CCC(N2Cc3ccc(CNC(=O)OCc4cncc(-c5ccccc5)c4)cc3C2=O)C(=O)N1] |
| 14 | 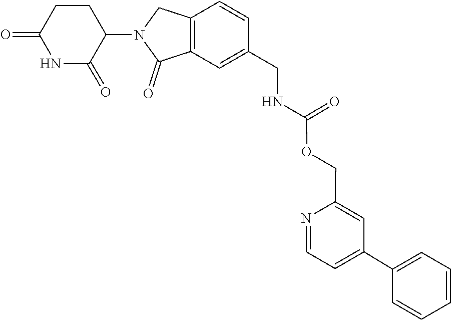 [SMILES: O=C1CCC(N2Cc3ccc(CNC(=O)OCc4cc(-c5ccccc5)ccn4)cc3C2=O)C(=O)N1] |
| 15 | 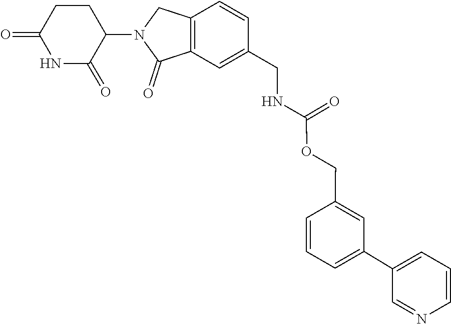 [SMILES: O=C1CCC(N2Cc3ccc(CNC(=O)OCc4cccc(-c5cccnc5)c4)cc3C2=O)C(=O)N1] |
| 16 | 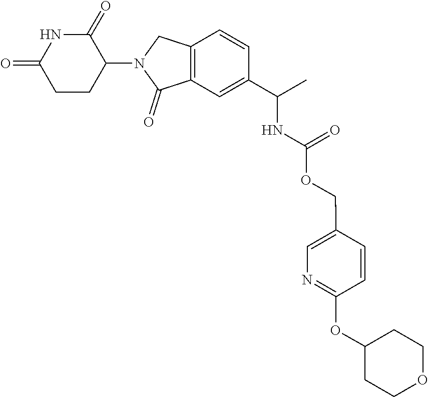 [SMILES: CC(NC(=O)OCc1ccc(OC2CCOCC2)nc1)c1ccc2c(c1)C(=O)N(C1CCC(=O)NC1=O)C2] |
| 17 | 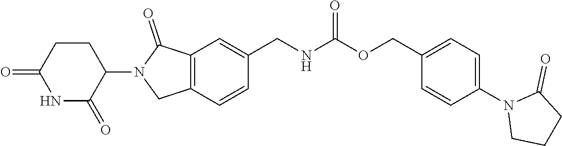 [SMILES: O=C1CCC(N2Cc3ccc(CNC(=O)OCc4ccc(N5CCCC5=O)cc4)cc3C2=O)C(=O)N1] |
| 18 | 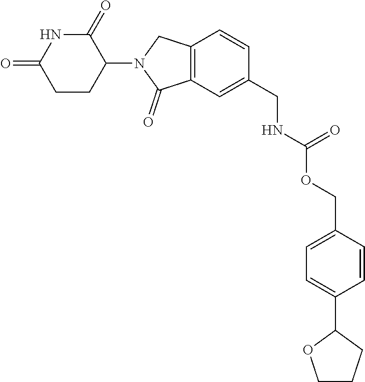 [SMILES: O=C1CCC(N2Cc3ccc(CNC(=O)OCc4ccc(C5CCCO5)cc4)cc3C2=O)C(=O)N1] |
| 19 | 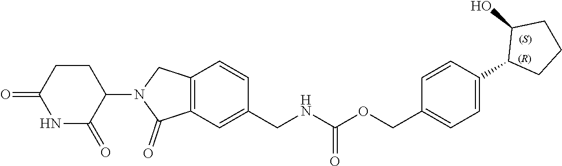 |
| 20 | 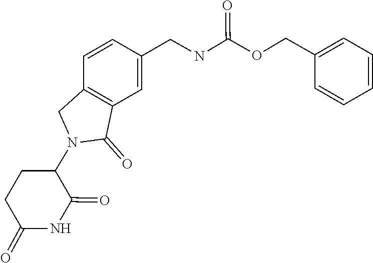 |
| 21 | 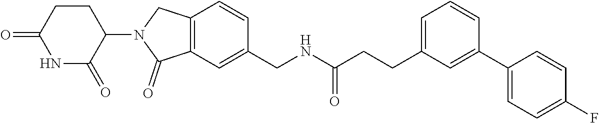 [SMILES: O=C(CCc1cccc(-c2ccc(F)cc2)c1)NCc1ccc2c(c1)C(=O)N(C1CCC(=O)NC1=O)C2] |
| 22 | 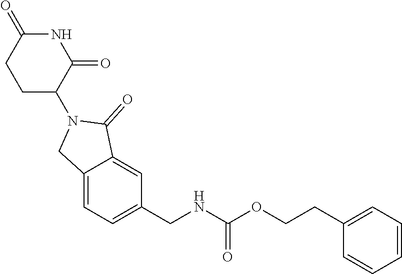 [SMILES: O=C1CCC(N2Cc3ccc(CNC(=O)OCCc4ccccc4)cc3C2=O)C(=O)N1] |
| 23 | 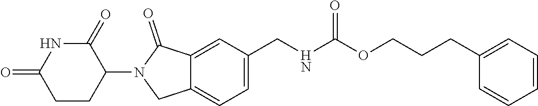 [SMILES: O=C1CCC(N2Cc3ccc(CNC(=O)OCCCc4ccccc4)cc3C2=O)C(=O)N1] |
| 24 | 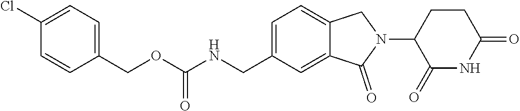 [SMILES: O=C1CCC(N2Cc3ccc(CNC(=O)OCc4ccc(Cl)cc4)cc3C2=O)C(=O)N1] |
| 25 | 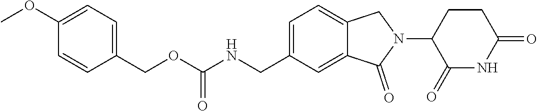 [SMILES: COc1ccc(COC(=O)NCc2ccc3c(c2)C(=O)N(C2CCC(=O)NC2=O)C3)cc1] |
| 26 | 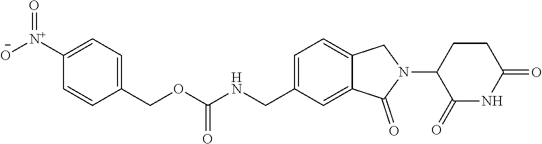 [SMILES: O=C1CCC(N2Cc3ccc(CNC(=O)OCc4ccc([N+](=O)[O-])cc4)cc3C2=O)C(=O)N1] |
| 27 | 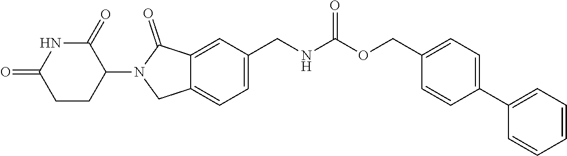 [SMILES: O=C1CCC(N2Cc3ccc(CNC(=O)OCc4ccc(-c5ccccc5)cc4)cc3C2=O)C(=O)N1] |
| 28 | 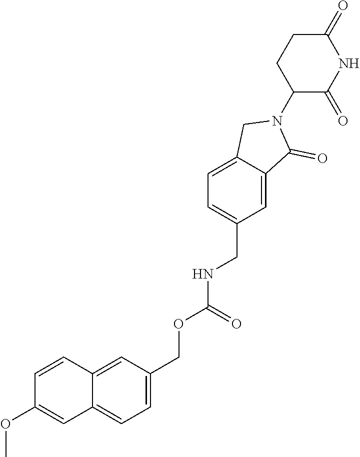 [SMILES: COc1ccc2cc(COC(=O)NCc3ccc4c(c3)C(=O)N(C3CCC(=O)NC3=O)C4)ccc2c1] |
| 29 | 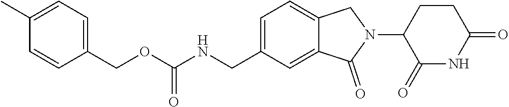 [SMILES: Cc1ccc(COC(=O)NCc2ccc3c(c2)C(=O)N(C2CCC(=O)NC2=O)C3)cc1] |
| 30 | 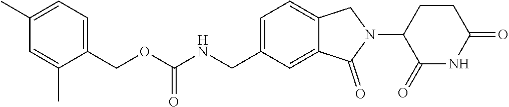 [SMILES: Cc1ccc(COC(=O)NCc2ccc3c(c2)C(=O)N(C2CCC(=O)NC2=O)C3)c(C)c1] |
| 31 | 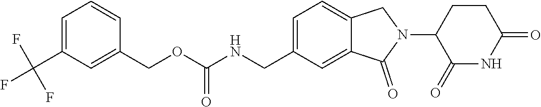 [SMILES: O=C1CCC(N2Cc3ccc(CNC(=O)OCc4cccc(C(F)(F)F)c4)cc3C2=O)C(=O)N1] |
| 32 | 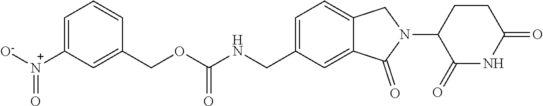 [SMILES: O=C1CCC(N2Cc3ccc(CNC(=O)OCc4cccc([N+](=O)[O-])c4)cc3C2=O)C(=O)N1] |
| 33 | 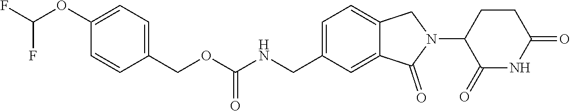 [SMILES: O=C1CCC(N2Cc3ccc(CNC(=O)OCc4ccc(OC(F)F)cc4)cc3C2=O)C(=O)N1] |
| 34 | 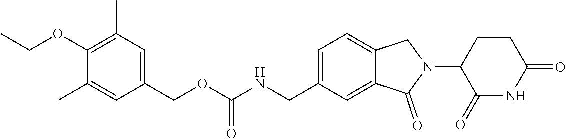 [SMILES: CCOc1c(C)cc(COC(=O)NCc2ccc3c(c2)C(=O)N(C2CCC(=O)NC2=O)C3)cc1C] |
| 35 | 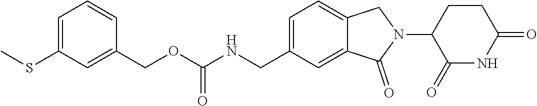 [SMILES: CSc1cccc(COC(=O)NCc2ccc3c(c2)C(=O)N(C2CCC(=O)NC2=O)C3)c1] |
| 36 | 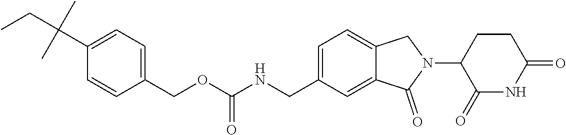 [SMILES: CCC(C)(C)c1ccc(COC(=O)NCc2ccc3c(c2)C(=O)N(C2CCC(=O)NC2=O)C3)cc1] |
| 37 | 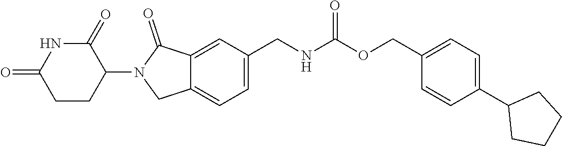 [SMILES: O=C1CCC(N2Cc3ccc(CNC(=O)OCc4ccc(C5CCCC5)cc4)cc3C2=O)C(=O)N1] |
| 38 | 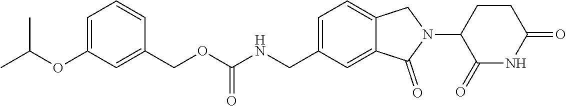 [SMILES: CC(C)Oc1cccc(COC(=O)NCc2ccc3c(c2)C(=O)N(C2CCC(=O)NC2=O)C3)c1] |
| 39 | 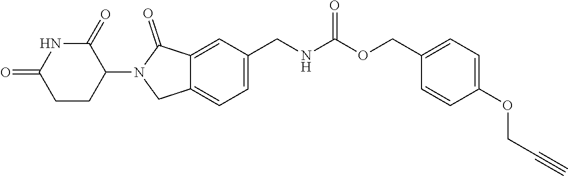 [SMILES: C#CCOc1ccc(COC(=O)NCc2ccc3c(c2)C(=O)N(C2CCC(=O)NC2=O)C3)cc1] |
| 40 | 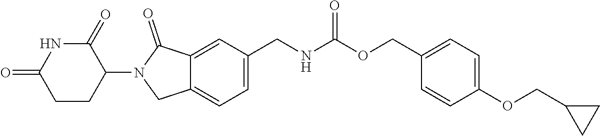 [SMILES: O=C1CCC(N2Cc3ccc(CNC(=O)OCc4ccc(OCC5CC5)cc4)cc3C2=O)C(=O)N1] |
| 41 | 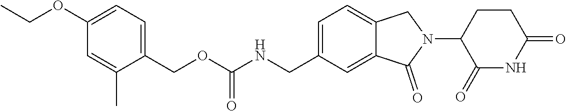 [SMILES: CCOc1ccc(COC(=O)NCc2ccc3c(c2)C(=O)N(C2CCC(=O)NC2=O)C3)c(C)c1] |
| 42 | 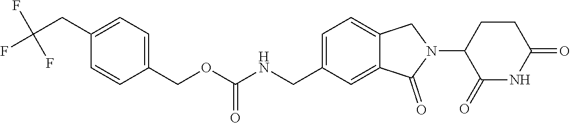 [SMILES: O=C1CCC(N2Cc3ccc(CNC(=O)OCc4ccc(CC(F)(F)F)cc4)cc3C2=O)C(=O)N1] |
| 43 | 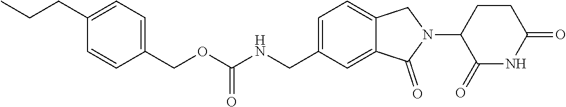 [SMILES: CCCc1ccc(COC(=O)NCc2ccc3c(c2)C(=O)N(C2CCC(=O)NC2=O)C3)cc1] |
| 44 | 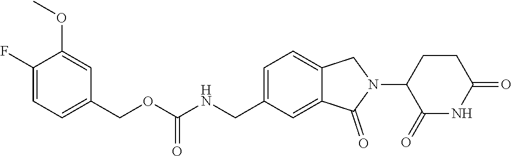 [SMILES: COc1cc(COC(=O)NCc2ccc3c(c2)C(=O)N(C2CCC(=O)NC2=O)C3)ccc1F] |
| 45 | 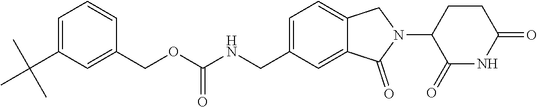 [SMILES: CC(C)(C)c1cccc(COC(=O)NCc2ccc3c(c2)C(=O)N(C2CCC(=O)NC2=O)C3)c1] |
| 46 | 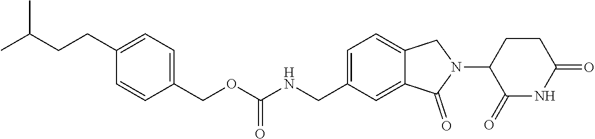 [SMILES: CC(C)CCc1ccc(COC(=O)NCc2ccc3c(c2)C(=O)N(C2CCC(=O)NC2=O)C3)cc1] |
| 47 | 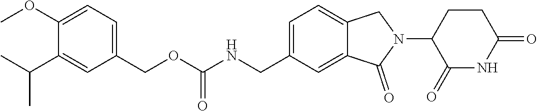 [SMILES: COc1ccc(COC(=O)NCc2ccc3c(c2)C(=O)N(C2CCC(=O)NC2=O)C3)cc1C(C)C] |
| 48 | 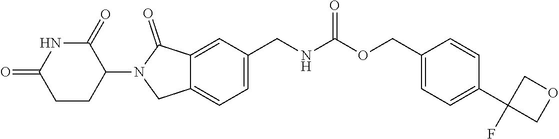 [SMILES: O=C1CCC(N2Cc3ccc(CNC(=O)OCc4ccc(C5(F)COC5)cc4)cc3C2=O)C(=O)N1] |
| 49 | 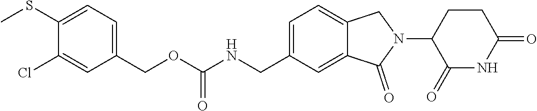 [SMILES: CSc1ccc(COC(=O)NCc2ccc3c(c2)C(=O)N(C2CCC(=O)NC2=O)C3)cc1Cl] |
| 50 | 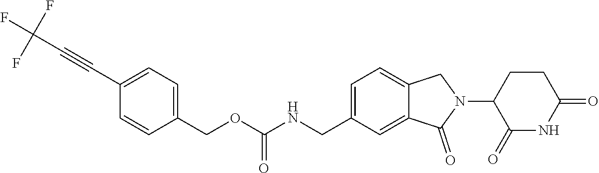 [SMILES: O=C1CCC(N2Cc3ccc(CNC(=O)OCc4ccc(C#CC(F)(F)F)cc4)cc3C2=O)C(=O)N1] |
| 51 | 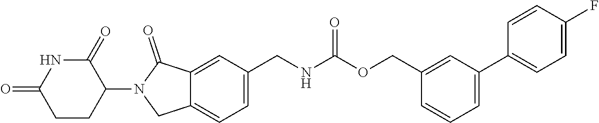 [SMILES: O=C1CCC(N2Cc3ccc(CNC(=O)OCc4cccc(-c5ccc(F)cc5)c4)cc3C2=O)C(=O)N1] |
| 52 | 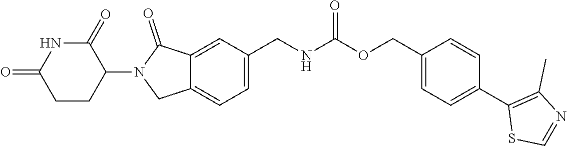 [SMILES: Cc1ncsc1-c1ccc(COC(=O)NCc2ccc3c(c2)C(=O)N(C2CCC(=O)NC2=O)C3)cc1] |
| 53 | 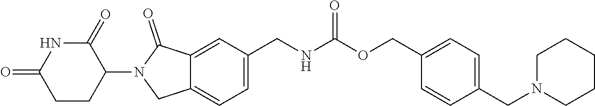 [SMILES: O=C1CCC(N2Cc3ccc(CNC(=O)OCc4ccc(CN5CCCCC5)cc4)cc3C2=O)C(=O)N1] |
| 54 | 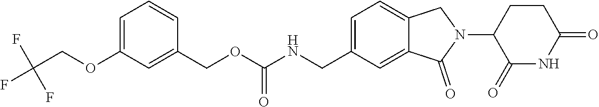 [SMILES: O=C1CCC(N2Cc3ccc(CNC(=O)OCc4cccc(OCC(F)(F)F)c4)cc3C2=O)C(=O)N1] |
| 55 | 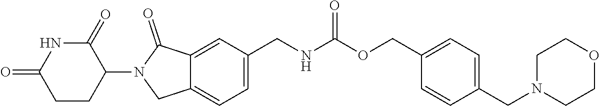 [SMILES: O=C1CCC(N2Cc3ccc(CNC(=O)OCc4ccc(CN5CCOCC5)cc4)cc3C2=O)C(=O)N1] |
| 56 | 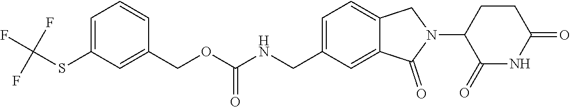 [SMILES: O=C1CCC(N2Cc3ccc(CNC(=O)OCc4cccc(SC(F)(F)F)c4)cc3C2=O)C(=O)N1] |
| 57 | 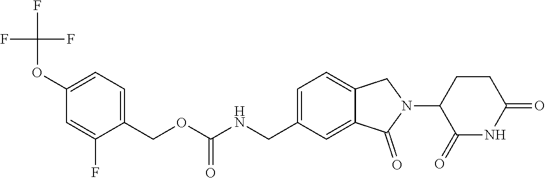 [SMILES: O=C1CCC(N2Cc3ccc(CNC(=O)OCc4ccc(OC(F)(F)F)cc4F)cc3C2=O)C(=O)N1] |
| 58 | 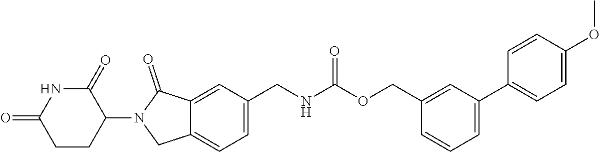 [SMILES: COc1ccc(-c2cccc(COC(=O)NCc3ccc4c(c3)C(=O)N(C3CCC(=O)NC3=O)C4)c2)cc1] |
| 59 | 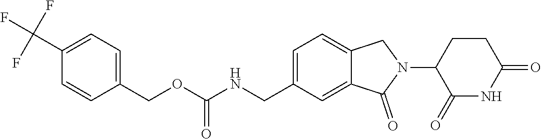 [SMILES: O=C1CCC(N2Cc3ccc(CNC(=O)OCc4ccc(C(F)(F)F)cc4)cc3C2=O)C(=O)N1] |
| 60 | 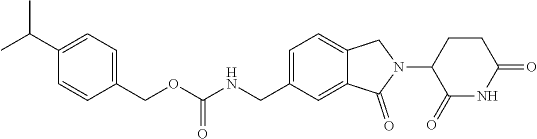 [SMILES: CC(C)c1ccc(COC(=O)NCc2ccc3c(c2)C(=O)N(C2CCC(=O)NC2=O)C3)cc1] |
| 61 | 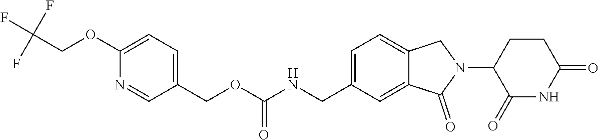 [SMILES: O=C1CCC(N2Cc3ccc(CNC(=O)OCc4ccc(OCC(F)(F)F)nc4)cc3C2=O)C(=O)N1] |
| 62 | 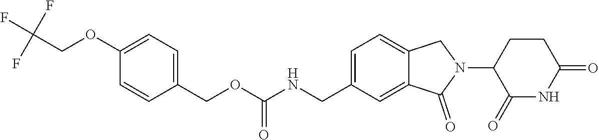 [SMILES: O=C1CCC(N2Cc3ccc(CNC(=O)OCc4ccc(OCC(F)(F)F)cc4)cc3C2=O)C(=O)N1] |
| 63 | 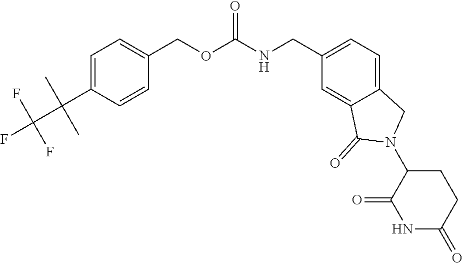 [SMILES: CC(C)(c1ccc(COC(=O)NCc2ccc3c(c2)C(=O)N(C2CCC(=O)NC2=O)C3)cc1)C(F)(F)F] |
| 64 | 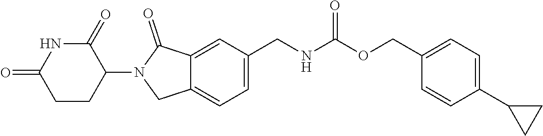 [SMILES: O=C1CCC(N2Cc3ccc(CNC(=O)OCc4ccc(C5CC5)cc4)cc3C2=O)C(=O)N1] |
| 65 | 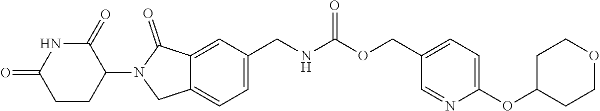 [SMILES: O=C1CCC(N2Cc3ccc(CNC(=O)OCc4ccc(OC5CCOCC5)nc4)cc3C2=O)C(=O)N1] |
| 66 | 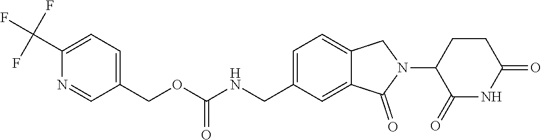 [SMILES: O=C1CCC(N2Cc3ccc(CNC(=O)OCc4ccc(C(F)(F)F)nc4)cc3C2=O)C(=O)N1] |
| 67 | 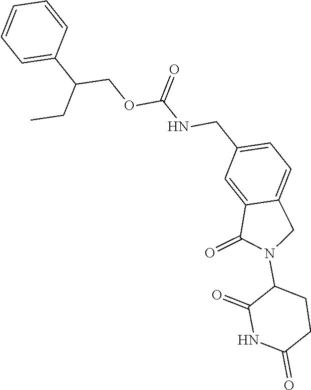 [SMILES: CCC(COC(=O)NCc1ccc2c(c1)C(=O)N(C1CCC(=O)NC1=O)C2)c1ccccc1] |
| 68 | 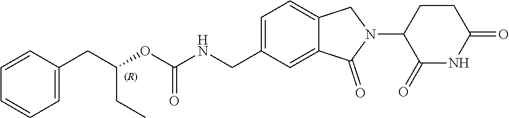 |
| 69 | 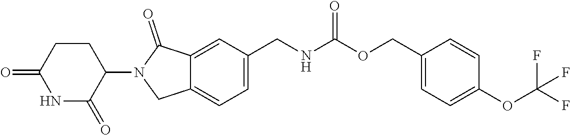 [SMILES: O=C1CCC(N2Cc3ccc(CNC(=O)OCc4ccc(OC(F)(F)F)cc4)cc3C2=O)C(=O)N1] |
| 70 | 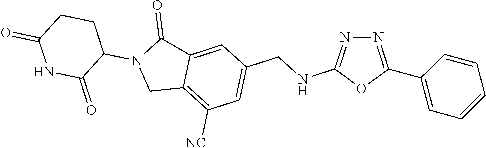 [SMILES: N#Cc1cc(CNc2nnc(-c3ccccc3)o2)cc2c1CN(C1CCC(=O)NC1=O)C2=O] |
| 71 | 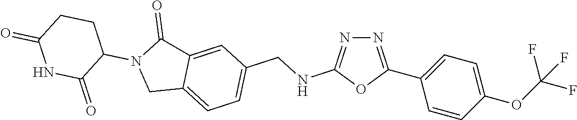 [SMILES: O=C1CCC(N2Cc3ccc(CNc4nnc(-c5ccc(OC(F)(F)F)cc5)o4)cc3C2=O)C(=O)N1] |
| 72 | 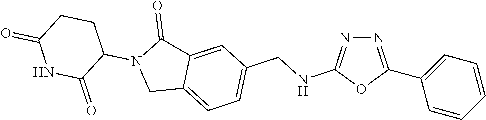 [SMILES: O=C1CCC(N2Cc3ccc(CNc4nnc(-c5ccccc5)o4)cc3C2=O)C(=O)N1] |
| 73 | 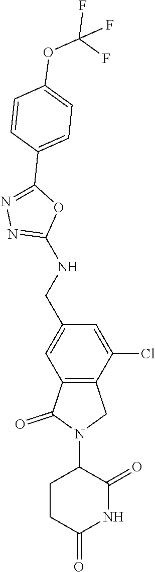 [SMILES: O=C1CCC(N2Cc3c(Cl)cc(CNc4nnc(-c5ccc(OC(F)(F)F)cc5)o4)cc3C2=O)C(=O)N1] |
| 74 | 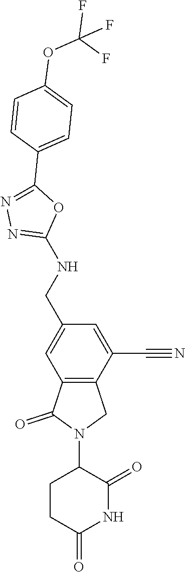 [SMILES: N#Cc1cc(CNc2nnc(-c3ccc(OC(F)(F)F)cc3)o2)cc2c1CN(C1CCC(=O)NC1=O)C2=O] |
| 75 | 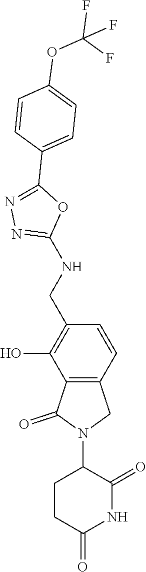 [SMILES: O=C1CCC(N2Cc3ccc(CNc4nnc(-c5ccc(OC(F)(F)F)cc5)o4)c(O)c3C2=O)C(=O)N1] |

### Pharmaceutical Compositions

In another embodiment, the present disclosure provides a pharmaceutical composition comprising a compound described herein, or pharmaceutically acceptable salt thereof, and a pharmaceutically acceptable excipient. In certain embodiments, the pharmaceutical composition comprises an effective amount of the compound. In certain embodiments, the pharmaceutical composition comprises a therapeutically effective amount of the compound.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration.

Compositions for oral administration can take the form of bulk liquid solutions or suspensions, or bulk powders. In some embodiments, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component with the remainder being various vehicles or excipients and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable excipients known in the art. As before, the active compound in such compositions is typically a minor component with the remainder being the injectable excipient and the like.

Transdermal compositions are typically formulated as a topical ointment or cream containing the active ingredient(s). When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or Formulation. All such known transdermal formulations and ingredients are included within the scope of the disclosure provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

### Methods of Treatment and Uses

Furthermore, the compounds and pharmaceutical compositions described herein are contemplated as useful in the treatment or prevention of disorders in subjects in need thereof. Compounds described herein, in one embodiment, are used to degrade CDK2 for the treatment of prevention of a disorder.

Cyclin dependent kinases, or CDKs, are a family of closely related kinases that regulate progression through the cell cycle. CDK activity is further modulated by levels of specific cyclins, for example, cyclin E1 activates cyclin dependent kinase 2, or CDK2. Tumors with CDK2 are activated by (i) the amplification of Cyclin E1 or E2 or the loss of the AMBRA1 gene and (ii) the loss of retinoblastoma. Elimination of CDK2 is contemplated to treat such disorders in patients in need thereof.

Accordingly, in one embodiment of the disclosure, a compound, or pharmaceutically acceptable salt thereof, or pharmaceutical composition described herein is administered to a subject to degrade CDK2 in the subject.

In one aspect of the disclosure, described herein is a method of treating or preventing a disorder in a subject in need thereof, comprising administering to the subject an effective amount of a compound, or pharmaceutically acceptable salt thereof, or pharmaceutical composition described herein.

In another aspect, described herein is a method of degrading CDK2 in a subject suffering from a disorder, comprising administering to the subject a therapeutically effective amount of a compound described herein, or pharmaceutically acceptable salt thereof, or pharmaceutical composition described herein. In some embodiments, the compound binds to cereblon and a CDK2 protein to induce ubiquitination and subsequent proteasomal degradation of the CDK2.

Exemplary disorders that can be treated or prevented by the methods of the present disclosure include but are not limited to, cancer of the bladder, bone, brain, breast, cervix, chest, colon, endometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands, neck, ovaries, pancreas, prostate, rectum, skin, stomach, testis, throat, or uterus. Other exemplary disorders include, but are not limited to, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer such as castration-resistant neuroendocrine prostate cancer (NEPC) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unresectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma; and blood bourne (liquid) or hematological cancers, including but not limited to leukemias, lymphomas, and myelomas, such as diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, ALK-positive large B-cell lymphoma, indolent lymphoma (for example, DLBCL, follicular lymphoma, or marginal zone lymphoma), acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AMoL), myelodysplastic syndrome (MDS), human lymphotropic virus-type 1 (HTLV-1) leukemia, mastocytosis, B-cell acute lymphoblastic leukemia, Non-Hodgkin's Lymphoma, Hodgkin's Lymphoma, and multiple myeloma (MM).

In some embodiments, the disorder is breast cancer or ovarian cancer. In some embodiments, the breast cancer is estrogen receptor positive breast cancer or triple negative breast cancer. In some embodiments, the disorder is selected from the group consisting of hormone-receptor positive breast cancer, ovarian cancer, uterine cancer, lung cancer, triple negative breast cancer, and gastric cancer.

In another aspect of the disclosure, described herein is a method of treating cancer (e.g., a cancer described herein) in a subject in need thereof, comprising administering to the subject a therapeutically effective amount of a compound described herein, or pharmaceutically acceptable salt thereof, or pharmaceutical composition described herein.

In another aspect, described herein is a method of degrading CDK2 in a subject suffering from cancer (e.g., a cancer described herein), comprising administering to the subject a therapeutically effective amount of a compound described herein, or pharmaceutically acceptable salt thereof, or pharmaceutical composition described herein.

In another aspect, described herein is a method of treating a solid tumor (e.g., a solid tumor described herein) in a subject in need thereof, comprising administering to the subject a therapeutically effective amount of a compound described herein, or pharmaceutically acceptable salt thereof, or pharmaceutical composition described herein.

In another aspect, described herein is a method of treating a liquid tumor (e.g., a liquid tumor described herein) in a subject in need thereof, comprising administering to the subject a therapeutically effective amount of a compound described herein, or pharmaceutically acceptable salt thereof, or pharmaceutical composition described herein. In some embodiments, the liquid tumor is that of a haematological cancer (e.g., a haematological cancer described herein).

### Definitions

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

When a range of values is listed, it is intended to encompass each value and sub-range within the range. For example “C1-6 alkyl” is intended to encompass, C1, C2, C3, C4, C5, C6, C1-6, C1-5, C1-4, C1-3, C1-2, C2-6, C2-5, C2-4, C2-3, C3-6, C3-5, C3-4, C4-6, C4-5, and C5-6 alkyl.

The term “alkyl” as used herein refers to a radical of a straight-chain or branched saturated hydrocarbon group. In some embodiments, an alkyl group has 1 to 12 carbon atoms (“C1-12 alkyl”). In some embodiments, an alkyl group has 1 to 10 carbon atoms (“C1-10 alkyl”). In some embodiments, an alkyl group has 1 to 9 carbon atoms (“C1-9 alkyl”). In some embodiments, an alkyl group has 1 to 8 carbon atoms (“C1-8 alkyl”). In some embodiments, an alkyl group has 1 to 7 carbon atoms (“C1-7 alkyl”). In some embodiments, an alkyl group has 1 to 6 carbon atoms (“C1-6 alkyl”, also referred to herein as “lower alkyl”). In some embodiments, an alkyl group has 1 to 5 carbon atoms (“C1-5 alkyl”). In some embodiments, an alkyl group has 1 to 4 carbon atoms (“C1-4 alkyl”). In some embodiments, an alkyl group has 1 to 3 carbon atoms (“C1-3 alkyl”). In some embodiments, an alkyl group has 1 to 2 carbon atoms (“C1-2 alkyl”). In some embodiments, an alkyl group has 1 carbon atom (“C1 alkyl”). In some embodiments, an alkyl group has 2 to 6 carbon atoms (“C2-6 alkyl”). Examples of C1-6 alkyl groups include methyl (C1), ethyl (C2), n-propyl (C3), isopropyl (C3), n-butyl (C4), tert-butyl (C4), sec-butyl (C4), iso-butyl (C4), n-pentyl (C5), 3-pentanyl (C5), amyl (C5), neopentyl (C5), 3-methyl-2-butanyl (C5), tertiary amyl (C5), and n-hexyl (C6). Additional examples of alkyl groups include n-heptyl (C7), n-octyl (C8) and the like. Common alkyl abbreviations include Me (—CH3), Et (—CH2CH3), nPr (—CH(CH3)2), nPr (—CH2CH2CH3), n-Bu (—CH2CH2CH2CH3), or i-Bu (—CH2CH(CH3)2).

The term “alkenyl” as used herein refers to a radical of a straight-chain or branched hydrocarbon group having, one or more carbon-carbon double bonds. In some embodiments, an alkenyl group has 2 to 10 carbon atoms (“C2-10 alkenyl”). In some embodiments, an alkenyl group has 2 to 9 carbon atoms (“C2-9 alkenyl”). In some embodiments, an alkenyl group has 2 to 8 carbon atoms (“C2-8 alkenyl”). In some embodiments, an alkenyl group has 2 to 7 carbon atoms (“C2-7 alkenyl”). In some embodiments, an alkenyl group has 2 to 6 carbon atoms (“C2-6 alkenyl”). In some embodiments, an alkenyl group has 2 to 5 carbon atoms (“C2-5 alkenyl”). In some embodiments, an alkenyl group has 2 to 4 carbon atoms (“C2-4 alkenyl”). In some embodiments, an alkenyl group has 2 to 3 carbon atoms (“C2-3 alkenyl”). In some embodiments, an alkenyl group has 2 carbon atoms (“C2 alkenyl”). The one or more carbon-carbon double bonds can be internal (such as in 2-butenyl) or terminal (such as in 1-butenyl). Examples of C2-4 alkenyl groups include ethenyl (C2), 1-propenyl (C3), 2-propenyl (C3), 1-butenyl (C4), 2-butenyl (C4), butadienyl (C4), and the like. Examples of C2-6 alkenyl groups include the aforementioned C2-4 alkenyl groups as well as pentenyl (C5), pentadienyl (C5), hexenyl (C6), and the like. Additional examples of alkenyl include heptenyl (C7), octenyl (C8), octatrienyl (C8), and the like.

The term “alkynyl” as used herein refers to a radical of a straight-chain or branched hydrocarbon group having one or more carbon-carbon triple bonds (e.g., 1, 2, 3, or 4 carbon-carbon triple bonds). In some embodiments, an alkynyl group has 2 to 10 carbon atoms (“C2-10 alkynyl”). In some embodiments, an alkynyl group has 2 to 9 carbon atoms (“C2-9 alkynyl”). In some embodiments, an alkynyl group has 2 to 8 carbon atoms (“C2-8 alkynyl”). In some embodiments, an alkynyl group has 2 to 7 carbon atoms (“C2-7 alkynyl”). In some embodiments, an alkynyl group has 2 to 6 carbon atoms (“C2-6 alkynyl”). In some embodiments, an alkynyl group has 2 to 5 carbon atoms (“C2-5 alkynyl”). In some embodiments, an alkynyl group has 2 to 4 carbon atoms (“C2-4 alkynyl”). In some embodiments, an alkynyl group has 2 to 3 carbon atoms (“C2-3 alkynyl”). In some embodiments, an alkynyl group has 2 carbon atoms (“C2 alkynyl”). The one or more carbon-carbon triple bonds can be internal (such as in 2-butynyl) or terminal (such as in 1-butynyl). Examples of C2-4 alkynyl groups include, without limitation, ethynyl (C2), 1-propynyl (C3), 2-propynyl (C3), 1-butynyl (C4), 2-butynyl (C4), and the like. Examples of C2-6 alkenyl groups include the aforementioned C2-4 alkynyl groups as well as pentynyl (C5), hexynyl (C6), and the like. Additional examples of alkynyl include heptynyl (C7), octynyl (C8), and the like.

The term “cycloalkyl” as used herein refers to a radical of a saturated or partially unsaturated cyclic hydrocarbon group having from 3 to 12 ring carbon atoms (“C3-12 cycloalkyl”) and zero heteroatoms in the ring system. In some embodiments, a cycloalkyl group has 3 to 10 ring carbon atoms (“C3-10 cycloalkyl”). In some embodiments, a cycloalkyl group has 3 to 8 ring carbon atoms (“C3-8 cycloalkyl”). In some embodiments, a cycloalkyl group has 3 to 6 ring carbon atoms (“C3-6 cycloalkyl”). In some embodiments, a cycloalkyl group has 3 to 6 ring carbon atoms (“C3-6 cycloalkyl”). In some embodiments, a cycloalkyl group has 5 to 10 ring carbon atoms (“C5-10 cycloalkyl”). Exemplary C3-6 cycloalkyl groups include, without limitation, cyclopropyl (C3), cyclopropenyl (C3), cyclobutyl (C4), cyclobutenyl (C4), cyclopentyl (C5), cyclopentenyl (C5), cyclohexyl (C6), cyclohexenyl (C6), cyclohexadienyl (C6), and the like. Exemplary C3-8 cycloalkyl groups include, without limitation, the aforementioned C3-6 cycloalkyl groups as well as cycloheptyl (C7), cycloheptenyl (C7), cycloheptadienyl (C7), cycloheptatrienyl (C7), cyclooctyl (C8), cyclooctenyl (C8), bicyclo[2.2.1]heptanyl (C7), bicyclo[2.2.2]octanyl (C8), and the like. Exemplary C3-10 cycloalkyl groups include, without limitation, the aforementioned C3-8 cycloalkyl groups as well as cyclononyl (C9), cyclononenyl (C9), cyclodecyl (C10), cyclodecenyl (C10), octahydro-1H-indenyl (C9), decahydronaphthalenyl (C10), spiro[4.5]decanyl (C10), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”). “Cycloalkyl” also includes ring systems wherein the cycloalkyl ring, as defined above, is fused with one or more aryl or heteroaryl groups wherein the point of attachment is on the cycloalkyl ring or the one or more aryl or heteroaryl groups, and in such instances, the number of carbons continue to designate the number of carbons in the cycloalkyl ring system.

The term “heterocyclyl” as used herein refers to a radical of a saturated or partially unsaturated 3 to 10-membered ring system having ring carbon atoms and 1 to 4 ring heteroatoms, wherein each heteroatom is independently selected from nitrogen, oxygen, sulfur, boron, phosphorus, and silicon (“3 to 10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”). Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring or the one or more aryl or heteroaryl groups, and in such instances, the number of ring members continue to designate the number of ring members in the heterocyclyl ring system.

In some embodiments, a heterocyclyl group is a 5 to 10 membered saturated or partially unsaturated ring system having ring carbon atoms and 1 to 4 ring heteroatoms, wherein each heteroatom is independently selected from nitrogen, oxygen, sulfur, boron, phosphorus, and silicon (“5 to 10 membered heterocyclyl”). In some embodiments, a heterocyclyl group is a 5 to 8 membered saturated or partially unsaturated ring system having ring carbon atoms and 1 to 4 ring heteroatoms, wherein each heteroatom is independently selected from nitrogen, oxygen, and sulfur (“5 to 8 membered heterocyclyl”). In some embodiments, a heterocyclyl group is a 5 to 6 membered saturated or partially unsaturated ring system having ring carbon atoms and 1 to 4 ring heteroatoms, wherein each heteroatom is independently selected from nitrogen, oxygen, and sulfur (“5 to 6 membered heterocyclyl”). In some embodiments, the 5 to 6 membered heterocyclyl has 1 to 3 ring heteroatoms selected from nitrogen, oxygen, and sulfur. In some embodiments, the 5 to 6 membered heterocyclyl has 1 to 2 ring heteroatoms selected from nitrogen, oxygen, and sulfur. In some embodiments, the 5 to 6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C6 aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

The term “aryl” as used herein refers to a radical of a monocyclic or polycyclic (e.g., bicyclic or tricyclic) 4n+2 aromatic ring system (e.g., having 6, 10, or 14 π electrons shared in a cyclic array) having 6 to 14 ring carbon atoms and zero heteroatoms provided in the aromatic ring system (“C6-14 aryl”). In some embodiments, an aryl group has six ring carbon atoms (“C6 aryl”; e.g., phenyl). In some embodiments, an aryl group has ten ring carbon atoms (“C10 aryl”; e.g., naphthyl such as 1-naphthyl and 2-naphthyl). In some embodiments, an aryl group has fourteen ring carbon atoms (“C14 aryl”; e.g., anthracyl). Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl.

The term “heteroaryl” as used herein refers to a radical of a 5 to 10 membered monocyclic or bicyclic 4n+2 aromatic ring system (e.g., having 6 or 10 π electrons shared in a cyclic array) having ring carbon atoms and 1 to 4 ring heteroatoms provided in the aromatic ring system, wherein each heteroatom is independently selected from nitrogen, oxygen and sulfur (“5 to 10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5 to 10 membered aromatic ring system having ring carbon atoms and 1 to 4 ring heteroatoms provided in the aromatic ring system, wherein each heteroatom is independently selected from nitrogen, oxygen, and sulfur (“5 to 10 membered heteroaryl”). In some embodiments, a heteroaryl group is a 5 to 8 membered aromatic ring system having ring carbon atoms and 1 to 4 ring heteroatoms provided in the aromatic ring system, wherein each heteroatom is independently selected from nitrogen, oxygen, and sulfur (“5 to 8 membered heteroaryl”). In some embodiments, a heteroaryl group is a monocyclic 5 to 6 membered aromatic ring system having ring carbon atoms and 1 to 4 ring heteroatoms provided in the aromatic ring system, wherein each heteroatom is independently selected from nitrogen, oxygen, and sulfur (“5 to 6 membered heteroaryl”). In some embodiments, the 5 to 6 membered heteroaryl has 1 to 3 ring heteroatoms selected from nitrogen, oxygen, and sulfur. In some embodiments, the 5 to 6 membered heteroaryl has 1 to 2 ring heteroatoms selected from nitrogen, oxygen, and sulfur. In some embodiments, the 5 to 6 membered heteroaryl has 1 ring heteroatom selected from nitrogen, oxygen, and sulfur. In some embodiments, a heteroaryl group is a monocyclic 5 membered aromatic ring system having ring carbon atoms and 1 to 4 ring heteroatoms provided in the aromatic ring system, wherein each heteroatom is independently selected from nitrogen, oxygen, and sulfur (“5-membered heteroaryl”). In some embodiments, a heteroaryl group is a monocyclic 6 membered aromatic ring system having ring carbon atoms and 1 to 4 ring heteroatoms provided in the aromatic ring system, wherein each heteroatom is independently selected from nitrogen, oxygen, and sulfur (“6-membered heteroaryl”).

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

The term “alkoxy” as used herein refers to the group —OR100 where R100 is alkyl, alkenyl, alkynyl, cycloalkyl, heterocyclyl, aryl, or heteroaryl. Exemplary alkoxy groups include methoxy, ethoxy, n-propoxy, isopropoxy, n-butoxy, tert-butoxy, sec-butoxy, n-pentoxy, n-hexoxy, and 1,2-dimethylbutoxy. Other exemplary alkoxy groups are lower alkoxy, i.e. with between 1 and 6 carbon atoms. In other examples, alkoxy groups have between 1 and 4 carbon atoms.

The term “thioalkoxy” as used herein refers to the group —SR101 where R101 is alkyl, alkenyl, alkynyl, cycloalkyl, heterocyclyl, aryl, or heteroaryl. Exemplary alkoxy groups include methoxy, ethoxy, n-propoxy, isopropoxy, n-butoxy, tert-butoxy, sec-butoxy, n-pentoxy, n-hexoxy, and 1,2-dimethylbutoxy. Other exemplary alkoxy groups are lower alkoxy, i.e. with between 1 and 6 carbon atoms. In other examples, alkoxy groups have between 1 and 4 carbon atoms.

The term “cyano” as used herein refers to the radical —CN.

The term “halogen” as used herein refers to F, Cl, Br, or I.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N+(C1-4alkyl)4 salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition. In an alternative embodiment, the present disclosure contemplates administration of the compounds described herein as a prophylactic before a subject begins to suffer from the specified disease, disorder or condition.

In general, the “effective amount” of a compound as used herein refers to an amount sufficient to elicit the desired biological response. As will be appreciated by those of ordinary skill in this art, the effective amount of a compound of the present disclosure may vary depending on such factors as the desired biological endpoint, the pharmacokinetics of the compound, the disease being treated, the mode of administration, and the age, health, and condition of the subject.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

Isomers, e.g., stereoisomers, can be isolated from mixtures by methods known to those skilled in the art, including chiral high pressure liquid chromatography (HPLC) and the formation of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, IN 1972). The present disclosure additionally encompasses compounds described herein as individual isomers substantially free of other isomers, and alternatively, as mixtures of various isomers.

The compounds provided herein can be administered as the sole active agent, or they can be administered in combination with other active agents. In some embodiments, the present invention provides a combination of a compound of the present invention and another pharmacologically active agent. Administration in combination can proceed by any technique apparent to those of skill in the art including, for example, separate, sequential, concurrent, and alternating administration.

The present disclosure, in an alternative embodiment, also embraces isotopically labeled compounds which are identical to those recited herein, except that one or more atoms are replaced by an atom having an atomic mass or mass number different from the atomic mass or mass number usually found in nature. Examples of isotopes that can be incorporated into compounds described herein include isotopes of hydrogen, carbon, nitrogen, oxygen, phosphorus, sulfur, fluorine and chlorine, such as 2H, 3H, 13C, 14C, 15N, 180, 170, 31P, 32P, 35S, 18F, and 36C1, respectively. For example, a compound of the disclosure may have one or more H atom replaced with deuterium.

### EXAMPLES

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization.

Abbreviations: CDI: 1,1′-Carbonyldiimidazole; CRBN: cereblon; DMF: N,N-dimethylformamide; eq: equivalents; DMSO: dimethyl sulfoxide; EI: electron ionization; ESI: electrospray ionization; h: hours; HPLC: high-performance liquid chromatography; LCMS: liquid chromatography mass spectrometry; MS: mass spectrometry; MTBE: tert-butyl methyl ether NMR: nuclear magnetic resonance. THF: tetrahydrofuran. LiAlH4: lithium aluminum hydride. Pd(PPH)3: bis(triphenylphosphine)palladium(II) chloride. DMEDA: dimethylethane-1,2-diamine. DCM: dichloromethane. DIEA: N,N-diisopropylethylamine.

### Example 1. Synthesis of Compound 1

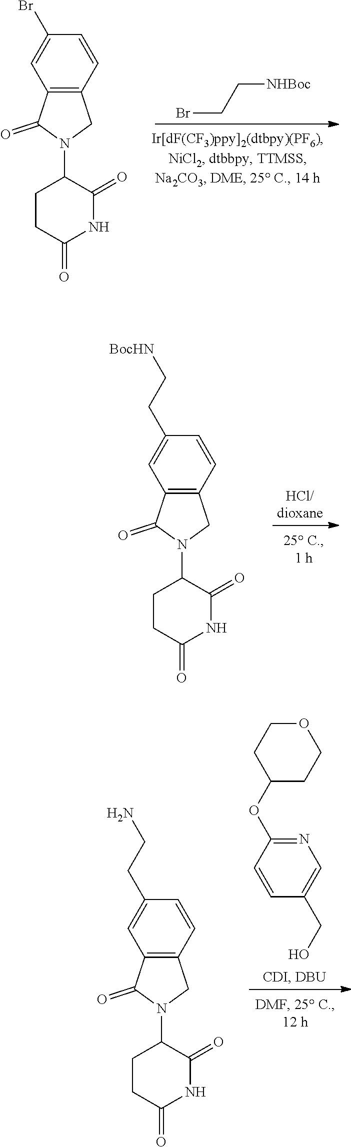 [SMILES: O=C1CCC(N2Cc3ccc(Br)cc3C2=O)C(=O)N1 ; CC(C)(C)OC(=O)NCCBr ; CC(C)(C)OC(=O)NCCc1ccc2c(c1)C(=O)N(C1CCC(=O)NC1=O)C2 ; NCCc1ccc2c(c1)C(=O)N(C1CCC(=O)NC1=O)C2 ; OCc1ccc(OC2CCOCC2)nc1]

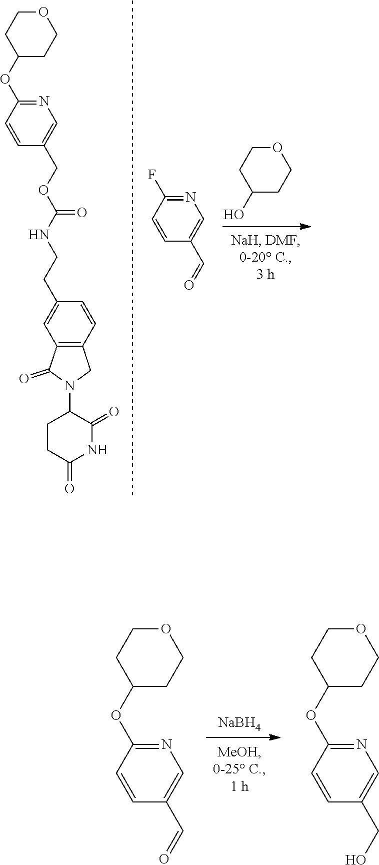

### Step 1. Synthesis of tert-butyl (2-(2-(2,6-dioxopiperidin-3-yl)-3-oxoisoindolin-5-yl)ethyl)carbamate

A mixture of 3-(6-bromo-1-oxoisoindolin-2-yl)piperidine-2,6-dione (0.500 g, 1.55 mmol, 1.00 eq), tert-butyl (2-bromoethyl)carbamate (451 mg, 2.01 mmol, 1.30 eq), bis(3,5-difluoro-2-(5-(trifluoromethyl)-2-pyridyl)phenyl)iridium(1+); 4-tert-butyl-2-(4-tert-butyl-2-pyridyl)pyridine; hexafluorophosphate (17.3 mg, 15.4 umol, 0.01 eq), nickel(II)(4,4′-di-tert-butyl-2,2′-bipyridine)dichloride (3.08 mg, 7.74 umol, 0.005 eq), bis(trimethylsilyl)silyl-trimethylsilane (385 mg, 1.55 mmol, 477 uL, 1.00 eq) and sodium carbonate (328 mg, 3.09 mmol, 2.00 eq) in dimethoxyethane (15.0 mL) was stirred under nitrogen atmosphere and irradiated with a 34 W blue LED lamp (7 cm away), with cooling fan to keep the reaction temperature at 25° C. for 14 h. The reaction mixture was concentrated to give a residue. The residue was dissolved in dimethyl sulfoxide (5.00 mL) and purified by reversed phase chromatography (C18, 80 g; condition: water/acetonitrile=1/0 to 0/1, 0.1% formic acid) to afford tert-butyl (2-(2-(2,6-dioxopiperidin-3-yl)-3-oxoisoindolin-5-yl)ethyl)carbamate (300 mg, 774 umol, 50% yield) as a white solid.

1H NMR (400 MHz, DMSO-d6) δ=10.92 (br s, 1H), 7.48 (br d, J=14.9 Hz, 2H), 7.41 (br s, 1H), 6.82 (br s, 1H), 5.14-4.94 (m, 1H), 4.44-4.31 (m, 1H), 4.30-4.19 (m, 1H), 3.12 (br s, 2H), 2.89 (br s, 1H), 2.75 (br s, 2H), 2.57 (br s, 1H), 2.35 (br d, J=11.6 Hz, 1H), 2.05-1.85 (m, 1H), 2.02-1.83 (m, 1H), 1.30 (br s, 9H).

Step 2. Synthesis of 3-(6-(2-aminoethyl)-1-oxoisoindolin-2-yl)piperidine-2,6-dione

A solution of tert-butyl (2-(2-(2,6-dioxopiperidin-3-yl)-3-oxoisoindolin-5-yl)ethyl)carbamate (100 mg, 258 umol, 1.00 eq) in hydrochloric acid/dioxane (5.00 mL) was stirred at 25° C. for 1 h. The reaction mixture was concentrated to give a residue to give 3-(6-(2-aminoethyl)-1-oxoisoindolin-2-yl)piperidine-2,6-dione (70.0 mg, crude, hydrochloride) as a white solid.

1H NMR (400 MHz, DMSO-d6) δ=11.00 (s, 1H), 8.04 (br s, 3H), 7.66 (s, 1H), 7.62-7.57 (m, 1H), 7.56-7.51 (m, 1H), 5.12 (dd, J=5.1, 13.3 Hz, 1H), 4.51-4.40 (m, 1H), 4.36-4.24 (m, 1H), 3.15-3.04 (m, 2H), 3.03-2.99 (m, 1H), 2.92 (ddd, J=5.3, 13.6, 17.5 Hz, 1H), 2.61 (br d, J=17.9 Hz, 1H), 2.47-2.36 (m, 1H), 2.09-1.93 (m, 1H).

### Step 3. Synthesis of 6-((tetrahydro-2H-pyran-4-yl)oxy) nicotinaldehyde

To a mixture of tetrahydro-2H-pyran-4-ol (3.27 g, 31.9 mmol, 3.20 mL, 2.00 eq) in dimethyl formamide (30.0 mL) was added sodium hydride (1.28 g, 31.9 mmol, 60% purity, 2.00 eq) at 0° C. The reaction mixture was stirred at 20° C. for 0.5 h. Then 6-fluoronicotinaldehyde (2.00 g, 15.9 mmol, 1.00 eq) in dimethyl formamide (20.0 mL) was added and the reaction mixture was stirred at 20° C. for 2.5 h. The reaction mixture was quenched by addition saturated ammonium chloride (500 mL) at 0° C., and then extracted with ethyl acetate (3×100 mL). The combined organic layers were washed with brine (100 mL), dried over anhydrous sodium sulfate, filtered and concentrated under reduced pressure to afford 6-((tetrahydro-2H-pyran-4-yl)oxy) nicotinaldehyde (400 mg, 1.93 mmol, 12% yield) as yellow oil.

1H NMR (400 MHz, DMSO-d6) δ=9.96 (s, 1H), 8.75 (d, J=2.0 Hz, 1H), 8.12 (dd, J=2.4, 8.6 Hz, 1H), 6.97 (d, J=8.6 Hz, 1H), 5.34 (tt, J=4.4, 9.2 Hz, 1H), 3.89-3.85 (m, 2H), 3.53-3.48 (m, 2H), 2.06-2.01 (m, 2H), 1.70-1.63 (m, 2H).

### Step 4. Synthesis of (6-((tetrahydro-2H-pyran-4-yl)oxy)pyridin-3-yl)methanol

To a mixture of 6-((tetrahydro-2H-pyran-4-yl)oxy) nicotinaldehyde (400 mg, 1.93 mmol, 1.00 eq) in methanol (4.00 mL) was added sodium borohydride (365 mg, 9.65 mmol, 5.00 eq) at 0° C. The reaction mixture was stirred at 25° C. for 1 h. The reaction mixture was quenched by addition saturated ammonium chloride (50.0 mL) at 0° C. and extracted with ethyl acetate (3×20.0 mL). The combined organic layers were washed with brine (50.0 mL), dried over anhydrous sodium sulfate, filtered and concentrated under reduced pressure to give (6-((tetrahydro-2H-pyran-4-yl)oxy)pyridin-3-yl)methanol (400 mg, crude) as an off-white solid.

1H NMR (400 MHz, DMSO-d6) δ=8.06 (d, J=2.0 Hz, 1H), 7.64 (dd, J=2.4, 8.4 Hz, 1H), 6.76 (d, J=8.4 Hz, 1H), 5.20-5.16 (m, 1H), 5.15-5.13 (m, 1H), 4.42 (d, J=5.6 Hz, 2H), 3.86 (td, J=4.2, 11.6 Hz, 2H), 3.55-3.43 (m, 2H), 2.04-1.94 (m, 2H), 1.61 (dtd, J=4.0, 9.4, 13.2 Hz, 2H).

### Step 5. Synthesis of (6-((tetrahydro-2H-pyran-4-yl)oxy)pyridin-3-yl)methyl (2-(2-(2,6-dioxopiperidin-3-yl)-3-oxoisoindolin-5-yl)ethyl)carbamate

To a mixture of (6-((tetrahydro-2H-pyran-4-yl)oxy)pyridin-3-yl)methanol (21.8 mg, 104 umol, 1.00 eq) in dimethyl formamide (0.500 mL) was added 1,1′-carbonyldiimidazole (50.7 mg, 313 umol, 3.00 eq) and 1,8-diazabicyclo[5.4.0]undec-7-ene (47.6 mg, 313 umol, 47.2 uL, 3.00 eq). The reaction mixture was stirred at 25° C. for 1.5 h. Then 3-(6-(2-aminoethyl)-1-oxoisoindolin-2-yl)piperidine-2,6-dione (30.0 mg, 104 umol, 1.00 eq) in dimethyl formamide (0.500 mL) was added to the mixture. The mixture was stirred at 25° C. for 10.5 h. The reaction mixture was added to formic acid (2.00 mL) and filtered. The filtrate was purified by Prep-HPLC (column: Shim-pack C18 150*25*10 um; mobile phase: [water (formic acid)-acetonitrile]; B %: 25%-55%, 10 min) and lyophilized to afford (6-((tetrahydro-2H-pyran-4-yl)oxy)pyridin-3-yl)methyl (2-(2-(2,6-dioxopiperidin-3-yl)-3-oxoisoindolin-5-yl)ethyl)carbamate (9.48 mg, 17.2 umol, 16% yield, 95% purity, formic acid) as a white solid.

1H NMR (400 MHz, DMSO-d6) δ=10.99 (br s, 1H), 8.47 (s, 1H), 8.12 (d, J=2.2 Hz, 1H), 7.65 (dd, J=2.2, 8.6 Hz, 1H), 7.56 (s, 1H), 7.52-7.47 (m, 1H), 7.46-7.41 (m, 1H), 7.32 (t, J=5.6 Hz, 1H), 6.78 (d, J=8.4 Hz, 1H), 5.22-5.07 (m, 2H), 4.92 (s, 2H), 4.45-4.37 (m, 1H), 4.33-4.25 (m, 1H), 3.85 (td, J=4.4, 11.6 Hz, 2H), 3.51-3.45 (m, 2H), 3.28-3.22 (m, 2H), 2.97-2.86 (m, 1H), 2.82 (br t, J=6.8 Hz, 2H), 2.61 (br d, J=2.6 Hz, 1H), 2.43-2.36 (m, 1H), 2.04-1.95 (m, 3H), 1.66-1.55 (m, 2H).

### Example 2. Synthesis of Compound 2

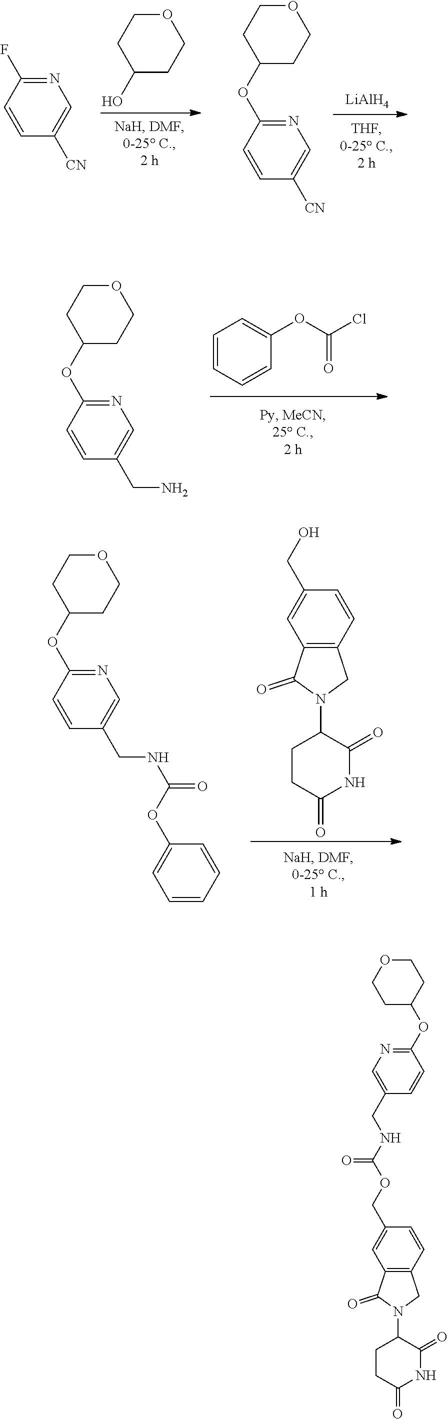

### Step 1. Synthesis of 6-((tetrahydro-2H-pyran-4-yl)oxy)nicotinonitrile

To a mixture of sodium hydride (197 mg, 4.91 mmol, 60% purity, 1.20 eq) in tetrahydrofuran (3.00 mL) was added tetrahydro-2H-pyran-4-ol (502 mg, 4.91 mmol, 492 uL, 1.20 eq) at 0° C. After 1 h, 6-fluoronicotinonitrile (500 mg, 4.10 mmol, 1.00 eq) was added, then the mixture was stirred at 25° C. for 1 h. The reaction mixture was concentrated under reduced pressure to give a residue. The residue was purified by column chromatography (SiO₂, petroleum ether/ethyl acetate=1/0 to 0/1) to afford 6-((tetrahydro-2H-pyran-4-yl)oxy)nicotinonitrile (500 mg, 2.45 mmol, 60% yield) as a yellow solid.

1H NMR (400 MHz, CDCl3) δ=8.47 (d, J=2.2 Hz, 1H), 7.80 (dd, J=2.2, 8.7 Hz, 1H), 6.82 (d, J=8.8 Hz, 1H), 5.33 (tt, J=4.2, 8.6 Hz, 1H), 4.04-3.98 (m, 2H), 3.66-3.60 (m, 2H), 2.12-2.06 (m, 2H), 1.82 (qd, J=4.6, 13.2 Hz, 2H).

### Step 2. Synthesis of (6-((tetrahydro-2H-pyran-4-yl)oxy)pyridin-3-yl)methanamine

To a mixture of 6-((tetrahydro-2H-pyran-4-yl)oxy)nicotinonitrile (500 mg, 2.45 mmol, 1.00 eq) in tetrahydrofuran (5.00 mL) was added lithium aluminum hydride (102 mg, 2.69 mmol, 1.10 eq) at 0° C. Then the mixture was stirred at 25° C. for 2 h. The reaction mixture was quenched by addition water (0.1 mL), 10% sodium hydroxide solution (0.1 mL) and water (0.3 mL). Then the reaction mixture was filtered and concentrated under reduced pressure to afford (6-((tetrahydro-2H-pyran-4-yl)oxy)pyridin-3-yl)methanamine (0.800 g, crude) as a yellow solid.

1H NMR (400 MHz, CDCl3) δ=8.30-8.24 (m, 1H), 8.06-8.00 (m, 1H), 6.69-6.63 (m, 1H), 5.46-4.96 (m, 2H), 4.63 (s, 1H), 3.91 (br dd, J=4.2, 7.5 Hz, 2H), 3.56-3.52 (m, 2H), 1.99 (br dd, J=4.0, 5.0 Hz, 2H), 1.74-1.70 (m, 2H), 1.59-1.35 (m, 2H).

### Step 3. Synthesis of phenyl ((6-((tetrahydro-2H-pyran-4-yl)oxy)pyridin-3-yl)methyl)carbamate

To a mixture of (6-((tetrahydro-2H-pyran-4-yl)oxy)pyridin-3-yl)methanamine (800 mg, 3.84 mmol, 1.00 eq) in acetonitrile (10.0 mL) was added phenyl carbonochloridate (902 mg, 5.76 mmol, 722 uL, 1.50 eq) and pyridine (608 mg, 7.68 mmol, 620 uL, 2.00 eq). Then the mixture was stirred at 25° C. for 2 h. The reaction mixture was filtered and concentrated under reduced pressure to give a residue. The residue was purified by Prep-HPLC (formic acid; column: Shim-pack C18 150×25×10 um; mobile phase: [water(0.225% formic acid)-acetonitrile]; B %: 30%-60%, 10 min) to afford phenyl ((6-((tetrahydro-2H-pyran-4-yl)oxy)pyridin-3-yl)methyl)carbamate (100 mg, 305 umol, 8% yield) as a yellow solid.

1H NMR (400 MHz, CDCl3) δ=8.11 (d, J=1.8 Hz, 1H), 7.64 (dd, J=2.4, 8.5 Hz, 1H), 7.40-7.36 (m, 2H), 7.26-7.22 (m, 1H), 7.15 (br d, J=7.8 Hz, 2H), 6.75 (d, J=8.6 Hz, 1H), 5.26 (td, J=4.2, 8.5 Hz, 1H), 4.40 (d, J=6.0 Hz, 2H), 4.03-3.98 (m, 2H), 3.67-3.62 (m, 2H), 2.14-2.04 (m, 3H), 1.84-1.79 (m, 2H).

### Step 4. Synthesis of (2-(2,6-dioxopiperidin-3-yl)-3-oxoisoindolin-5-yl)methyl((6-((tetrahydro-2H-pyran-4-yl)oxy)pyridin-3-yl)methyl)carbamate

To a mixture of 3-(6-(hydroxymethyl)-1-oxoisoindolin-2-yl)piperidine-2,6-dione (50.0 mg, 182 umol, 1.00 eq) and phenyl ((6-((tetrahydro-2H-pyran-4-yl)oxy)pyridin-3-yl)methyl)carbamate (59.9 mg, 182 umol, 1.00 eq) in N,N-dimethylformamide (1.00 mL) was added sodium hydride (14.6 mg, 365 umol, 60% purity, 2.00 eq) at 0° C. Then the mixture was stirred at 25° C. for 1 h. The reaction mixture was filtered. The residue was purified by Prep-HPLC (formic acid; column: Unisil 3-100 C18 Ultra 150×50 mm×3 um; mobile phase: [water(formic acid)-acetonitrile]; B %: 15%-45%, 10 min) to afford (2-(2,6-dioxopiperidin-3-yl)-3-oxoisoindolin-5-yl)methyl((6-((tetrahydro-2H-pyran-4-yl)oxy)pyridin-3-yl)methyl)carbamate (24.48 mg, 43.70 umol, 24% yield, 99% purity, formate) as a white solid.

1H NMR (400 MHz, DMSO-d6) δ=11.00 (br s, 1H), 8.27 (s, 1H), 8.03 (d, J=1.6 Hz, 1H), 7.85 (br t, J=6.0 Hz, 1H), 7.72 (s, 1H), 7.65-7.56 (m, 3H), 6.76 (d, J=8.4 Hz, 1H), 5.18-5.09 (m, 4H), 4.50-4.43 (m, 1H), 4.37-4.30 (m, 1H), 4.14 (d, J=6.0 Hz, 2H), 3.85 (td, J=4.2, 11.6 Hz, 2H), 3.49 (br s, 2H), 2.98-2.85 (m, 1H), 2.62 (br s, 1H), 2.45-2.34 (m, 1H), 2.03-1.95 (m, 3H), 1.65-1.56 (m, 2H).

### Example 3. Synthesis of Compound 3

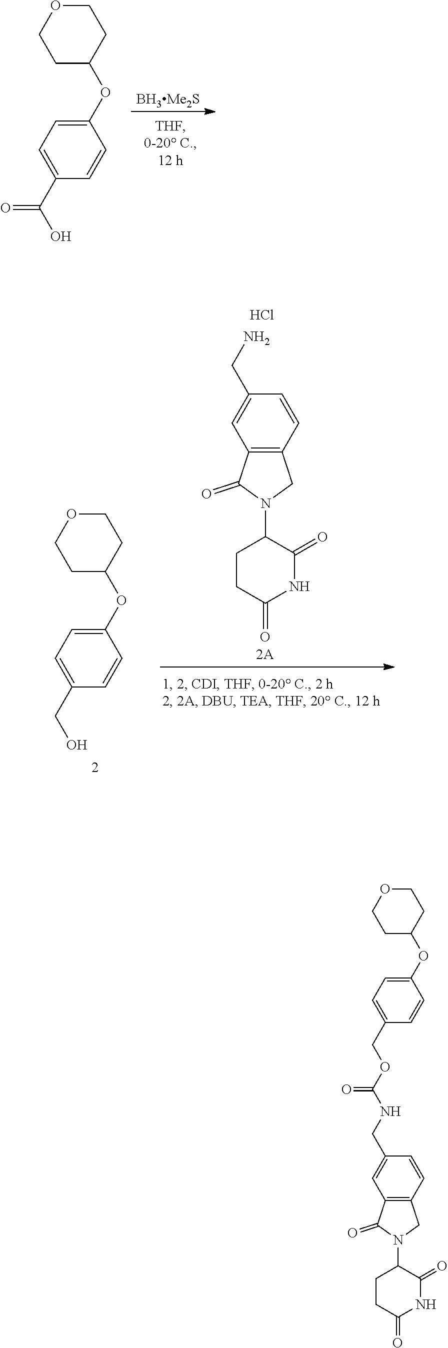

### Step 1. Synthesis of (4-((tetrahydro-2H-pyran-4-yl)oxy)phenyl)methanol

To a solution of 4-((tetrahydro-2H-pyran-4-yl)oxy)benzoic acid (100 mg, 450 umol, 1.00 eq) in tetrahydrofuran (2.00 mL) was added borane dimethyl sulfide complex (10.0 M, 90.0 uL, 2.00 eq) at 0° C. The reaction mixture was stirred at 20° C. for 12 h. The reaction mixture was quenched with methanol (10 mL) and then concentrated under reduced pressure to afford (4-((tetrahydro-2H-pyran-4-yl)oxy)phenyl)methanol (80.0 mg, 384 umol, 85% yield) as colorless oil.

1H NMR (400 MHz, DMSO-d6) δ=7.21 (d, J=8.4 Hz, 2H), 6.94-6.87 (m, 2H), 5.05 (br t, J=5.4 Hz, 1H), 4.57-4.48 (m, 1H), 4.40 (d, J=4.9 Hz, 2H), 3.83 (td, J=4.4, 11.6 Hz, 2H), 3.49-3.44 (m, 2H), 1.98-1.91 (m, 2H), 1.61-1.50 (m, 2H).

### Step 2. Synthesis of 4-((tetrahydro-2H-pyran-4-yl)oxy)benzyl((2-(2,6-dioxopiperidin-3-yl)-3-oxoisoindolin-5-yl)methyl)carbamate

To a solution of (4-((tetrahydro-2H-pyran-4-yl)oxy)phenyl)methanol (50.0 mg, 240 umol, 1.00 eq) in tetrahydrofuran (0.500 mL) was added di(1H-imidazol-1-yl)methanone (77.9 mg, 480 umol, 2.00 eq). The mixture was stirred at 20° C. for 2 h. The resulting solution was added to a mixture of 3-(6-(aminomethyl)-1-oxoisoindolin-2-yl)piperidine-2,6-dione (74.4 mg, 240 umol, 1.00 eq, hydrochloride), 2,3,4,6,7,8,9,10-octahydropyrimido[1,2-a]azepine (36.6 mg, 240 umol, 36.2 uL, 1.00 eq) and triethylamine (24.3 mg, 240 umol, 33.4 uL, 1.00 eq) in tetrahydrofuran (0.500 mL). The reaction mixture was stirred at 20° C. for 12 h. The reaction mixture was concentrated under reduced pressure to give a residue. The residue was purified by Prep-HPLC(column: Unisil 3-100 C18 Ultra 150*50 mm*3 um; mobile phase: [water(formic acid)-acetonitrile]; B %: 25%-55%, 10 min) and lyophilized to afford 4-((tetrahydro-2H-pyran-4-yl)oxy)benzyl((2-(2,6-dioxopiperidin-3-yl)-3-oxoisoindolin-5-yl)methyl)carbamate (29.20 mg, 56.96 umol, 24% yield, 99% purity) as an off-white solid.

1H NMR (400 MHz, DMSO-d6) δ=10.99 (br s, 1H), 7.86 (t, J=6.1 Hz, 1H), 7.62 (s, 1H), 7.58-7.45 (m, 2H), 7.28 (d, J=8.4 Hz, 2H), 6.96 (d, J=8.6 Hz, 2H), 5.11 (dd, J=5.0, 13.2 Hz, 1H), 4.96 (s, 2H), 4.61-4.52 (m, 1H), 4.48-4.39 (m, 1H), 4.35-4.23 (m, 3H), 3.83 (td, J=4.4, 11.5 Hz, 2H), 3.50-3.45 (m, 2H), 2.98-2.84 (m, 1H), 2.63-2.57 (m, 1H), 2.46-2.35 (m, 1H), 2.05-1.90 (m, 3H), 1.63-1.49 (m, 2H). MS (ESI) m/z 508.1 [M+H]+.

### Example 4. Synthesis of Compound 4

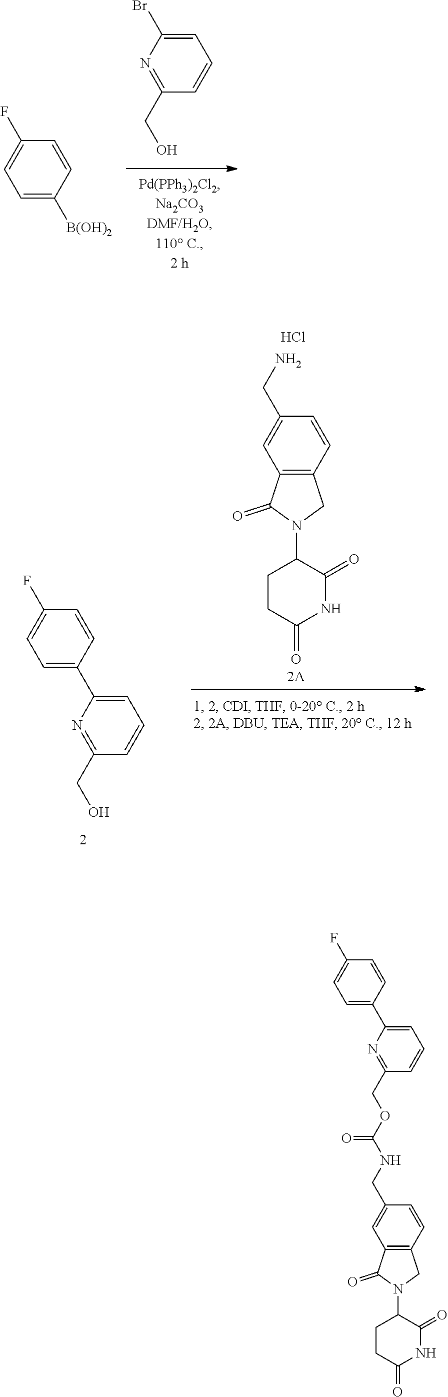 [SMILES: OB(O)c1ccc(F)cc1 ; OCc1cccc(Br)n1 ; Cl ; OCc1cccc(-c2ccc(F)cc2)n1 ; NCc1ccc2c(c1)C(=O)N(C1CCC(=O)NC1=O)C2 ; O=C1CCC(N2Cc3ccc(CNC(=O)OCc4cccc(-c5ccc(F)cc5)n4)cc3C2=O)C(=O)N1]

### Step 1. Synthesis of (6-(4-fluorophenyl)pyridin-2-yl)methanol

To a solution of (6-bromopyridin-2-yl)methanol (500 mg, 2.66 mmol, 1.00 eq) in dimethylformamide (5.00 mL) and water (0.625 mL) were added sodium carbonate (705 mg, 6.65 mmol, 2.50 eq), bis(triphenylphosphine)palladium(II) chloride (373 mg, 532 umol, 0.200 eq) and (4-fluorophenyl)boronic acid (447 mg, 3.19 mmol, 1.20 eq). The reaction mixture was stirred at 110° C. for 2 h. The reaction mixture was filtered and the filtrate was added water (70 mL), then extracted with ethyl acetate (3×20 mL). The combined organic layers were washed with brine (40 mL), dried over anhydrous sodium sulfate, filtered and concentrated under reduced pressure to give a residue. The residue was purified by column chromatography (SiO₂, petroleum ether/ethyl acetate=1/0 to 3/1) to afford (6-(4-fluorophenyl)pyridin-2-yl)methanol(130 mg, 640 umol, 24% yield) as yellow oil.

1H NMR (400 MHz, DMSO-d6) δ=8.22-8.09 (m, 2H), 7.92-7.85 (m, 1H), 7.85-7.78 (m, 1H), 7.43 (d, J=7.6 Hz, 1H), 7.38-7.26 (m, 2H), 5.44 (t, J=5.8 Hz, 1H), 4.63 (d, J=5.9 Hz, 2H).

### Step 2. Synthesis of (6-(4-fluorophenyl)pyridin-2-yl)methyl((2-(2,6-dioxopiperidin-3-yl)-3-oxoisoindolin-5-yl)methyl)carbamate

To a solution of (6-(4-fluorophenyl)pyridin-2-yl)methanol (50.0 mg, 246 umol, 1.00 eq) in tetrahydrofuran (0.500 mL) was added di(1H-imidazol-1-yl)methanone (79.8 mg, 492 umol, 2.00 eq). The mixture was stirred at 20° C. for 2 h. The resulting solution was added to a mixture of 3-(6-(aminomethyl)-1-oxoisoindolin-2-yl)piperidine-2,6-dione (76.2 mg, 246 umol, 1.00 eq, hydrochloride), 2,3,4,6,7,8,9,10-octahydropyrimido[1,2-a]azepine (37.5 mg, 246 umol, 37.1 uL, 1.00 eq) and triethylamine (24.9 mg, 246 umol, 34.3 uL, 1.00 eq) in tetrahydrofuran (0.500 mL). The reaction mixture was stirred at 20° C. for 12 h. The reaction mixture was concentrated under reduced pressure to give a residue. The residue was purified by Prep-HPLC(column: Unisil 3-100 C18 Ultra 150*50 mm*3 um; mobile phase: [water(formic acid)-acetonitrile]; B %: 30%-60%, 10 min) and lyophilized to give a crude product. The crude product was purified by Prep-HPLC(column: YMC Triart 30*150 mm*7 um; mobile phase: [water(hydrochloric acid)-acetonitrile]; B %: 32%-52%, 9 min) and lyophilized to afford (6-(4-fluorophenyl)pyridin-2-yl)methyl((2-(2,6-dioxopiperidin-3-yl)-3-oxoisoindolin-5-yl)methyl)carbamate (25.04 mg, 49.33 umol, 20% yield, 99% purity) as an off-white solid.

1H NMR (400 MHz, DMSO-d6) δ=10.99 (s, 1H), 8.18-8.07 (m, 3H), 7.97-7.83 (m, 2H), 7.66 (s, 1H), 7.55 (s, 2H), 7.39-7.27 (m, 3H), 5.19 (s, 2H), 5.12 (dd, J=5.1, 13.3 Hz, 1H), 4.46-4.28 (m, 4H), 2.98-2.85 (m, 1H), 2.60 (br d, J=17.4 Hz, 1H), 2.40 (dq, J=4.4, 13.3 Hz, 1H), 2.05-1.94 (m, 1H). MS (ESI) m/z 503.3 [M+H]+.

### Example 5. Synthesis of Compound 5

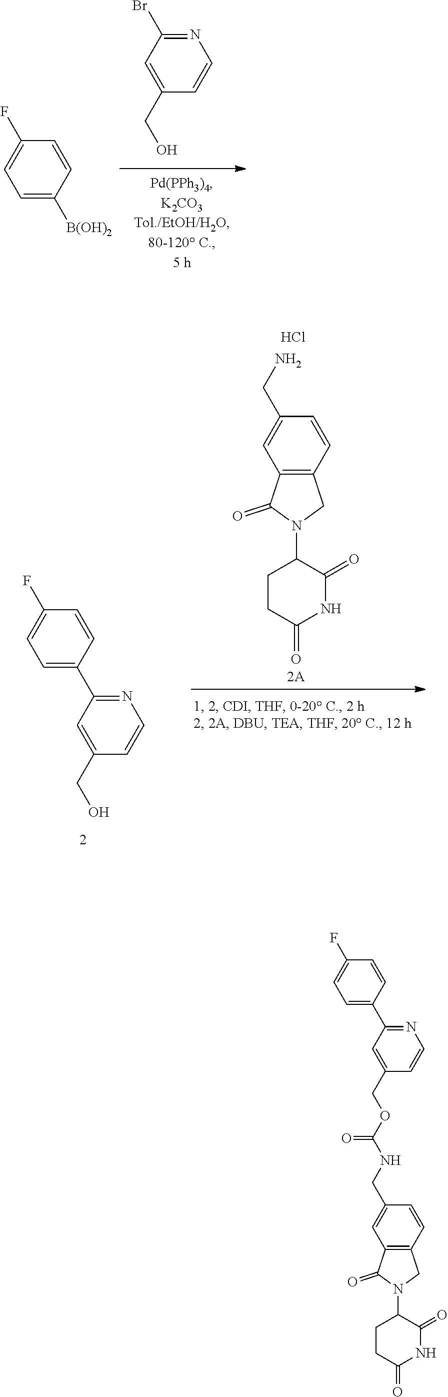 [SMILES: OB(O)c1ccc(F)cc1 ; OCc1ccnc(Br)c1 ; Cl ; OCc1ccnc(-c2ccc(F)cc2)c1 ; NCc1ccc2c(c1)C(=O)N(C1CCC(=O)NC1=O)C2 ; O=C1CCC(N2Cc3ccc(CNC(=O)OCc4ccnc(-c5ccc(F)cc5)c4)cc3C2=O)C(=O)N1]

### Step 1. Synthesis of (2-(4-fluorophenyl)pyridin-4-yl)methanol

To a solution of (2-bromopyridin-4-yl)methanol (1.00 g, 5.32 mmol, 1.00 eq) in toluene (12.0 mL), ethanol (1.00 mL) and water (2.00 mL) were added potassium carbonate (1.47 g, 10.6 mmol, 2.00 eq), tetrakis[triphenylphosphine]palladium(0) (307 mg, 266 umol, 0.0500 eq) and (4-fluorophenyl)boronic acid (1.04 g, 7.45 mmol, 1.40 eq). The reaction mixture was stirred at 80° C. for 2 h under nitrogen atmosphere. The reaction mixture was stirred at 120° C. for 3 h under nitrogen atmosphere. The reaction mixture was filtered and the filtrate was concentrated under reduced pressure to give a residue. The residue was purified by column chromatography (SiO₂, petroleum ether/ethyl acetate=1/0 to 3/1) to afford (2-(4-fluorophenyl)pyridin-4-yl)methanol (460 mg, 2.26 mmol, 43% yield) as yellow oil.

1H NMR (400 MHz, DMSO-d6) δ=8.58 (d, J=5.0 Hz, 1H), 8.16-8.08 (m, 2H), 7.86 (s, 1H), 7.35-7.27 (m, 3H), 5.48 (t, J=5.8 Hz, 1H), 4.61 (d, J=5.6 Hz, 2H).

### Step 2. Synthesis of (2-(4-fluorophenyl)pyridin-4-yl)methyl((2-(2,6-dioxopiperidin-3-yl)-3-oxoisoindolin-5-yl)methyl)carbamate

To a solution of (2-(4-fluorophenyl)pyridin-4-yl)methanol (50.0 mg, 246 umol, 1.00 eq) in tetrahydrofuran (0.500 mL) was added di(1H-imidazol-1-yl)methanone (79.8 mg, 492 umol, 2.00 eq). The mixture was stirred at 20° C. for 2 h. The resulting solution was added to a mixture of 3-(6-(aminomethyl)-1-oxoisoindolin-2-yl)piperidine-2,6-dione (76.2 mg, 246 umol, 1.00 eq, hydrochloride), 2,3,4,6,7,8,9,10-octahydropyrimido[1,2-a]azepine (37.5 mg, 246 umol, 37.1 uL, 1.00 eq) and triethylamine (24.9 mg, 246 umol, 34.3 uL, 1.00 eq) in tetrahydrofuran (0.500 mL). The reaction mixture was stirred at 20° C. for 12 h. The reaction mixture was concentrated under reduced pressure to give a residue. The residue was purified by Prep-HPLC(column: Unisil 3-100 C18 Ultra 150*50 mm*3 um; mobile phase: [water(formic acid)-acetonitrile]; B %: 20%-50%, 10 min) and lyophilized to give a crude product. The crude product was purified by Prep-HPLC(column: YMC Triart 30*150 mm*7 um; mobile phase: [water(hydrochloric acid)-acetonitrile]; B %: 17%-37%, 9 min) and lyophilized to afford (2-(4-fluorophenyl)pyridin-4-yl)methyl((2-(2,6-dioxopiperidin-3-yl)-3-oxoisoindolin-5-yl)methyl)carbamate (33.56 mg, 66.12 umol, 27% yield, 99% purity) as a white solid.

1H NMR (400 MHz, DMSO-d6) δ=10.99 (s, 1H), 8.72 (br dd, J=5.2, 14.9 Hz, 1H), 8.28-8.15 (m, 1H), 8.14-7.95 (m, 3H), 7.66 (s, 1H), 7.63-7.46 (m, 3H), 7.41 (td, J=8.9, 12.7 Hz, 2H), 5.26 (br d, J=14.2 Hz, 2H), 5.11 (dd, J=5.1, 13.3 Hz, 1H), 4.46-4.28 (m, 4H), 2.97-2.85 (m, 1H), 2.60 (br d, J=17.4 Hz, 1H), 2.40 (dq, J=4.3, 13.2 Hz, 1H), 2.05-1.94 (m, 1H). MS (ESI) m/z 503.3 [M+H]+.

### Example 6. Synthesis of Compound 6

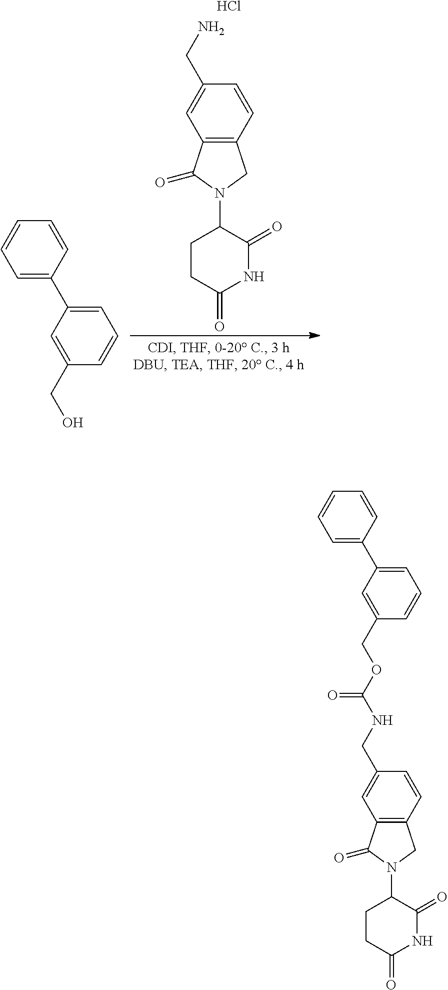 [SMILES: Cl ; OCc1cccc(-c2ccccc2)c1 ; NCc1ccc2c(c1)C(=O)N(C1CCC(=O)NC1=O)C2 ; O=C1CCC(N2Cc3ccc(CNC(=O)OCc4cccc(-c5ccccc5)c4)cc3C2=O)C(=O)N1]

### Step 1. [1,1′-biphenyl]-3-ylmethyl ((2-(2,6-dioxopiperidin-3-yl)-3-oxoisoindolin-5-yl)methyl)carbamate

To a solution of [1,1′-biphenyl]-3-ylmethanol (50.0 mg, 271 umol, 1.00 eq) in tetrahydrofuran (0.500 mL) was added di(1H-imidazol-1-yl)methanone (44.0 mg, 271 umol, 1.00 eq) in tetrahydrofuran (0.500 mL) at 0° C. The mixture was stirred at 20° C. for 3 h. Then the resulting solution was added to a suspension of 3-(6-(aminomethyl)-1-oxoisoindolin-2-yl)piperidine-2,6-dione (74.1 mg, 271 umol, 1.00 eq, hydrochloride), 3,4,5,7,8,9,10,10a-octahydropyrido[1,2-a][1,4]diazepine (41.3 mg, 271 umol, 40.9 uL, 1.00 eq), triethylamine (27.4 mg, 271 umol, 37.7 uL, 1.00 eq) in tetrahydrofuran (0.500 mL). The mixture was stirred at 20° C. for 4 h. The mixture was concentrated under reduced pressure to give a residue. The residue was purified by Prep-HPLC (column: Phenomenex Synergi C18 150*25 mm*10 um; mobile phase: [water(formic acid)-acetonitrile]; B %: 36%-66%, 10 min) and lyophilized to afford [1,1′-biphenyl]-3-ylmethyl ((2-(2,6-dioxopiperidin-3-yl)-3-oxoisoindolin-5-yl)methyl)carbamate (17.5 mg, 31.06 umol, 11% yield, 94% purity, formate) as an off-white solid.

1H NMR (400 MHz, DMSO-d6) δ=10.98 (br s, 1H), 8.47 (s, 1H), 7.98 (br t, J=6.1 Hz, 1H), 7.64 (s, 3H), 7.63-7.59 (m, 2H), 7.53 (s, 2H), 7.47 (t, J=7.5 Hz, 3H), 7.40-7.34 (m, 2H), 5.14-5.09 (m, 3H), 4.47-4.40 (m, 1H), 4.32 (s, 2H), 4.31-4.28 (m, 1H), 2.95-2.86 (m, 1H), 2.63-2.58 (m, 1H), 2.39 (m, 1H), 2.05-1.95 (m, 1H). MS (ESI) m/z 484.1 [M+H]+.

### Example 7. Synthesis of Compound 7

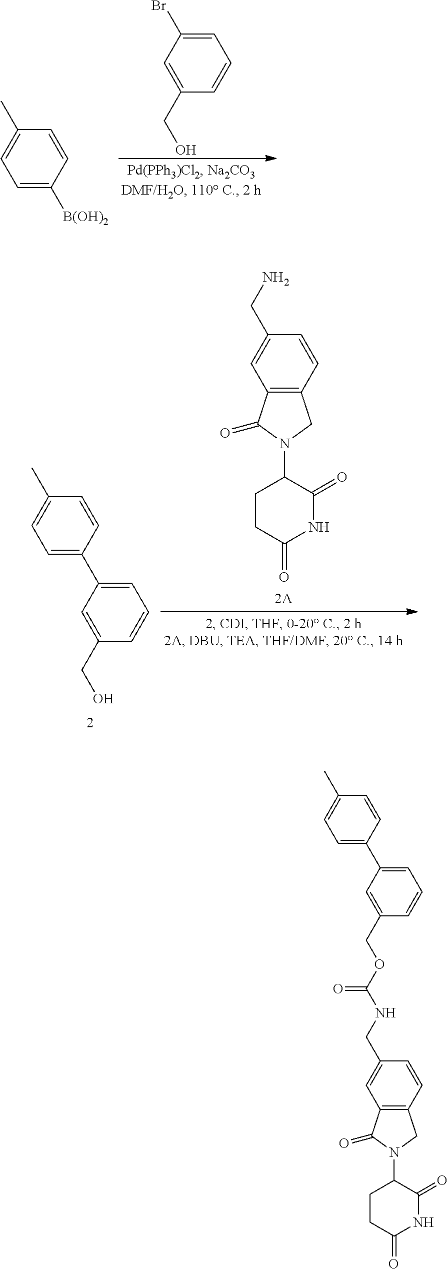

### Step 1. Synthesis of (4′-methyl-[1,1′-biphenyl]-3-yl)methanol

To a solution of (3-bromophenyl)methanol (1.00 g, 5.35 mmol, 641 uL, 1.00 eq), p-tolylboronic acid (872 mg, 6.42 mmol, 1.20 eq) in water (1.00 mL), dimethylformamide (8.00 mL) was added sodium carbonate (1.42 g, 13.3 mmol, 2.50 eq), bis(triphenylphosphine)palladium(II) chloride (750 mg, 1.07 mmol, 0.200 eq). The mixture was stirred at 110° C. for 2 h under nitrogen atmosphere. The reaction mixture was filtered. The filtrate was diluted with water (100 mL), extracted with ethyl acetate (3×50 mL). The combined organic layers were washed with brine (50 mL), dried over anhydrous sodium sulfate, filtered and concentrated under reduced pressure to give a residue. The residue was purified by column chromatography (petroleum ether/ethyl acetate=1/0 to 2/1) and concentrated under reduced pressure to afford (4′-methyl-[1,1′-biphenyl]-3-yl)methanol (600 mg, 3.03 mmol, 56% yield) as a white solid.

1H NMR (400 MHz, DMSO-d6) δ=7.58 (s, 1H), 7.54 (d, J=8.2 Hz, 2H), 7.49 (br d, J=7.7 Hz, 1H), 7.41-7.37 (m, 1H), 7.30-7.26 (m, 3H), 5.22 (t, J=5.7 Hz, 1H), 4.56 (d, J=5.7 Hz, 2H), 2.34 (s, 3H).

### Step 2. Synthesis of (4′-methyl-[1,1′-biphenyl]-3-yl)methyl ((2-(2,6-dioxopiperidin-3-yl)-3-oxoisoindolin-5-yl)methyl)carbamate

To a solution of (4′-methyl-[1,1′-biphenyl]-3-yl)methanol (50.0 mg, 252 umol, 1.00 eq) in tetrahydrofuran (0.500 mL) was added di(1H-imidazol-1-yl)methanone (81.7 mg, 504 umol, 2.00 eq) at 0° C. The mixture was stirred at 20° C. for 2 h. Then the resulting solution was added to a suspension of 3-(6-(aminomethyl)-1-oxoisoindolin-2-yl)piperidine-2,6-dione (78.1 mg, 252 umol, 1.00 eq, hydrochloride), triethylamine (25.5 mg, 252 umol, 35.1 uL, 1.00 eq), 2,3,4,6,7,8,9,10-octahydropyrimido[1,2-a]azepine (38.3 mg, 252 umol, 38.0 uL, 1.00 eq) in tetrahydrofuran (0.500 mL). The mixture was stirred at 20° C. for 4 h. After adding dimethylformamide (0.500 mL). The mixture was stirred at 20° C. for 10 h. The mixture was concentrated under reduced pressure to give a residue. The residue was filtered and the filtrate was purified by Prep-HPLC (Phenomenex Synergi C18 150*25 mm*10 um; mobile phase: [water(formic acid)-acetonitrile]; B %: 37%-67%, 10 min) and lyophilized to afford (4′-methyl-[1,1′-biphenyl]-3-yl)methyl ((2-(2,6-dioxopiperidin-3-yl)-3-oxoisoindolin-5-yl)methyl)carbamate (58.34 mg, 101.96 umol, 40% yield, 95% purity, formate) as an off-white solid.

1H NMR (400 MHz, DMSO-d6) δ=10.99 (br s, 1H), 8.48 (s, 1H), 7.98 (br t, J=6.2 Hz, 1H), 7.63 (br d, J=8.8 Hz, 2H), 7.58 (br d, J=7.8 Hz, 1H), 7.56-7.50 (m, 4H), 7.44 (t, J=7.6 Hz, 1H), 7.32 (br d, J=7.6 Hz, 1H), 7.27 (br d, J=7.8 Hz, 2H), 5.14-5.09 (m, 3H), 4.47-4.39 (m, 1H), 4.34-4.27 (m, 3H), 2.97-2.86 (m, 1H), 2.63-2.57 (m, 1H), 2.39 (m, 1H), 2.34 (s, 3H), 1.99 (m, 1H). MS (ESI) m/z 498.3 [M+H]+.

### Example 8. Synthesis of Compound 8

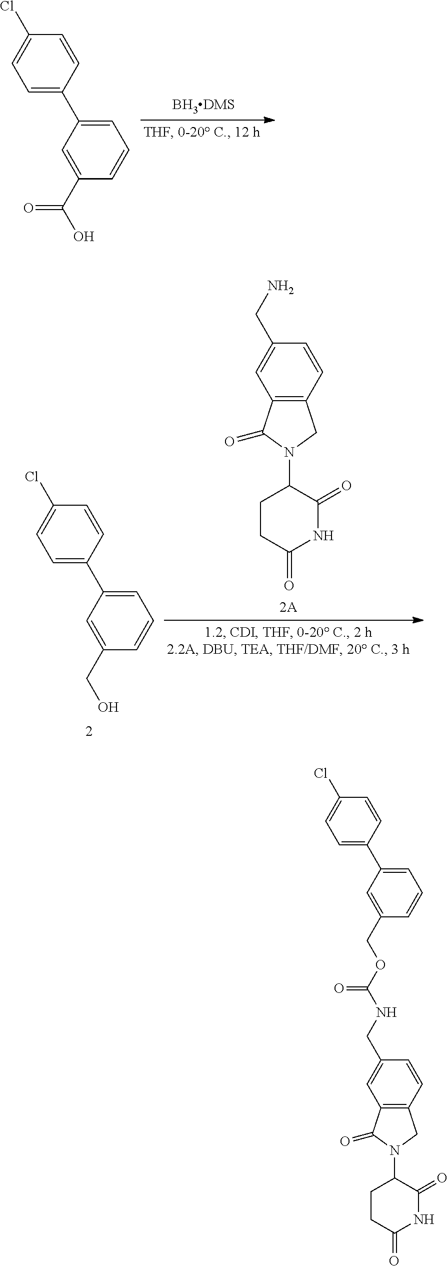 [SMILES: O=C(O)c1cccc(-c2ccc(Cl)cc2)c1 ; OCc1cccc(-c2ccc(Cl)cc2)c1 ; NCc1ccc2c(c1)C(=O)N(C1CCC(=O)NC1=O)C2 ; O=C1CCC(N2Cc3ccc(CNC(=O)OCc4cccc(-c5ccc(Cl)cc5)c4)cc3C2=O)C(=O)N1]

### Step 1. Synthesis of (4′-chloro-[1,1′-biphenyl]-3-yl)methanol

To a solution of 4′-chloro-[1,1′-biphenyl]-3-carboxylic acid (200 mg, 859 umol, 1.00 eq) in tetrahydrofuran (2.00 mL) was added borane dimethyl sulfide complex (10.0 M, 172 uL, 2.00 eq) at 0° C. slowly. The mixture was stirred at 20° C. for 12 h under nitrogen atmosphere. The mixture was quenched with methanol (10 mL) at 0° C. slowly and the mixture was concentrated under reduced pressure to give a residue. The residue was diluted with water (20 mL), extracted with ethyl acetate (2×20 mL). The combined organic layers were dried over anhydrous sodium sulfate, filtered and concentrated under reduced pressure to afford (4′-chloro-[1,1′-biphenyl]-3-yl)methanol (150 mg, 685 umol, 79% yield) as colorless oil.

1H NMR (400 MHz, DMSO-d6) δ=7.68 (br d, J=8.4 Hz, 2H), 7.60 (s, 1H), 7.55-7.49 (m, 3H), 7.42 (t, J=7.6 Hz, 1H), 7.33 (br d, J=7.6 Hz, 1H), 5.27 (t, J=5.7 Hz, 1H), 4.57 (d, J=5.7 Hz, 2H).

### Step 2. Synthesis of (4′-chloro-[1,1′-biphenyl]-3-yl)methyl ((2-(2,6-dioxopiperidin-3-yl)-3-oxoisoindolin-5-yl)methyl)carbamate

To a solution of (4′-chloro-[1,1′-biphenyl]-3-yl)methanol (50.0 mg, 228 umol, 1.00 eq) in tetrahydrofuran (0.500 mL) was added di(1H-imidazol-1-yl)methanone (74.1 mg, 457 umol, 2.00 eq) at 0° C. The mixture was stirred at 20° C. for 2 h. Then the resulting solution was added to a suspension of 3-(6-(aminomethyl)-1-oxoisoindolin-2-yl)piperidine-2,6-dione (70.8 mg, 228 umol, 1.00 eq, hydrochloride), triethylamine (23.1 mg, 228 umol, 31.8 uL, 1.00 eq), 3,4,5,7,8,9,10,10a-octahydropyrido[1,2-a][1,4]diazepine (34.8 mg, 228 umol, 34.4 uL, 1.00 eq) in tetrahydrofuran (0.500 mL). The mixture was stirred at 20° C. for 1 h. After adding dimethylformamide (0.500 mL), the mixture was stirred at 20° C. for 2 h. The mixture was concentrated under reduced pressure to remove tetrahydrofuran. The mixture was filtered and the filtrate was purified by Prep-HPLC (Phenomenex Synergi C18 150*25 mm*10 um; mobile phase: [water(formic acid)-acetonitrile]; B %: 38%-71%, 11 min) and lyophilized to afford (4′-chloro-[1,1′-biphenyl]-3-yl)methyl ((2-(2,6-dioxopiperidin-3-yl)-3-oxoisoindolin-5-yl)methyl)carbamate (52.09 mg, 99.56 umol, 43% yield, 99% purity) as a white solid.

1H NMR (400 MHz, DMSO-d6) δ=10.99 (s, 1H), 7.98 (br t, J=6.1 Hz, 1H), 7.68 (d, J=8.4 Hz, 2H), 7.65 (br d, J=6.1 Hz, 2H), 7.62 (br d, J=7.9 Hz, 1H), 7.55-7.50 (m, 4H), 7.50-7.45 (m, 1H), 7.38 (br d, J=7.7 Hz, 1H), 5.14-5.09 (m, 3H), 4.46-4.39 (m, 1H), 4.33-4.27 (m, 3H), 2.96-2.87 (m, 1H), 2.63-2.57 (m, 1H), 2.40 (br dd, J=4.5, 13.0 Hz, 1H), 2.04-1.96 (m, 1H). MS (ESI) m/z 518.3 [M+H]+.

### Example 9. Synthesis of Compound 9

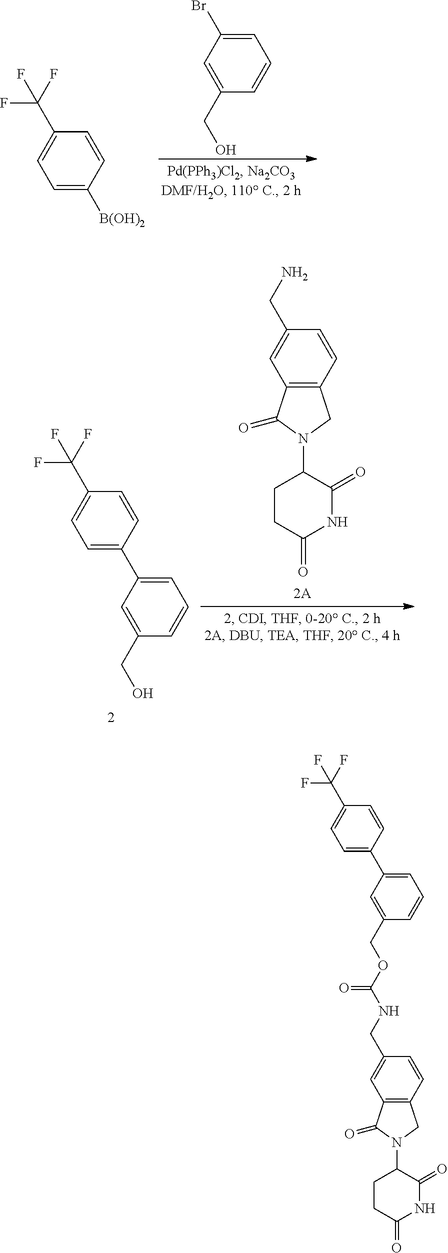 [SMILES: OB(O)c1ccc(C(F)(F)F)cc1 ; OCc1cccc(Br)c1 ; OCc1cccc(-c2ccc(C(F)(F)F)cc2)c1 ; NCc1ccc2c(c1)C(=O)N(C1CCC(=O)NC1=O)C2 ; O=C1CCC(N2Cc3ccc(CNC(=O)OCc4cccc(-c5ccc(C(F)(F)F)cc5)c4)cc3C2=O)C(=O)N1]

### Step 1. Synthesis of (4′-(trifluoromethyl)-[1,1′-biphenyl]-3-yl)methanol

To a solution of (3-bromophenyl)methanol (200 mg, 1.07 mmol, 128 uL, 1.00 eq), (4-(trifluoromethyl)phenyl)boronic acid (243 mg, 1.28 mmol, 1.20 eq) in water (0.500 mL), dimethylformamide (4.00 mL) was added sodium carbonate (283 mg, 2.67 mmol, 2.50 eq), bis(triphenylphosphine)palladium(II) chloride (150 mg, 213 umol, 0.200 eq). The mixture was stirred at 110° C. for 2 h under nitrogen atmosphere. The mixture was diluted with water (50 mL), extracted with ethyl acetate (3×30 mL). The combined organic layers were washed with brine (2×50 mL), dried over anhydrous sodium sulfate, filtered and concentrated under reduced pressure to give a residue. The residue was purified by column chromatography (SiO₂, petroleum ether/ethyl acetate=1/0 to 2/1) and concentrated under reduced pressure to afford (4′-(trifluoromethyl)-[1,1′-biphenyl]-3-yl)methanol (140 mg, 555 umol, 51% yield) as yellow oil.

1H NMR (400 MHz, DMSO-d6) δ=7.91-7.86 (m, 2H), 7.85-7.78 (m, 2H), 7.68 (s, 1H), 7.60 (br d, J=7.7 Hz, 1H), 7.47 (t, J=7.6 Hz, 1H), 7.41-7.37 (m, 1H), 5.28 (t, J=5.7 Hz, 1H), 4.59 (d, J=5.7 Hz, 2H).

### Step 2. Synthesis of (4′-(trifluoromethyl)-[1,1′-biphenyl]-3-yl)methyl((2-(2,6-dioxopiperidin-3-yl)-3-oxoisoindolin-5-yl)methyl)carbamate

To a solution of (4′-(trifluoromethyl)-[1,1′-biphenyl]-3-yl)methanol (40.0 mg, 158 umol, 1.00 eq) in tetrahydrofuran (0.500 mL) was added di(1H-imidazol-1-yl)methanone (51.4 mg, 317 umol, 2.00 eq) at 0° C. The mixture was stirred at 20° C. for 2 h. Then the resulting solution was added to a suspension of 3-(6-(aminomethyl)-1-oxoisoindolin-2-yl)piperidine-2,6-dione (49.0 mg, 158 umol, 1.00 eq, hydrochloride), 2,3,4,6,7,8,9,10-octahydropyrimido[1,2-a]azepine (24.1 mg, 158 umol, 23.9 uL, 1.00 eq), triethylamine (16.0 mg, 158 umol, 22.0 uL, 1.00 eq) in tetrahydrofuran (0.500 mL). The mixture was stirred at 20° C. for 4 h. The reaction mixture was concentrated under reduced pressure to give a residue. The residue was purified by Prep-HPLC (column: Phenomenex luna C18 150*25 mm*10 um; mobile phase: [water(formic acid)-acetonitrile]; B %: 43%-73%, 10 min) and lyophilized to afford (4′-(trifluoromethyl)-[1,1′-biphenyl]-3-yl)methyl((2-(2,6-dioxopiperidin-3-yl)-3-oxoisoindolin-5-yl)methyl)carbamate (11.72 mg, 21.04 umol, 13% yield, 99% purity, formate) as a white solid.

1H NMR (400 MHz, DMSO-d6) δ=10.98 (br s, 1H), 8.48 (s, 1H), 7.98 (br t, J=6.2 Hz, 1H), 7.91-7.86 (m, 2H), 7.82 (br d, J=8.4 Hz, 2H), 7.75-7.66 (m, 2H), 7.64 (s, 1H), 7.55-7.49 (m, 3H), 7.44 (br d, J=7.9 Hz, 1H), 5.16-5.08 (m, 3H), 4.46-4.39 (m, 1H), 4.35-4.27 (m, 3H), 2.89 (m, 1H), 2.63-2.58 (m, 1H), 2.42-2.37 (m, 1H), 2.03-1.96 (m, 1H). MS (ESI) m/z 552.1 [M+H]+.

### Example 10. Synthesis of Compound 10

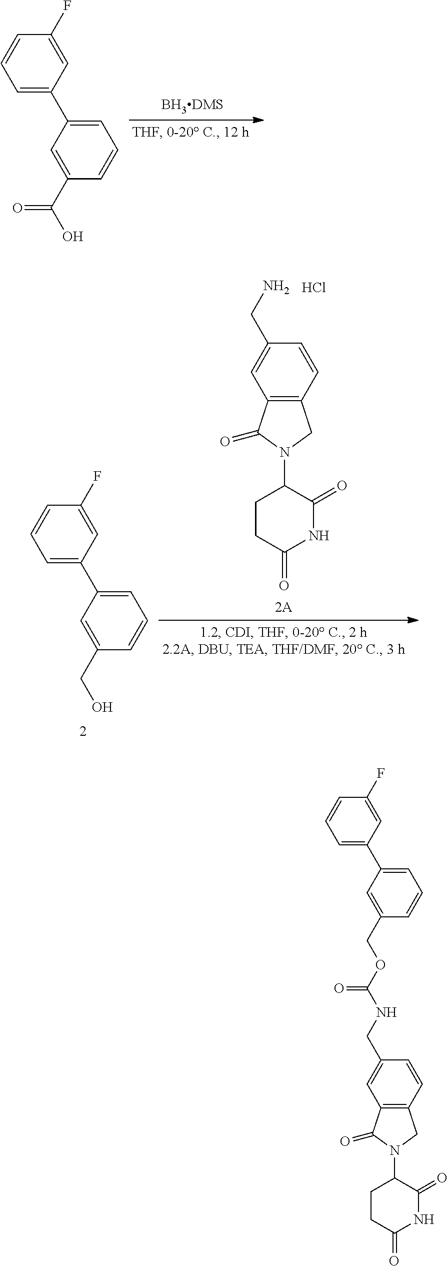 [SMILES: O=C(O)c1cccc(-c2cccc(F)c2)c1 ; OCc1cccc(-c2cccc(F)c2)c1 ; NCc1ccc2c(c1)C(=O)N(C1CCC(=O)NC1=O)C2 ; Cl ; O=C1CCC(N2Cc3ccc(CNC(=O)OCc4cccc(-c5cccc(F)c5)c4)cc3C2=O)C(=O)N1]

### Step 1. Synthesis of (3′-fluoro-[1,1′-biphenyl]-3-yl)methanol

To a solution of 3′-fluoro-[1,1′-biphenyl]-3-carboxylic acid (1.00 g, 4.63 mmol, 1.00 eq) in tetrahydrofuran (10.0 mL) was added borane dimethyl sulfide complex (10.0 M, 925 uL, 2.00 eq) at 0° C. slowly. The mixture was stirred at 20° C. for 12 h. The mixture was quenched with methanol (20 mL) at 0° C. slowly and the mixture was concentrated under reduced pressure to give a residue. The residue was diluted with water (50 mL), extracted with ethyl acetate (2×50 mL). The combined organic layers were dried over anhydrous sodium sulfate, filtered and concentrated under reduced pressure to give a residue. The residue was purified by reversed phase column chromatography (C18, 330 g; condition: water/acetonitrile=1/0 to 0/1, 0.1% ammonium hydroxide) and lyophilized to afford (3′-fluoro-[1,1′-biphenyl]-3-yl)methanol (100 mg, 346 umol, 7% yield, 70% purity) as a white solid.

1H NMR (400 MHz, DMSO-d6) δ=7.67 (d, J=8.2 Hz, 2H), 7.52-7.48 (m, 3H), 7.43-7.39 (m, 2H), 7.20-7.15 (m, 1H), 5.28-5.23 (m, 1H), 4.54 (d, J=5.7 Hz, 2H).

### Step 2. Synthesis of (3′-fluoro-[1,1′-biphenyl]-3-yl)methyl ((2-(2,6-dioxopiperidin-3-yl)-3-oxoisoindolin-5-yl)methyl)carbamate

To a solution of (3′-fluoro-[1,1′-biphenyl]-3-yl)methanol (20.0 mg, 69.2 umol, 70% purity, 1.00 eq) in tetrahydrofuran (0.500 mL) was added di(1H-imidazol-1-yl)methanone (22.4 mg, 138 umol, 2.00 eq) at 0° C. The mixture was stirred at 20° C. for 2 h. Then the resulting solution was added to a suspension of 3-(6-(aminomethyl)-1-oxoisoindolin-2-yl)piperidine-2,6-dione (21.4 mg, 69.2 umol, 1.00 eq, hydrochloride), triethylamine (7.01 mg, 69.2 umol, 9.64 uL, 1.00 eq) and 3,4,5,7,8,9,10,10a-octahydropyrido[1,2-a][1,4]diazepine (10.5 mg, 69.2 umol, 10.4 uL, 1.00 eq) in tetrahydrofuran (0.500 mL) which was stirred at 20° C. for 1 h. After adding dimethylformamide (0.500 mL), the mixture was stirred at 20° C. for 2 h. The mixture was concentrated under reduced pressure to remove tetrahydrofuran. The mixture was filtered and the filtrate was purified by Prep-HPLC (Phenomenex Synergi C18 150*25 mm*10 um; mobile phase: [water (formic acid)-acetonitrile]; B %: 37%-67%, 10 min) and lyophilized to afford (3′-fluoro-[1,1′-biphenyl]-3-yl)methyl ((2-(2,6-dioxopiperidin-3-yl)-3-oxoisoindolin-5-yl)methyl)carbamate (17.07 mg, 30.55 umol, 44% yield, 98% purity, formate) as an off-white solid.

1H NMR (400 MHz, DMSO-d6) δ=10.99 (br s, 1H), 8.49 (s, 1H), 7.99 (br t, J=6.1 Hz, 1H), 7.73-7.64 (m, 3H), 7.57-7.49 (m, 5H), 7.48-7.38 (m, 2H), 7.24-7.17 (m, 1H), 5.14-5.08 (m, 3H), 4.47-4.40 (m, 1H), 4.33-4.28 (m, 3H), 2.96-2.86 (m, 1H), 2.60 (br dd, J=2.1, 15.2 Hz, 1H), 2.43-2.35 (m, 1H), 2.04-1.95 (m, 1H). MS (ESI) m/z 502.3 [M+H]+.

### Example 11. Synthesis of Compound 11

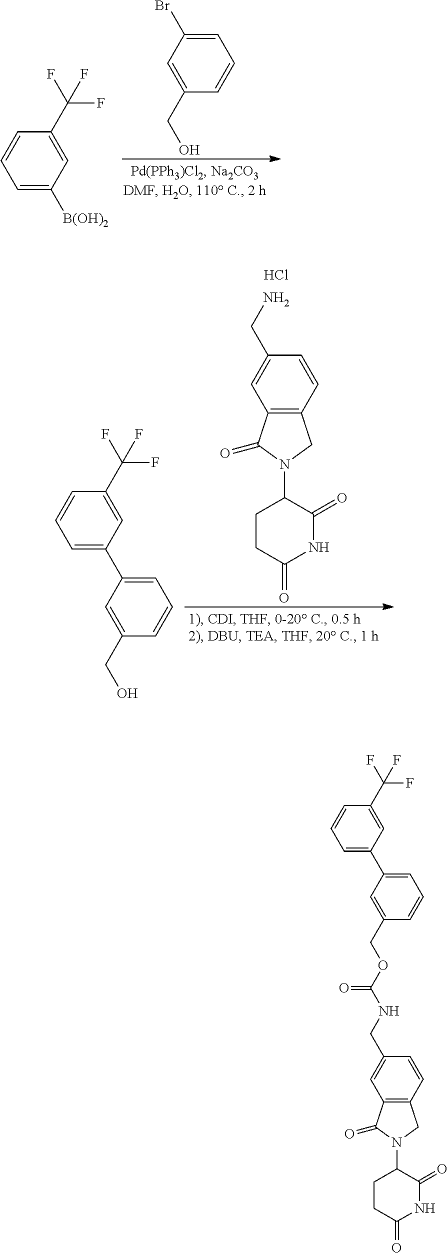 [SMILES: OB(O)c1cccc(C(F)(F)F)c1 ; OCc1cccc(Br)c1 ; Cl ; OCc1cccc(-c2cccc(C(F)(F)F)c2)c1 ; NCc1ccc2c(c1)C(=O)N(C1CCC(=O)NC1=O)C2 ; O=C1CCC(N2Cc3ccc(CNC(=O)OCc4cccc(-c5cccc(C(F)(F)F)c5)c4)cc3C2=O)C(=O)N1]

### Step 1. Synthesis of (3′-(trifluoromethyl)-[1,1′-biphenyl]-3-yl)methanol

To a solution of (3-bromophenyl)methanol (1.00 g, 5.35 mmol, 641 uL, 1.00 eq) in dimethyformamide (20.0 mL) and water (2.50 mL) were added (3-(trifluoromethyl)phenyl)boronic acid (1.22 g, 6.42 mmol, 1.20 eq), bis(triphenylphosphine)palladium(II) (751 mg, 1.07 mmol, 0.200 eq) and sodium carbonate (1.42 g, 13.4 mmol, 2.50 eq). The reaction mixture was stirred at 110° C. for 2 h under nitrogen atmosphere. The reaction mixture was quenched with water (50 mL) and extracted with ethyl acetate (3×100 mL). The combined organic layers were washed with brine (100 mL), dried over anhydrous sodium sulfate, filtered and concentrated under reduced pressure to give a residue. The residue was purified by chromatography on silica gel (petroleum ether/ethyl acetate=1/0 to 3/1) to afford (3′-(trifluoromethyl)-[1,1′-biphenyl]-3-yl)methanol (1.10 g, 4.36 mmol, 82% yield) as yellow oil.

1H NMR (400 MHz, CDCl3) δ=7.76 (s, 1H), 7.69 (d, J=7.6 Hz, 1H), 7.57-7.51 (m, 2H), 7.47 (dd, J=7.6, 15.2 Hz, 2H), 7.39 (t, J=7.5 Hz, 1H), 7.34-7.30 (m, 1H), 4.71 (s, 2H).

### Step 2. Synthesis of (3′-(trifluoromethyl)-[1,1′-biphenyl]-3-yl)methyl ((2-(2,6-dioxopiperidin-3-yl)-3-oxoisoindolin-5-yl)methyl)carbamate

To a solution of (3′-(trifluoromethyl)-[1,1′-biphenyl]-3-yl)methanol (100 mg, 396 umol, 1.00 eq) in tetrahydrofuran (1.00 mL) was added 1,1′-carbonyldiimidazole (129 mg, 793 umol, 2.00 eq) at 0° C. The reaction mixture was stirred at 20° C. for 0.5 h. Then the reaction mixture was added into a solution of 3-(6-(aminomethyl)-1-oxoisoindolin-2-yl)piperidine-2,6-dione (123 mg, 396 umol, 1.00 eq, hydrochloric acid), 2,3,4,6,7,8,9,10-octahydropyrimido[1,2-a]azepine (60.4 mg, 396 umol, 59.8 uL, 1.00 eq) and triethylamine (40.1 mg, 396 umol, 55.2 uL, 1.00 eq) in tetrahydrofuran (0.500 mL). The reaction mixture was stirred at 20° C. for 1 h. The reaction mixture was filtrated. The filtrate was purified by Prep-HPLC (column: Phenomenex Synergi C18 150*25 mm*10 um; mobile phase: [water(formic acid)-acetonitrile]; B %: 50%-70%, 10 min) and lyophilized to afford (3′-(trifluoromethyl)-[1,1′-biphenyl]-3-yl)methyl ((2-(2,6-dioxopiperidin-3-yl)-3-oxoisoindolin-5-yl)methyl)carbamate (32.61 mg, 54.03 umol, 14% yield, 99% purity, formate) as a white solid.

1H NMR (400 MHz, DMSO-d6) δ=11.0 (br s, 1H), 8.46 (br d, J=1.8 Hz, 1H), 8.04-7.89 (m, 3H), 7.81-7.67 (m, 4H), 7.64 (s, 1H), 7.58-7.47 (m, 3H), 7.47-7.39 (m, 1H), 5.28-5.03 (m, 3H), 4.53-4.19 (m, 4H), 2.97-2.85 (m, 1H), 2.60 (br dd, J=1.4, 17.5 Hz, 1H), 2.45-2.37 (m, 1H), 2.04-1.92 (m, 1H). MS (ESI) m/z 552.3 [M+H]+.

### Example 12. Synthesis of Compound 12

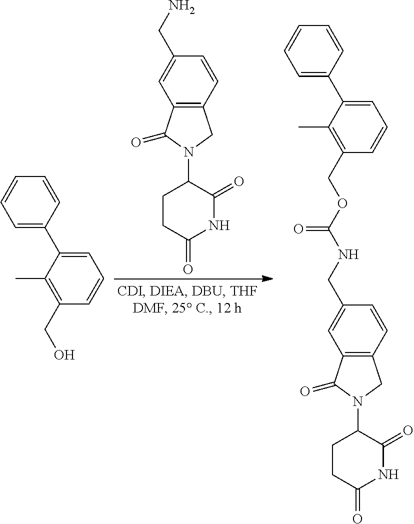 [SMILES: Cc1c(CO)cccc1-c1ccccc1 ; NCc1ccc2c(c1)C(=O)N(C1CCC(=O)NC1=O)C2 ; Cc1c(COC(=O)NCc2ccc3c(c2)C(=O)N(C2CCC(=O)NC2=O)C3)cccc1-c1ccccc1]

### Step 1. Synthesis of (2-methyl-[1,1′-biphenyl]-3-yl)methyl ((2-(2,6-dioxopiperidin-3-yl)-3-oxoisoindolin-5-yl)methyl)carbamate

To a solution of (2-methyl-[1,1′-biphenyl]-3-yl)methanol (200 mg, 1.01 mmol, 1.00 eq) in tetrahydrofuran (1.00 mL) was added 1,1′-carbonyldiimidazole (327 mg, 2.02 mmol, 2.00 eq). The mixture was stirred at 25° C. for 2 h. The resulting solution was added to a mixture of 3-(6-(aminomethyl)-1-oxoisoindolin-2-yl)piperidine-2,6-dione (276 mg, 890 umol, 8.82e-1 eq, hydrochloride), 2,3,4,6,7,8,9,10-octahydropyrimido[1,2-a]azepine (154 mg, 1.01 mmol, 152 uL, 1.00 eq) and triethylamine (102 mg, 1.01 mmol, 140 uL, 1.00 eq) in tetrahydrofuran (1.00 mL) and dimethylformamide (1.00 mL). The reaction mixture was stirred at 25° C. for 10 h. The reaction mixture was filtered. The filtrate was purified by Prep-HPLC (formic acid; column: Unisil 3-100 C18 Ultra 150×50 mm×3 um; mobile phase: [water(formic acid)-acetonitrile]; B %: 37%-67%, 15 min) to give a residue. The residue was purified by Prep-HPLC (neutral condition; column: Waters Xbridge 150×25 mm×5 um; mobile phase: [water (ammonium bicarbonate)-acetonitrile]; B %: 30%-60%, 10 min) to afford (2-methyl-[1,1′-biphenyl]-3-yl)methyl ((2-(2,6-dioxopiperidin-3-yl)-3-oxoisoindolin-5-yl)methyl)carbamate (111.8 mg, 201.57 umol, 20% yield, 98% purity, formate) as a white solid.

1H NMR (400 MHz, DMSO-d6) δ=10.99 (br s, 1H), 8.43 (s, 1H), 8.00 (br t, J=6.2 Hz, 1H), 7.65 (s, 1H), 7.55 (br d, J=5.4 Hz, 2H), 7.48-7.44 (m, 2H), 7.40-7.36 (m, 2H), 7.30-7.25 (m, 3H), 7.17 (br d, J=7.6 Hz, 1H), 5.14 (s, 2H), 5.11 (d, J=5.0 Hz, 1H), 4.48-4.42 (m, 1H), 4.33 (s, 2H), 4.32 (br s, 1H), 2.96-2.88 (m, 1H), 2.63-2.58 (m, 1H), 2.40 (dd, J=4.2, 13.2 Hz, 1H), 2.16 (s, 3H), 2.02-1.99 (m, 1H). MS (ESI) m/z 498.3 [M+H]+.

### Example 13. Synthesis of Compound 13

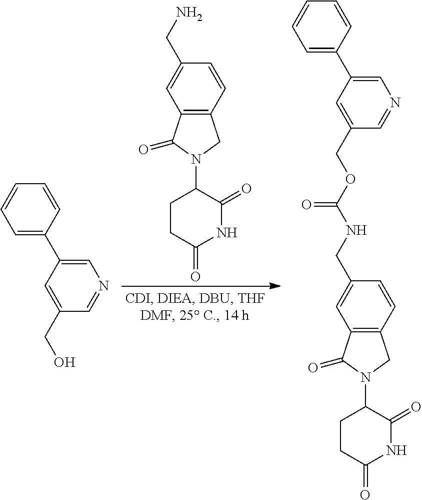 [SMILES: OCc1cncc(-c2ccccc2)c1 ; NCc1ccc2c(c1)C(=O)N(C1CCC(=O)NC1=O)C2 ; O=C1CCC(N2Cc3ccc(CNC(=O)OCc4cncc(-c5ccccc5)c4)cc3C2=O)C(=O)N1]

### Step 1. Synthesis of (5-phenylpyridin-3-yl)methyl ((2-(2,6-dioxopiperidin-3-yl)-3-oxoisoindolin-5-yl)methyl)carbamate

To a mixture of (5-phenylpyridin-3-yl) methanol (100 mg, 540 umol, 1.00 eq) in tetrahydrofuran (0.500 mL) was added di(1H-imidazol-1-yl)methanone (175 mg, 1.08 mmol, 2.00 eq). The mixture was stirred at 25° C. for 2 h. The resulting solution was added to a mixture of 3-(6-(aminomethyl)-1-oxoisoindolin-2-yl)piperidine-2,6-dione (167 mg, 540 umol, 1.00 eq, hydrochloride), triethylamine (54.6 mg, 540 umol, 75.2 uL, 1.00 eq) and 2,3,4,6,7,8,9,10-octahydropyrimido[1,2-a]azepine (82.2 mg, 540 umol, 81.4 uL, 1.00 eq) in tetrahydrofuran (0.500 mL) and dimethylformamide (0.500 mL). The reaction mixture was stirred at 25° C. for 12 h. The reaction mixture was filtered. The filtrate was purified by Prep-HPLC (formic acid; column: Unisil 3-100 C18 Ultra 150×50 mm×3 um; mobile phase: [water (formic acid)-acetonitrile]; B %: 15%-45%, 10 min) to afford (5-phenylpyridin-3-yl)methyl ((2-(2,6-dioxopiperidin-3-yl)-3-oxoisoindolin-5-yl)methyl)carbamate (90.8 mg, 168 umol, 31% yield, 98% purity, formate) as a white solid.

1H NMR (400 MHz, DMSO-d6) δ=11.00 (br s, 1H), 8.85 (d, J=2.0 Hz, 1H), 8.59 (d, J=1.2 Hz, 1H), 8.43 (s, 1H), 8.10-8.00 (m, 2H), 7.72 (d, J=7.6 Hz, 2H), 7.65 (s, 1H), 7.55-7.50 (m, 4H), 7.47-7.42 (m, 1H), 5.18 (s, 2H), 5.12 (dd, J=5.0, 13.3 Hz, 1H), 4.47-4.41 (m, 1H), 4.32 (br d, J=5.2 Hz, 3H), 2.97-2.88 (m, 1H), 2.64-2.58 (m, 1H), 2.40 (br dd, J=4.0, 12.8 Hz, 1H), 2.05-1.98 (m, 1H). MS (ESI) m/z 485.2 [M+H]+.

### Example 14. Synthesis of Compound 14

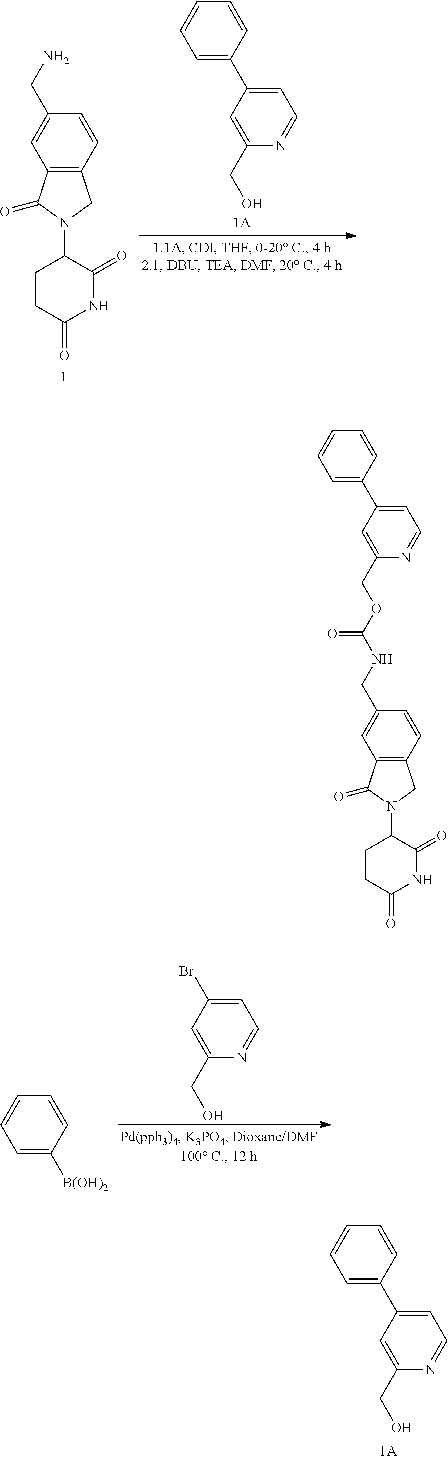 [SMILES: NCc1ccc2c(c1)C(=O)N(C1CCC(=O)NC1=O)C2 ; OCc1cc(-c2ccccc2)ccn1 ; O=C1CCC(N2Cc3ccc(CNC(=O)OCc4cc(-c5ccccc5)ccn4)cc3C2=O)C(=O)N1 ; OB(O)c1ccccc1 ; OCc1cc(Br)ccn1 ; OCc1cc(-c2ccccc2)ccn1]

### Step 1. Synthesis of (4-phenylpyridin-2-yl)methanol

To a solution of phenylboronic acid (486 mg, 3.99 mmol, 1.50 eq), (4-bromopyridin-2-yl)methanol (500 mg, 2.66 mmol, 1.00 eq) in dioxane (4.00 mL), dimethylformamide (1.00 mL) was added potassium phosphate (1.69 g, 7.98 mmol, 3.00 eq), tetrakis[triphenylphosphine]palladium(0) (307 mg, 265 umol, 0.100 eq). The mixture was stirred at 100° C. for 12 h under nitrogen atmosphere. The mixture was filtered. The filtrate was diluted with water (20 mL), extracted with ethyl acetate (3×20 mL). The combined organic layers were washed with brine (50 mL), dried over anhydrous sodium sulfate, filtered and concentrated under reduced pressure to give a residue. The residue was purified by column chromatography (petroleum ether/ethyl acetate=1/0 to 0/1) to give a crude product. The crude product was re-purified reversed phase column chromatography (C18, 80 g; condition: water/acetonitrile=1/0 to 0/1, 0.1% ammonium hydroxide) and lyophilized to afford (4-phenylpyridin-2-yl)methanol (100 mg, 539 umol, 20% yield) as a yellow solid.

1H NMR (400 MHz, DMSO-d6) δ=8.54 (d, J=5.0 Hz, 1H), 7.80-7.76 (m, 2H), 7.74 (d, J=0.9 Hz, 1H), 7.58-7.51 (m, 3H), 7.50-7.45 (m, 1H), 5.48 (t, J=5.9 Hz, 1H), 4.62 (d, J=5.9 Hz, 2H).

### Step 2. Synthesis of (4-phenylpyridin-2-yl)methyl ((2-(2,6-dioxopiperidin-3-yl)-3-oxoisoindolin-5-yl)methyl)carbamate

To a solution of (4-phenylpyridin-2-yl)methanol (40.0 mg, 215 umol, 1.00 eq) in tetrahydrofuran (0.500 mL) was added di(1H-imidazol-1-yl)methanone (70.0 mg, 431 umol, 2.00 eq). The mixture was stirred at 20° C. for 4 h. Then the resulting solution was added to a suspension of 3-(6-(aminomethyl)-1-oxoisoindolin-2-yl)piperidine-2,6-dione (66.8 mg, 215 umol, 1.00 eq, hydrochloride), triethylamine (21.8 mg, 215 umol, 30.0 uL, 1.00 eq), 3,4,5,7,8,9,10,10a-octahydropyrido[1,2-a] [1,4]diazepine (32.8 mg, 215 umol, 32.5 uL, 1.00 eq) in dimethylformamide (0.500 mL). The mixture was stirred at 20° C. for 4 h. The mixture was concentrated under reduced pressure to remove tetrahydrofuran. The mixture was filtered and the filtrate was purified by Prep-HPLC (Phenomenex Synergi C18 150*25 mm*10 um; mobile phase: [water(formic acid)-acetonitrile]; B %: 16%-46%, 10 min) and lyophilized to afford (4-phenylpyridin-2-yl)methyl((2-(2,6-dioxopiperidin-3-yl)-3-oxoisoindolin-5-yl)methyl)carbamate (29.34 mg, 54.2 umol, 25% yield, 98% purity, formic acid) as a white solid.

1H NMR (400 MHz, DMSO-d6) δ=10.99 (br s, 1H), 8.62 (d, J=5.1 Hz, 1H), 8.31 (s, 1H), 8.10 (br t, J=6.2 Hz, 1H), 7.75 (br d, J=6.8 Hz, 2H), 7.69-7.61 (m, 3H), 7.56-7.47 (m, 5H), 5.18 (s, 2H), 5.12 (m, 1H), 4.46-4.40 (m, 1H), 4.35-4.27 (m, 3H), 2.95-2.86 (m, 1H), 2.63-2.59 (m, 1H), 2.39 (m, 1H), 2.04-1.95 (m, 1H). MS (ESI) m/z 485.2 [M+H]+.

### Example 15. Synthesis of Compound 15

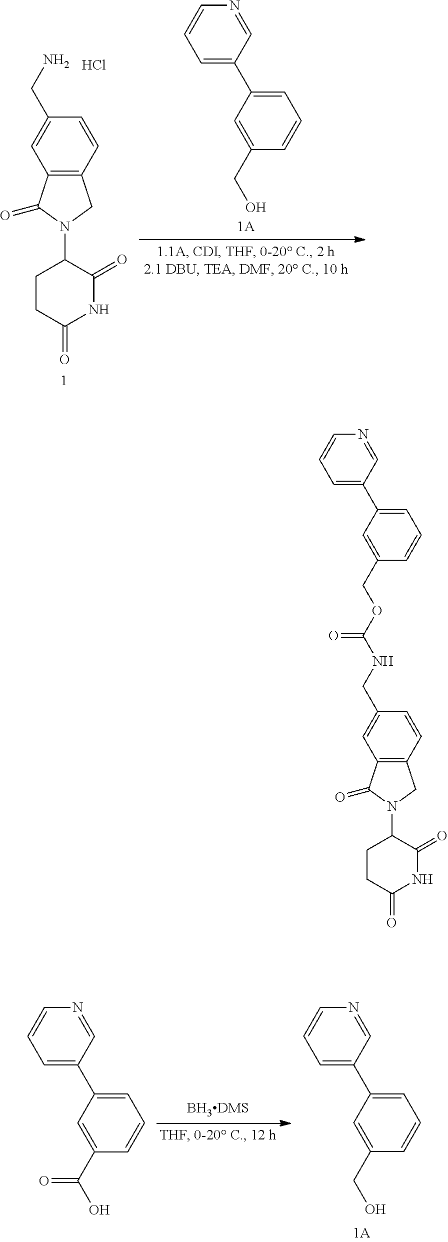 [SMILES: NCc1ccc2c(c1)C(=O)N(C1CCC(=O)NC1=O)C2 ; Cl ; OCc1cccc(-c2cccnc2)c1 ; O=C1CCC(N2Cc3ccc(CNC(=O)OCc4cccc(-c5cccnc5)c4)cc3C2=O)C(=O)N1 ; O=C(O)c1cccc(-c2cccnc2)c1 ; OCc1cccc(-c2cccnc2)c1]

### Step 1. Synthesis of (3-(pyridin-3-yl)phenyl)methanol

To a solution of 3-(pyridin-3-yl)benzoic acid (300 mg, 1.51 mmol, 1.00 eq) in tetrahydrofuran (2.00 mL) was added borane dimethyl sulfide complex (10 M, 301 uL, 2.00 eq) at 0° C. slowly. The mixture was stirred at 20° C. for 12 h under nitrogen atmosphere. The mixture was quenched with methanol (10 mL) at 0° C. slowly and the mixture was concentrated under reduced pressure to give a residue. The residue was diluted with water (20 mL) and extracted with ethyl acetate (2×20 mL). The combined organic layers were dried over anhydrous sodium sulfate, filtered and concentrated under reduced pressure to afford (3-(pyridin-3-yl)phenyl)methanol (150 mg, 809 umol, 53% yield) as a white solid.

1H NMR (400 MHz, DMSO-d6) δ=8.79 (s, 1H), 8.58 (d, J=5.5 Hz, 1H), 8.46 (br d, J=8.1 Hz, 1H), 7.81 (dd, J=5.7, 7.9 Hz, 1H), 7.72 (s, 1H), 7.66 (br d, J=7.5 Hz, 1H), 7.53-7.48 (m, 1H), 7.47-7.43 (m, 1H), 4.60 (s, 2H).

### Step 2. Synthesis of 3-(pyridin-3-yl)benzyl ((2-(2,6-dioxopiperidin-3-yl)-3-oxoisoindolin-5-yl)methyl)carbamate

To a solution of (3-(pyridin-3-yl)phenyl)methanol (40.0 mg, 216 umol, 1.00 eq) in tetrahydrofuran (0.500 mL) was added di(1H-imidazol-1-yl)methanone (70.0 mg, 431 umol, 2.00 eq). The mixture was stirred at 20° C. for 2 h. Then the resulting solution was added to a suspension of 3-(6-(aminomethyl)-1-oxoisoindolin-2-yl)piperidine-2,6-dione (66.8 mg, 216 umol, 1.00 eq, hydrochloride), triethylamine (21.8 mg, 216 umol, 30.0 uL, 1.00 eq), 3,4,5,7,8,9,10,10a-octahydropyrido[1,2-a][1,4]diazepine (32.8 mg, 216 umol, 32.5 uL, 1.00 eq) in dimethylformamide (0.500 mL). The mixture was stirred at 20° C. for 10 h. The mixture was concentrated under reduced pressure to remove tetrahydrofuran. The mixture was filtered and the filtrate was purified by Prep-HPLC (Phenomenex Synergi C18 150*25 mm*10 um; mobile phase: [water(formic acid)-acetonitrile]; B %: 3%-35%, 11 min) and lyophilized to afford 3-(pyridin-3-yl)benzyl ((2-(2,6-dioxopiperidin-3-yl)-3-oxoisoindolin-5-yl)methyl)carbamate (29.37 mg, 54.8 umol, 25% yield, 99% purity, formate) as a white solid.

1H NMR (400 MHz, DMSO-d6) δ=10.98 (br s, 1H), 8.88 (d, J=1.8 Hz, 1H), 8.58 (dd, J=1.5, 4.8 Hz, 1H), 8.27 (br s, 1H), 8.05 (br d, J=7.9 Hz, 1H), 7.98 (br t, J=6.2 Hz, 1H), 7.71 (s, 1H), 7.68 (br d, J=7.8 Hz, 1H), 7.64 (s, 1H), 7.53 (s, 2H), 7.51-7.47 (m, 2H), 7.42 (br d, J=7.6 Hz, 1H), 5.15-5.09 (m, 3H), 4.46-4.40 (m, 1H), 4.33-4.28 (m, 3H), 2.96-2.87 (m, 1H), 2.63-2.57 (m, 1H), 2.40 (m, 1H), 2.03-1.97 (m, 1H). MS (ESI) m/z 485.2 [M+H]+.

### Example 16. Synthesis of Compound 16

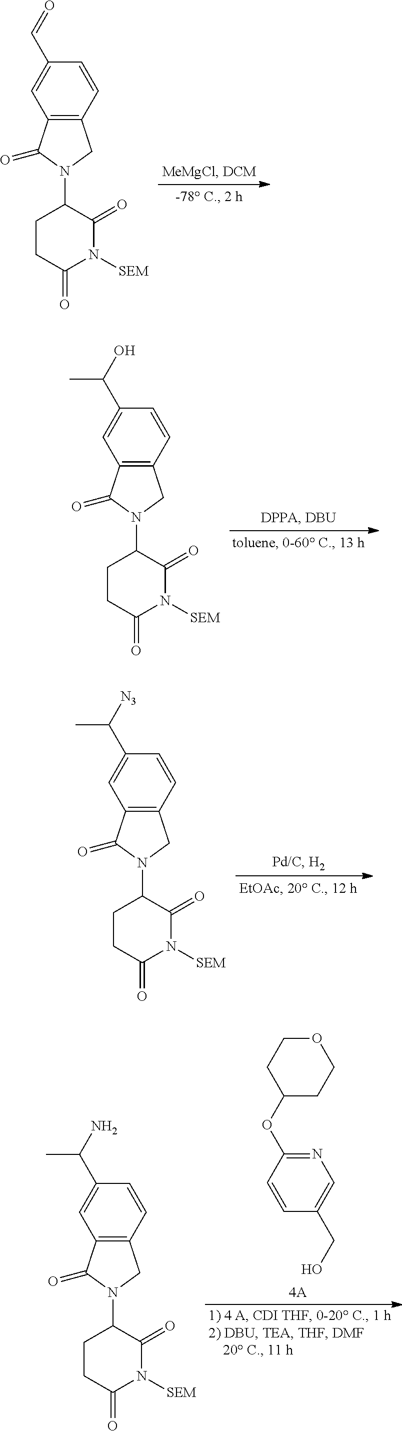

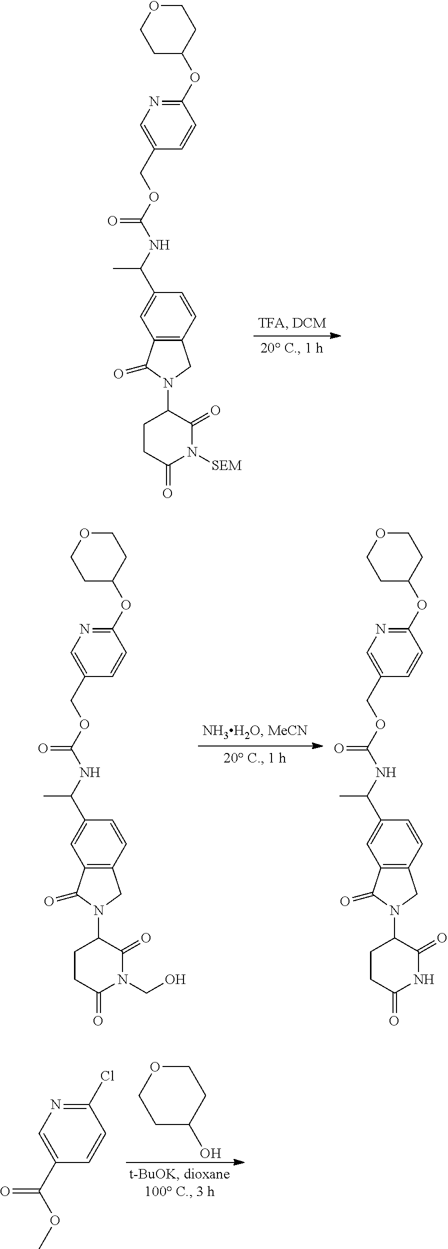 [SMILES: CC(NC(=O)OCc1ccc(OC2CCOCC2)nc1)c1ccc2c(c1)C(=O)N(C1CCC(=O)N(COCC[Si](C)(C)C)C1=O)C2 ; CC(NC(=O)OCc1ccc(OC2CCOCC2)nc1)c1ccc2c(c1)C(=O)N(C1CCC(=O)N(CO)C1=O)C2 ; CC(NC(=O)OCc1ccc(OC2CCOCC2)nc1)c1ccc2c(c1)C(=O)N(C1CCC(=O)NC1=O)C2 ; COC(=O)c1ccc(Cl)nc1 ; OC1CCOCC1]

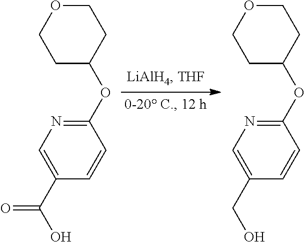 [SMILES: O=C(O)c1ccc(OC2CCOCC2)nc1 ; OCc1ccc(OC2CCOCC2)nc1]

### Step 1. Synthesis of 6-((tetrahydro-2H-pyran-4-yl)oxy)nicotinic acid

To a solution of methyl 6-chloronicotinate (10.0 g, 58.3 mmol, 1.00 eq) in dioxane (50.0 mL) was added tetrahydro-2H-pyran-4-ol (17.9 g, 174.8 mmol, 17.5 mL, 3.00 eq) and potassium tert-butoxide (19.6 g, 175 mmol, 3.00 eq). The reaction mixture was stirred at 100° C. for 3 h. The mixture was quenched with water (100 mL) and extracted with ethyl acetate (2×300 mL). The aqueous phase was adjusted pH=4 with 1 N hydrochloric acid and extracted with ethyl acetate (3×300 mL). The combined organic layers were washed with brine (100 mL), dried over sodium sulfate, filtered and concentrated under reduced pressure to give a crude product. The crude product was purified by reverse phase chromatography (C18, 80 g; condition: water/acetonitrile=100:0 to 0:100, 0.1% formic acid) and lyophilized to afford 6-((tetrahydro-2H-pyran-4-yl)oxy)nicotinic acid (3.70 g, 16.6 mmol, 28% yield) as a white solid.

1H NMR (400 MHz, CDCl3) δ=8.89 (d, J=2.2 Hz, 1H), 8.21 (dd, J=2.4, 8.7 Hz, 1H), 6.78 (d, J=8.7 Hz, 1H), 5.37 (tt, J=4.1, 8.5 Hz, 1H), 4.02 (td, J=4.6, 11.6 Hz, 2H), 3.65 (ddd, J=2.9, 9.1, 11.8 Hz, 2H), 2.17-1.96 (m, 2H), 1.93-1.65 (m, 2H).

### Step 2. (6-((tetrahydro-2H-pyran-4-yl)oxy)pyridin-3-yl)methanol

To a solution of 6-((tetrahydro-2H-pyran-4-yl)oxy)nicotinic acid (2.00 g, 8.96 mmol, 1.00 eq) in tetrahydrofuran (20.0 mL) was added lithium aluminum hydride (1.02 g, 26.9 mmol, 3.00 eq) at 0° C. The reaction mixture was stirred at 20° C. for 12 h. The mixture was quenched with sodium sulfafe decahydrate (10.0 g), filtered and concentrated under reduced pressure to give a residue. The residue was purified by column chromatography (SiO2, petroleum ether/ethyl acetate=1/0 to 1/1) to afford (6-((tetrahydro-2H-pyran-4-yl)oxy) pyridin-3-yl)methanol (200 mg, 955.84 umol, 10.67% yield) as a white solid.

1H NMR (400 MHz, CDCl3) δ=8.08 (d, J=2.0 Hz, 1H), 7.61 (dd, J=2.3, 8.4 Hz, 1H), 6.72 (d, J=8.4 Hz, 1H), 5.22 (tt, J=4.1, 8.5 Hz, 1H), 4.60 (s, 2H), 3.87-3.80 (m, 1H), 3.60 (ddd, J=2.9, 9.0, 11.7 Hz, 2H), 3.43 (t, J=9.6 Hz, 2H), 1.82-1.72 (m, 2H), 1.58-1.52 (m, 2H).

### Step 3. 3-(6-(1-hydroxyethyl)-1-oxoisoindolin-2-yl)-1-((2-(trimethylsilyl)ethoxy)methyl)piperidine-2,6-dione

To a solution of 2-(2,6-dioxo-1-((2-(trimethylsilyl)ethoxy)methyl)piperidin-3-yl)-3-oxoisoindoline-5-carbaldehyde (1.00 g, 2.48 mmol, 1.00 eq) in dichloromethane (20.0 mL) was added methylmagnesium bromide (3.00 M, 994 uL, 1.20 eq) at −78° C. under nitrogen atmosphere. The reaction mixture was stirred at −78° C. for 2 h under nitrogen atmosphere. The mixture was quenched by ammonium chloride (30 mL) to pH=7 at 0° C. and extracted with ethyl acetate (3×50 mL). The combined organic layers were washed with brine (50 mL), dried over sodium sulfate, filtered and concentrated under reduced pressure to give a residue. The residue was purified by column chromatography (SiO2, petroleum ether/ethyl acetate=1/0 to 1/1) to afford 3-(6-(1-hydroxyethyl)-1-oxoisoindolin-2-yl)-1-((2-(trimethylsilyl)ethoxy)methyl)piperidine-2,6-dione (1.10 g, 2.63 mmol, 53% yield) as yellow oil.

1H NMR (400 MHz, DMSO-d6) δ=7.73 (s, 1H), 7.66-7.60 (m, 1H), 7.59-7.53 (m, 1H), 5.34 (d, J=4.4 Hz, 1H), 5.26 (dd, J=5.0, 13.4 Hz, 1H), 5.07 (q, J=9.7 Hz, 2H), 4.90-4.81 (m, 1H), 4.48 (d, J=17.0 Hz, 1H), 4.28 (d, J=17.0 Hz, 1H), 3.54 (dq, J=2.5, 8.0 Hz, 2H), 3.21-2.98 (m, 1H), 2.88-2.76 (m, 1H), 2.47-2.30 (m, 1H), 2.12-2.04 (m, 1H), 1.37 (d, J=6.5 Hz, 3H), 0.92-0.76 (m, 2H), 0.08-0.09 (m, 9H).

### Step 4. 3-(6-(1-azidoethyl)-1-oxoisoindolin-2-yl)-1-((2-(trimethylsilyl)ethoxy)methyl)piperidine-2,6-dione

To a solution of 3-(6-(1-hydroxyethyl)-1-oxoisoindolin-2-yl)-1-((2-(trimethylsilyl)ethoxy)methyl) piperidine-2,6-dione (500 mg, 1.19 mmol, 1.00 eq) in toluene (5.00 mL) was added diphenylphosphoryl azide (5.97 mmol, 1.29 mL, 5.00 eq) and 2,3,4,6,7,8,9,10-octahydropyrimido[1,2-a]azepine (5.97 mmol, 900 uL, 5.00 eq) at 0° C. under nitrogen atmosphere. The reaction mixture was stirred at 0° C. for 1 h under nitrogen atmosphere. The reaction mixture was stirred at 60° C. for 12 h under nitrogen atmosphere. The mixture was quenched by ammonium chloride (30 mL) to pH=7 at 0° C. and extracted with ethyl acetate (3×50 mL). The combined organic layers were washed with brine (50.0 mL), dried over sodium sulfate, filtered and concentrated under reduced pressure to give a residue. The residue was purified by column chromatography (SiO2, petroleum ether/ethyl acetate=1/0 to 1/1) to afford 3-(6-(1-azidoethyl)-1-oxoisoindolin-2-yl)-1-((2-(trimethylsilyl)ethoxy)methyl)piperidine-2,6-dione (500 mg, 1.13 mmol, 47% yield) as yellow oil.

1H NMR (400 MHz, DMSO-d6) δ=7.78 (s, 1H), 7.72-7.63 (m, 2H), 5.27 (dd, J=5.1, 13.4 Hz, 1H), 5.16-4.98 (m, 3H), 4.52 (d, J=17.2 Hz, 1H), 4.42-4.30 (m, 1H), 3.62-3.47 (m, 2H), 3.16-3.02 (m, 1H), 2.87-2.77 (m, 1H), 2.42 (br dd, J=4.3, 13.1 Hz, 1H), 2.15-2.03 (m, 1H), 1.52 (d, J=6.8 Hz, 3H), 0.91-0.73 (m, 2H), 0.13-0.20 (m, 9H).

### Step 5. 3-(6-(1-aminoethyl)-1-oxoisoindolin-2-yl)-1-((2-(trimethylsilyl)ethoxy)methyl)piperidine-2,6-dione

To a solution of 3-(6-(1-azidoethyl)-1-oxoisoindolin-2-yl)-1-((2-(trimethylsilyl)ethoxy)methyl)piperidine-2,6-dione (500 mg, 1.13 mmol, 1.00 eq) in ethyl acetate (5.00 mL) was added palladium on carbon (50.0 mg, 113 umol, 10% purity, 0.100 eq). The reaction mixture was stirred at 20° C. for 12 h under 15 psi of hydrogen atmosphere. The mixture was filtered and concentrated under reduced pressure to afford 3-(6-(1-aminoethyl)-1-oxoisoindolin-2-yl)-1-((2-(trimethylsilyl)ethoxy)methyl)piperidine-2,6-dione (470 mg, 1.13 mmol, 99% yield) as a black oil.

1H NMR (400 MHz, DMSO-d6) δ=7.79 (s, 1H), 7.65 (d, J=7.8 Hz, 1H), 7.54 (d, J=7.8 Hz, 1H), 5.26 (dd, J=5.0, 13.4 Hz, 1H), 5.07 (q, J=9.8 Hz, 2H), 4.46 (d, J=17.0 Hz, 1H), 4.34-4.23 (m, 1H), 4.14 (q, J=6.6 Hz, 1H), 3.60-3.53 (m, 2H), 3.10-3.04 (m, 1H), 2.81 (br dd, J=1.9, 15.5 Hz, 1H), 2.41 (br dd, J=4.4, 13.1 Hz, 1H), 2.13-2.02 (m, 1H), 1.30 (d, J=6.6 Hz, 3H), 0.89-0.82 (m, 2H), 0.00 (s, 9H).

### Step 6. (6-((tetrahydro-2H-pyran-4-yl)oxy)pyridin-3-yl)methyl (1-(2-(2,6-dioxo-1-((2-(trimethylsilyl)ethoxy)methyl)piperidin-3-yl)-3-oxoisoindolin-5-yl)ethyl)carbamate

To a solution of (6-((tetrahydro-2H-pyran-4-yl)oxy)pyridin-3-yl)methanol (50.0 mg, 239 umol, 1.00 eq) in tetrahydrofuran (1.00 mL) was added 1,1′-carbonyldiimidazole (77.5 mg, 478 umol, 2.00 eq) at 0° C. The reaction mixture was stirred at 20° C. for 1 h. The reaction mixture was added into a solution of 3-(6-(1-aminoethyl)-1-oxoisoindolin-2-yl)-1-((2-(trimethylsilyl)ethoxy)methyl)piperidine-2,6-dione (99.8 mg, 239 umol, 1.00 eq), 2,3,4,6,7,8,9,10-octahydropyrimido[1,2-a]azepine (36.4 mg, 239 umol, 36.0 uL, 1.00 eq) and triethylamine (239 umol, 33.3 uL, 1.00 eq) in tetrahydrofuran (1.00 mL) and dimethyformamide (1.00 mL). The reaction was stirred at 20° C. for 11 h. The mixture was filtered and concentrated under reduced pressure to give a crude product. The crude product was purified by reverse phase chromatography (C18, 80 g; condition: water/acetonitrile=100:0 to 0:100, 0.1% formic acid) and lyophilized to afford (6-((tetrahydro-2H-pyran-4-yl)oxy)pyridin-3-yl)methyl(1-(2-(2,6-dioxo-1-((2-(trimethylsilyl)ethoxy)methyl)piperidin-3-yl)-3-oxoisoindolin-5-yl)ethyl)carbamate (12.0 mg, 16.5 umol, 7% yield, 90% purity) as a white solid.

1H NMR (400 MHz, DMSO-d6) δ=8.16 (br s, 1H), 7.95 (br d, J=8.3 Hz, 1H), 7.79-7.68 (m, 2H), 7.62-7.52 (m, 2H), 6.81 (br d, J=8.4 Hz, 1H), 5.34-5.13 (m, 2H), 5.07 (q, J=9.7 Hz, 2H), 5.00-4.86 (m, 2H), 4.85-4.68 (m, 1H), 4.47 (br d, J=17.1 Hz, 1H), 4.28 (br d, J=16.9 Hz, 1H), 3.87 (td, J=4.2, 11.4 Hz, 2H), 3.57-3.47 (m, 4H), 3.16-3.01 (m, 1H), 2.82 (br d, J=16.9 Hz, 1H), 2.42 (br dd, J=3.7, 13.1 Hz, 1H), 2.12-1.91 (m, 3H), 1.71-1.54 (m, 2H), 1.38 (br d, J=6.5 Hz, 3H), 0.86 (br t, J=7.5 Hz, 2H), 0.00 (s, 9H).

### Step 7. (6-((tetrahydro-2H-pyran-4-yl)oxy)pyridin-3-yl)methyl (1-(2-(1-(hydroxymethyl)-2,6-dioxopiperidin-3-yl)-3-oxoisoindolin-5-yl)ethyl)carbamate

To a solution of (6-((tetrahydro-2H-pyran-4-yl)oxy)pyridin-3-yl)methyl(1-(2-(2,6-dioxo-1-((2-(trimethylsilyl) ethoxy)methyl)piperidin-3-yl)-3-oxoisoindolin-5-yl)ethyl)carbamate (10.0 mg, 15.3 umol, 1.00 eq) in dichloromethane (1.00 mL) was added trifluoroacetic acid (0.100 mL). The reaction mixture was stirred at 20° C. for 1 h. The reaction mixture was concentrated under reduced pressure to afford (6-((tetrahydro-2H-pyran-4-yl)oxy)pyridin-3-yl)methyl(1-(2-(1-(hydroxymethyl)-2,6-dioxopiperidin-3-yl)-3-oxoisoindolin-5-yl)ethyl)carbamate (10.0 mg, crude) as yellow oil.

### Step 8. (6-((tetrahydro-2H-pyran-4-yl)oxy)pyridin-3-yl)methyl (1-(2-(2,6-dioxopiperidin-3-yl)-3-oxoisoindolin-5-yl)ethyl)carbamate

To a solution of (6-((tetrahydro-2H-pyran-4-yl)oxy)pyridin-3-yl)methyl(1-(2-(1-(hydroxymethyl)-2,6-dioxopiperidin-3-yl)-3-oxoisoindolin-5-yl)ethyl)carbamate (10.0 mg, 18.1 umol, 1.00 eq) in acetonitrile (1.00 mL) was added ammonium hydroxide (0.0500 mL). The reaction mixture was stirred at 20° C. for 1 h. The reaction mixture was filtered. The filtrate was purified by Prep-HPLC (column: Phenomenex luna C18 150*25 mm*10 um; mobile phase: [water(formic acid)-acetonitrile]; B %: 12%-42%, 10.5 min) and lyophilized to afford (6-((tetrahydro-2H-pyran-4-yl)oxy)pyridin-3-yl)methyl (1-(2-(2,6-dioxopiperidin-3-yl)-3-oxoisoindolin-5-yl)ethyl)carbamate (4.47 mg, 8.38 umol, 46% yield, 98% purity) as a white solid.

1H NMR (400 MHz, DMSO-d6) δ=10.6 (br, 1H), 8.14 (s, 1H), 7.92 (br d, J=8.0 Hz, 1H), 7.69 (br s, 2H), 7.61-7.46 (m, 2H), 6.79 (br d, J=8.5 Hz, 1H), 5.22-5.07 (m, 2H), 5.00-4.85 (m, 2H), 4.81-4.70 (m, 1H), 4.48-4.38 (m, 1H), 4.34-4.24 (m, 1H), 3.85 (td, J=4.3, 11.7 Hz, 2H), 3.54-3.43 (m, 2H), 2.98-2.85 (m, 1H), 2.65-2.57 (m, 1H), 2.42-2.35 (m, 1H), 2.06-1.93 (m, 3H), 1.67-1.55 (m, 2H), 1.36 (br d, J=6.6 Hz, 3H).

### Example 17. Synthesis of Compound 17

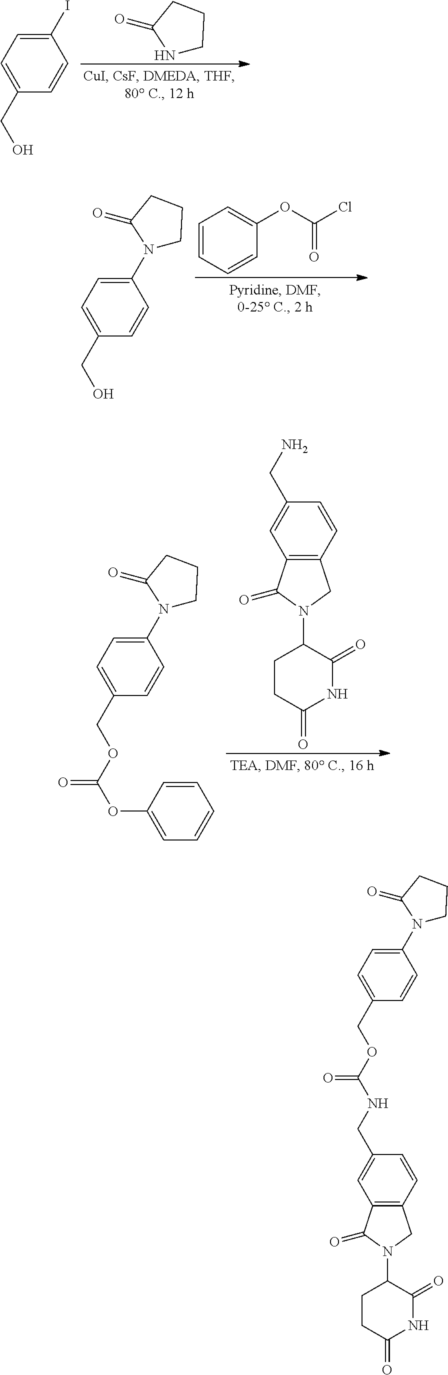

### Step 1. Synthesis of 1-(4-(hydroxymethyl)phenyl)-pyrrolidin-2-one

To a solution of (4-iodophenyl)methanol (2.00 g, 8.55 mmol, 1.00 eq) and pyrrolidin-2-one (800 mg, 9.40 mmol, 1.10 eq) in tetrahydrofuran (50 mL) were added N1,N2-dimethylethane-1,2-diamine (152 mg, 1.72 mmol, 0.20 eq), cesium fluoride (2.60 g, 17.1 mmol, 2.00 eq) and copper(I) iodide (160 mg, 840 μmol, 0.10 eq) under nitrogen atmosphere. After stirring at 80° C. for 12 h, the reaction mixture was cooled to room temperature. Water (30 mL) was added and layers were separated. The aqueous phase was extracted with ethyl acetate (20 mL). Combined extracts were washed with brine (30 mL), dried over sodium sulfate, filtered, and concentrated under vacuum. The residue was purified by flash silica gel chromatography (eluent of 0-100% ethyl acetate/petroleum ether) to afford 1-(4-(hydroxymethyl)phenyl)pyrrolidin-2-one (380 mg, 1.95 mmol, 23% yield) as a white solid.

1H NMR (400 MHz, DMSO-d6) δ=7.67-7.52 (m, 2H), 7.30 (d, J=8.8 Hz, 2H), 5.34-4.81 (m, 1H), 4.46 (s, 2H), 3.88-3.75 (m, 2H), 2.49-2.43 (m, 2H), 2.12-1.97 (m, 2H)

### Step 2. Synthesis of 4-(2-oxopyrrolidin-1-yl)benzyl phenyl carbonate

To a solution of 1-(4-(hydroxymethyl)phenyl)pyrrolidin-2-one (330 mg, 1.73 mmol, 1.00 eq) in dimethyl formamide (2 mL) were added pyridine (136 mg, 1.73 mmol, 1.00 eq) and phenyl carbonochloridate (540 mg, 3.45 mmol, 2.00 eq) at 0° C. The reaction mixture was stirred at 25° C. for 2 h. The residue was diluted with ethyl acetate (30 mL) and water (30 mL). The layers were separated and the aqueous phase was extracted with ethyl acetate (20 mL). The combined organic layers were dried over sodium sulfate, filtered and concentrated in vacuo. The residue was purified by flash silica gel chromatography (ISCO®; 12 g SepaFlash® Silica Flash Column, Eluent of 0-80% Ethyl acetate/Petroleum ether gradient @40 mL/min) to afford 4-(2-oxopyrrolidin-1-yl)benzyl phenyl carbonate (400 mg, 1.26 mmol, 72% yield) as a white solid. MS (ESI) m/z 312.2 [M+H]+

### Step 3. Synthesis of 4-(2-oxopyrrolidin-1-yl)benzyl ((2-(2,6-dioxopiperidin-3-yl)-3-oxoisoindolin-5-yl)methyl)carbamate

To a solution of 4-(2-oxopyrrolidin-1-yl)benzyl phenyl carbonate (270 mg, 658 μmol, 76% purity, 1.20 eq) and 3-(6-(aminomethyl)-1-oxoisoindolin-2-yl)piperidine-2,6-dione (150 mg, 548 μmol, 1.00 eq) in dimethyl formamide (3 mL) was added triethylamine (166 mg, 1.65 mmol, 3.00 eq). The reaction mixture was stirred at 80° C. for 16 h. The mixture was filtered and the filtrate was purified by Prep-HPLC (column: YMC Triart 30*150 mm*7 μm; mobile phase: [water (hydrochloric acid)-acetonitrile]; B %: 24%-44%, 7 min) and lyophilized to give 4-(2-oxopyrrolidin-1-yl)benzyl ((2-(2,6-dioxopiperidin-3-yl)-3-oxoisoindolin-5-yl)methyl)carbamate (25.56 mg, 51.5 μmol, 9.0% yield) as a white solid.

1H NMR (400 MHz, DMSO-d6) δ=10.98 (s, 1H), 7.95-7.86 (m, 1H), 7.68-7.59 (m, 3H), 7.53 (q, J=8.0 Hz, 2H), 7.35 (d, J=8.4 Hz, 2H), 5.11 (dd, J=5.2, 13.2 Hz, 1H), 5.01 (s, 2H), 4.48-4.39 (m, 1H), 4.34-4.26 (m, 3H), 3.82 (t, J=7.2 Hz, 2H), 2.96-2.86 (m, 1H), 2.60 (br d, J=18.4 Hz, 2H), 2.44-2.37 (m, 2H), 2.10-1.96 (m, 3H). MS (ESI) m/z 491.2 [M+H]+.

### Example 18. Synthesis of Compound 18

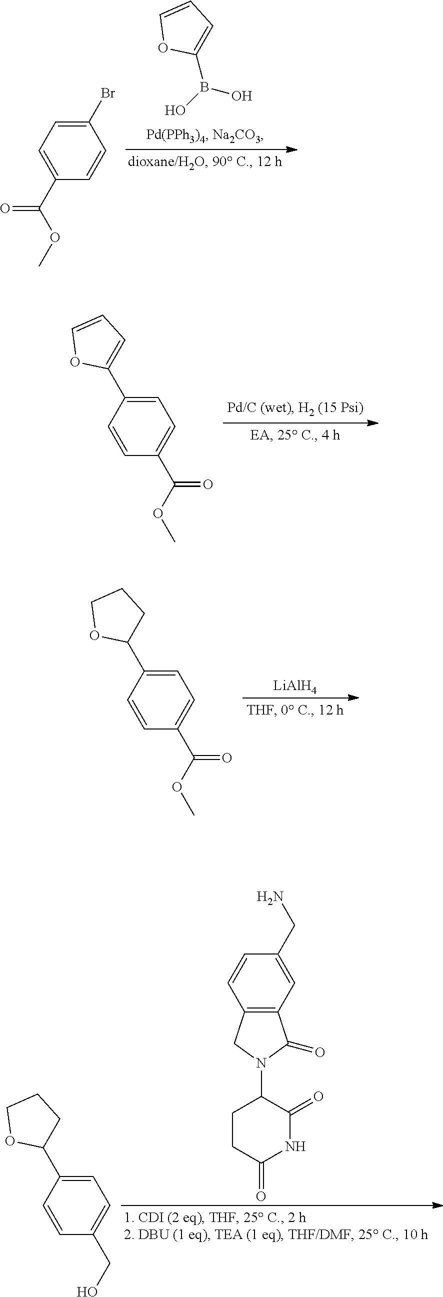

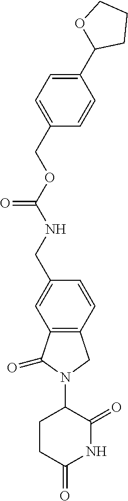 [SMILES: O=C1CCC(N2Cc3ccc(CNC(=O)OCc4ccc(C5CCCO5)cc4)cc3C2=O)C(=O)N1]

### Step 1. Synthesis of methyl 4-(furan-2-yl)benzoate

A mixture of methyl 4-bromobenzoate (1.50 g, 6.98 mmol, 1.00 eq), furan-2-ylboronic acid (937 mg, 8.37 mmol, 1.20 eq), sodium carbonate (2.22 g, 20.9 mmol, 3.00 eq) and tetratriphenylphosphine palladium (403 mg, 349 μmol, 0.0500 eq) in dioxane (20.0 mL) and water (4.00 mL) was degassed and purged with nitrogen for 3 times, and then the mixture was stirred at 90° C. for 12 h under nitrogen atmosphere. The reaction mixture was quenched with water (30 mL) and then extracted with ethyl acetate (3×30 mL). The organic phase was washed with brine (3×40 mL), dried over anhydrous sodium sulfate, filtered and concentrated under reduced pressure to give a residue. The residue was purified by flash silica gel chromatography (ISCO®; 20 g SepaFlash® Silica Flash Column, Eluent of 0-15% Ethyl acetate/Petroleum ether gradient @ 30 mL/min) to afford methyl 4-(furan-2-yl)benzoate (600 mg, 2.97 mmol, 42% yield) as light yellow solid.

1H NMR (400 MHz, DMSO-d6) δ=7.97-8.02 (m, 2H), 7.80-7.86 (m, 3H), 7.16 (d, J=2.9 Hz, 1H), 6.66 (dd, J=3.4, 1.8 Hz, 1H), 3.86 (s, 3H). MS (ESI) m/z 203.1 [M+H]+.

### Step 2. Synthesis of methyl 4-(tetrahydrofuran-2-yl)benzoate

To a flask was added methyl 4-(furan-2-yl)benzoate (350 mg, 1.73 mmol, 1.00 eq) and ethyl acetate (30.0 mL) and the mixture was degassed and purged with nitrogen for 3 times. Palladium on carbon (210 mg, 10.0% purity) was added to the mixture under nitrogen atmosphere and the mixture was degassed and purged with hydrogen for 3 times. The reaction mixture was stirred at 25° C. for 4 h under hydrogen atmosphere (15 Psi). The reaction mixture was filtered and washed with ethyl acetate (30 mL). The filtrate was concentrated under reduced pressure to give a residue. The residue was purified by flash silica gel chromatography (ISCO®; 4 g SepaFlash® Silica Flash Column, Eluent of 0-15% Ethyl acetate/Petroleum ether gradient @30 mL/min) to afford methyl 4-(tetrahydrofuran-2-yl)benzoate (300 mg, 1.45 mmol, 84% yield) as colorless oil.

1H NMR (400 MHz, DMSO-d6) δ=7.92 (d, J=8.3 Hz, 2H), 7.44 (d, J=8.2 Hz, 2H), 4.87 (t, J=7.2 Hz, 1H), 3.96-4.03 (m, 1H), 3.79-3.86 (m, 4H), 2.27-2.38 (m, 1H), 1.87-1.97 (m, 2H), 1.62 (dq, J=12.1, 7.9 Hz, 1H).

### Step 3. Synthesis of (4-(tetrahydrofuran-2-yl)phenyl)methanol

To a flask was added lithium aluminum hydride (55.2 mg, 1.49 mmol, 2.00 eq) and it was degassed and purged with nitrogen for 3 times. Then tetrahydrofuran (6.00 mL) was added dropwise to the flask and the mixture was stirred at 0° C. for 30 min. A solution of methyl 4-(tetrahydrofuran-2-yl)benzoate (150 mg, 727 μmol, 1.00 eq) in tetrahydrofuran (6.00 mL) was added dropwise to the mixture and the mixture was stirred at 0° C. for 12 h. The reaction mixture was quenched with sodium sulfate decahydrate at 25° C. and then extracted with ethyl acetate (3×5 mL). The organic phase was washed with brine (3×10 mL), dried over anhydrous sodium sulfate, filtered and concentrated under reduced pressure to afford (4-(tetrahydrofuran-2-yl)phenyl)methanol (100 mg, crude) as a light yellow oil. It was used for next step without further purification.

1H NMR (400 MHz, DMSO-d6) δ=7.26 (s, 4H), 5.12 (t, J=5.8 Hz, 1H), 4.77 (t, J=7.2 Hz, 1H), 4.46 (d, J=5.8 Hz, 2H), 3.93-4.06 (m, 1H), 3.74-3.85 (m, 1H), 2.26 (dq, J=12.5, 6.4 Hz, 1H), 1.88-1.97 (m, 2H), 1.59-1.68 (m, 1H). MS (ESI) m/z 161.2 [M-OH]+.

### Example 19. Synthesis of Compound 19

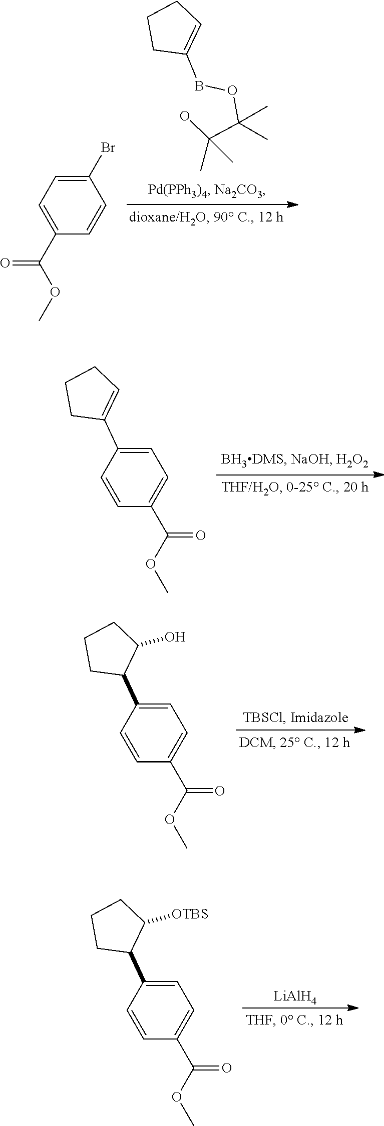

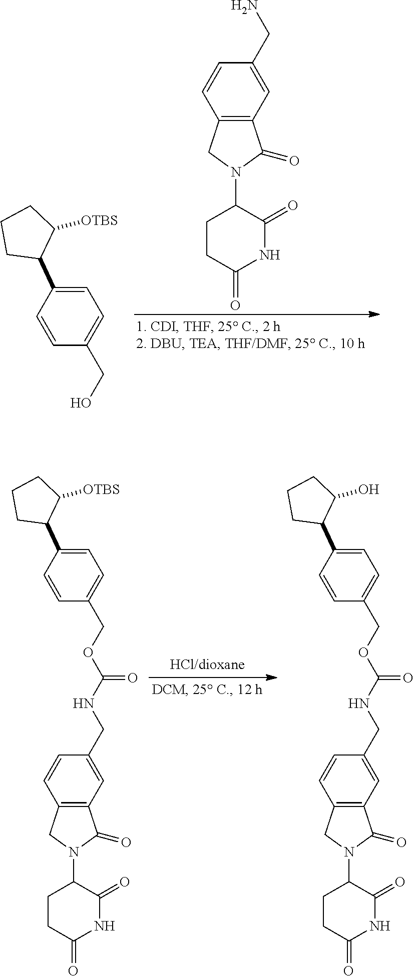 [SMILES: CC(C)(C)[Si](C)(C)O[C@H]1CCC[C@@H]1c1ccc(CO)cc1 ; NCc1ccc2c(c1)C(=O)N(C1CCC(=O)NC1=O)C2 ; CC(C)(C)[Si](C)(C)O[C@H]1CCC[C@@H]1c1ccc(COC(=O)NCc2ccc3c(c2)C(=O)N(C2CCC(=O)NC2=O)C3)cc1 ; O=C1CCC(N2Cc3ccc(CNC(=O)OCc4ccc([C@H]5CCC[C@@H]5O)cc4)cc3C2=O)C(=O)N1]

### Step 1. Synthesis of methyl 4-(cyclopent-1-en-1-yl) benzoate

A mixture of methyl 4-bromobenzoate (1.50 g, 6.98 mmol, 1.00 eq), 2-(cyclopent-1-en-1-yl)-4,4,5,5-tetramethyl-1,3,2-dioxaborolane (1.62 g, 8.37 mmol, 1.20 eq), sodium carbonate (4.44 g, 41.8 mmol, 6.00 eq) and tetratriphenylphosphine palladium (403 mg, 349 μmol, 0.0500 eq) in dioxane (25.0 mL) and water (3.00 mL) was degassed and purged with nitrogen for 3 times, and then the mixture was stirred at 90° C. for 12 h under nitrogen atmosphere. The reaction mixture was quenched with water (20 mL) and then extracted with ethyl acetate (3×15 mL). The organic phase was washed with brine (3×20 mL), dried over anhydrous sodium sulfate, filtered and concentrated under reduced pressure to give a residue. The residue was purified by flash silica gel chromatography (ISCO®; 20 g SepaFlash® Silica Flash Column, Eluent of 0˜5% Ethyl acetate/Petroleum ether gradient @ 40 mL/min) to afford methyl 4-(cyclopent-1-en-1-yl) benzoate (930 mg, 4.60 mmol, 66% yield) as a light-yellow solid.

1H NMR (400 MHz, CDCl3) δ=7.98 (d, J=8.2 Hz, 2H), 7.48 (d, J=8.2 Hz, 2H), 6.34 (br s, 1H), 3.92 (s, 3H), 2.69-2.77 (m, 2H), 2.52-2.61 (m, 2H), 2.05 (quin, J=7.5 Hz, 2H). MS (ESI) m/z 203.2 [M+H]+.

### Step 2. Synthesis of methyl 4-((1R, 2S)-2-hydroxycyclopentyl) benzoate

To a solution of methyl 4-(cyclopent-1-en-1-yl) benzoate (1.20 g, 5.93 mmol, 1.00 eq) in tetrahydrofuran (15.0 mL) was added dropwise borane dimethyl sulfide complex (10.0 M, 1.19 mL, 2.00 eq) at 0° C. under nitrogen atmosphere and the mixture was stirred at 25° C. for 15 h under nitrogen atmosphere. Then the reaction mixture was cooled to 0° C., hydrogen peroxide (35.0% purity, 11.8 mL, 24.2 eq) was added dropwise to the mixture, followed by addition of aqueous sodium hydroxide (2.00 M, 11.9 mL, 4.00 eq), then the mixture was stirred at 25° C. for 5 h. The reaction mixture was quenched with saturated sodium sulfite (20 mL) and then extracted with ethyl acetate (3×15 mL). The organic phase was washed with brine (3×20 mL), dried over anhydrous sodium sulfate, filtered and concentrated under reduced pressure to give a residue. The residue was purified by flash silica gel chromatography (ISCO®; 20 g SepaFlash® Silica Flash Column, Eluent of 0-30% Ethyl acetate/Petroleum ether gradient @ 20 mL/min) to afford methyl 4-((1R, 2S)-2-hydroxycyclopentyl) benzoate (580 mg, 2.63 mmol, 44% yield) as colorless oil.

1H NMR (400 MHz, DMSO-d6) δ=7.84-7.91 (m, 2H), 7.40 (d, J=8.3 Hz, 2H), 4.80 (d, J=5.4 Hz, 1H), 3.93-4.06 (m, 1H), 3.83 (s, 3H), 2.80-2.92 (m, 1H), 2.02-2.11 (m, 1H), 1.90-1.98 (m, 1H), 1.50-1.81 (m, 4H). MS (ESI) m/z 203.0 [M+H]+

### Step 3. Synthesis of methyl 4-((1R, 2S)-2-((tert-butyldimethylsilyl) oxy) cyclopentyl) benzoate

To a solution of methyl 4-((1R,2S)-2-hydroxycyclopentyl)benzoate (560 mg, 2.54 mmol, 1.00 eq) in dichloromethane (15.0 mL) was added imidazole (519 mg, 7.63 mmol, 3.00 eq) and tert-butylchlorodimethylsilane (1.15 g, 7.63 mmol, 3.00 eq). The mixture was stirred at 25° C. for 12 h. The reaction mixture was quenched with water (30 mL) and then extracted with ethyl acetate (3×20 mL). The organic phase was washed with brine (3×30 mL), dried over anhydrous sodium sulfate, filtered and concentrated under reduced pressure to give a residue. The residue was purified by flash silica gel chromatography (ISCO®; 12 g SepaFlash® Silica Flash Column, Eluent of 0-20% Ethyl acetate/Petroleum ether gradient @ 30 mL/min) to afford methyl 4-((1R, 2S)-2-((tert-butyldimethylsilyl) oxy) cyclopentyl) benzoate (800 mg, 2.37 mmol, 93% yield) as colorless oil.

1H NMR (400 MHz, DMSO-d6) δ=7.88 (d, J=8.2 Hz, 2H), 7.38 (d, J=8.2 Hz, 2H), 3.83 (s, 3H), 2.82-2.92 (m, 1H), 1.92-2.06 (m, 2H), 1.69-1.81 (m, 3H), 1.47-1.62 (m, 1H), 0.72 (s, 9H), −0.20 (s, 3H), −0.33 (s, 3H). MS (ESI) m/z 335.3 [M+H]+

### Step 4. Synthesis of (4-((1R, 2S)-2-((tert-butyldimethylsilyl) oxy) cyclopentyl)phenyl)methanol

To a flask was added lithium aluminum hydride (159 mg, 4.19 mmol, 2.00 eq) and it was degassed and purged with nitrogen for 3 times. Then tetrahydrofuran (8.00 mL) was added dropwise to the flask and the mixture was stirred at 0° C. for 30 min. A solution of methyl 4-((1R, 2S)-2-((tert-butyldimethylsilyl) oxy)cyclopentyl)benzoate (700 mg, 2.09 mmol, 1.00 eq) in tetrahydrofuran (8.00 mL) was added dropwise to the mixture and the mixture was stirred at 0° C. for 12 h. The reaction mixture was quenched with sodium sulfate decahydrate at 25° C. and then extracted with ethyl acetate (3×15 mL). The organic phase was washed with brine (3×30 mL), dried over anhydrous sodium sulfate, filtered and concentrated under reduced pressure to give a residue. The residue was purified by flash silica gel chromatography (ISCO®; 12 g SepaFlash® Silica Flash Column, Eluent of 0-30% Ethyl acetate/Petroleum ether gradient @ 30 mL/min) to afford (4-((1R, 2S)-2-((tert-butyldimethylsilyl) oxy) cyclopentyl) phenyl) methanol (567 mg, 1.85 mmol, 77% yield) as colorless oil.

1H NMR (400 MHz, DMSO-d6) δ=7.19-7.23 (m, 2H), 7.15-7.18 (m, 2H), 5.11 (t, J=5.7 Hz, 1H), 4.45 (d, J=5.8 Hz, 2H), 4.04 (q, J=7.0 Hz, 1H), 2.73-2.82 (m, 1H), 1.92-2.03 (m, 2H), 1.62-1.78 (m, 3H), 1.48-1.60 (m, 1H), 0.75 (s, 9H), −0.18 (s, 3H), −0.27 (s, 3H). MS (ESI) m/z 289.2 [M+H]+.

### Step 5. Synthesis of 4-((1R, 2S)-2-((tert-butyldimethylsilyl) oxy) cyclopentyl)benzyl ((2-(2,6-dioxopiperidin-3-yl)-3-oxoisoindolin-5-yl)methyl)carbamate

To a solution of (4-((1R, 2S)-2-((tert-butyldimethylsilyl) oxy) cyclopentyl) phenyl) methanol (60.0 mg, 196 μmol, 1.00 eq) in tetrahydrofuran (1.00 mL) was added di(1H-imidazol-1-yl)methanone (63.5 mg, 392 μmol, 2.00 eq). The mixture was stirred at 25° C. for 2 h. Then the resulting solution was added to a suspension of 3-(6-(aminomethyl)-1-oxoisoindolin-2-yl)piperidine-2,6-dione (53.5 mg, 196 μmol, 1.00 eq), trimethylamine (19.8 mg, 196 μmol, 27.3 μL, 1.00 eq) and 3,4,5,7,8,9,10,10a-octahydropyrido[1,2-a][1,4]diazepine (29.8 mg, 196 μmol, 29.5 μL, 1.00 eq) in tetrahydrofuran (1.00 mL) and dimethylformamide (0.500 mL). The mixture was stirred at 25° C. for 10 h. The reaction mixture was quenched with water (5 mL) and then extracted with ethyl acetate (3×5 mL). The organic phase was washed with brine (3×10 mL), dried over anhydrous sodium sulfate, filtered and concentrated under reduced pressure to give a residue. The residue was purified by Perp-TLC (silicon dioxide, Petroleum ether:Ethyl acetate=1:1) to afford 4-((1R, 2S)-2-((tert-butyldimethylsilyl) oxy) cyclopentyl)benzyl ((2-(2,6-dioxopiperidin-3-yl)-3-oxoisoindolin-5-yl)methyl)carbamate (35 mg, 57.8 μmol, 29% yield) as a white solid.

1H NMR (400 MHz, DMSO-d6) δ=10.99 (s, 1H), 7.89 (t, J=6.1 Hz, 1H), 7.63 (s, 1H), 7.49-7.57 (m, 2H), 7.25-7.30 (m, 2H), 7.20-7.24 (m, 2H), 5.11 (dd, J=13.2, 5.1 Hz, 1H), 5.01 (s, 2H), 4.40-4.46 (m, 1H), 4.27-4.34 (m, 3H), 4.01-4.09 (m, 1H), 3.30 (s, 1H), 2.85-2.97 (m, 1H), 2.73-2.84 (m, 1H), 1.92-2.05 (m, 4H), 1.62-1.80 (m, 3H), 1.49-1.60 (m, 1H), 0.74 (s, 9H), −0.18 (s, 3H), −0.27 (s, 3H). MS (ESI) m/z 606.4 [M+H]+.

### Step 6. Synthesis of 4-((1R, 2S)-2-hydroxycyclopentyl) benzyl ((2-(2,6-dioxopiperidin-3-yl)-3-oxoisoindolin-5-yl)methyl)carbamate

To a solution of 4-((1R, 2S)-2-((tert-butyldimethylsilyl) oxy)cyclopentyl)benzyl ((2-(2,6-dioxopiperidin-3-yl)-3-oxoisoindolin-5-yl)methyl)carbamate (70.0 mg, 116 μmol, 1.00 eq) in dichloromethane (2.00 mL) was added hydrochloric acid (4 M in dioxane, 231 μL, 8.00 eq). The mixture was stirred at 25° C. for 12 h. The reaction mixture was quenched with water (10 mL) and then extracted with ethyl acetate (3×10 mL). The organic phase was washed with brine (3×20 mL), dried over anhydrous sodium sulfate, filtered and concentrated under reduced pressure to give a residue. The residue was purified by Prep-HPLC (FA condition; column: Phenomenex luna C18 150*25 mm*10 μm; mobile phase: [water (formic acid)-acetonitrile]; B %: 3%-33%, 10 min) to afford 4-((1R,2S)-2-hydroxycyclopentyl) benzyl ((2-(2,6-dioxopiperidin-3-yl)-3-oxoisoindolin-5-yl)methyl)carbamate (9.21 mg, 18.74 μmol, 16% yield) as an off-white solid.

1H NMR (400 MHz, DMSO-d6) δ=10.98 (s, 1H), 7.88 (br t, J=5.7 Hz, 1H), 7.63 (s, 1H), 7.49-7.57 (m, 2H), 7.25 (q, J=7.7 Hz, 4H), 5.11 (br dd, J=13.4, 4.9 Hz, 1H), 5.00 (s, 2H), 4.70 (d, J=5.2 Hz, 1H), 4.40-4.47 (m, 1H), 4.27-4.33 (m, 3H), 3.95 (dt, J=12.6, 6.5 Hz, 1H), 2.85-2.97 (m, 1H), 2.73-2.83 (m, 1H), 2.62 (br s, 1H), 2.38-2.42 (m, 1H), 1.97-2.04 (m, 2H), 1.92 (br dd, J=13.0, 6.3 Hz, 1H), 1.55-1.74 (m, 4H).

1H NMR (400 MHz, CD3OD) δ=7.74 (s, 1H), 7.50-7.60 (m, 2H), 7.09-7.34 (m, 4H), 5.15 (br dd, J=13.2, 5.0 Hz, 1H), 5.08 (s, 2H), 4.47 (br d, J=7.2 Hz, 2H), 4.40 (s, 2H), 4.11 (q, J=6.8 Hz, 1H), 2.75-2.96 (m, 3H), 2.41-2.57 (m, 1H), 2.01-2.21 (m, 3H), 1.64-1.88 (m, 4H). MS (ESI) m/z 492.2 [M+H]+.

### Example 20. Synthesis of Compound 20

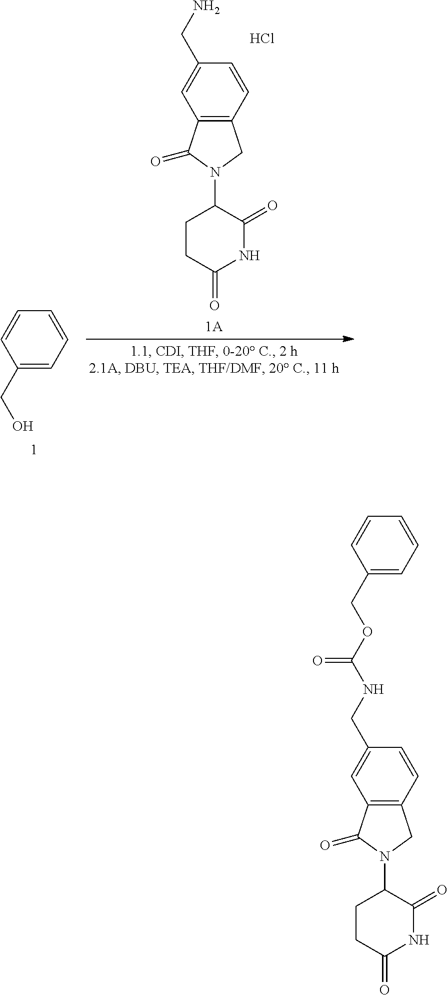 [SMILES: NCc1ccc2c(c1)C(=O)N(C1CCC(=O)NC1=O)C2 ; Cl ; OCc1ccccc1 ; O=C1CCC(N2Cc3ccc(CNC(=O)OCc4ccccc4)cc3C2=O)C(=O)N1]

### Step 1. Synthesis of benzyl ((2-(2,6-dioxopiperidin-3-yl)-3-oxoisoindolin-5-yl)methyl)carbamate

To a solution of phenylmethanol (50.0 mg, 462 umol, 48.0 uL, 1.00 eq) in tetrahydrofuran (0.500 mL) was added di(1H-imidazol-1-yl)methanone (149 mg, 924 umol, 2.00 eq) at 0° C. The mixture was stirred at 20° C. for 2 h. Then the resulting solution was added to a suspension of 3-(6-(aminomethyl)-1-oxoisoindolin-2-yl)piperidine-2,6-dione (143 mg, 462 umol, 1.00 eq, hydrochloride), triethylamine (46.7 mg, 462 umol, 64.3 uL, 1.00 eq), 3,4,5,7,8,9,10,10a-octahydropyrido[1,2-a][1,4]diazepine (70.3 mg, 462 umol, 69.6 uL, 1.00 eq) in tetrahydrofuran (0.500 mL). The mixture was stirred at 20° C. for 1 h. After adding dimethylformamide (0.500 mL), the mixture was stirred at 20° C. for 10 h. The mixture was concentrated under reduced pressure to remove tetrahydrofuran. The mixture was filtered and the filtrate was purified by Prep-HPLC (Phenomenex Synergi C18 150*25 mm*10 um; mobile phase: [water (formic acid)-acetonitrile]; B %: 20%-50%, 10 min) and lyophilized to afford benzyl ((2-(2,6-dioxopiperidin-3-yl)-3-oxoisoindolin-5-yl)methyl)carbamate (115.07 mg, 279.61 umol, 60% yield 99% purity) as a white solid.

1H NMR (400 MHz, DMSO-d6) δ=10.99 (s, 1H), 7.95 (br t, J=6.1 Hz, 1H), 7.63 (s, 1H), 7.54 (q, J=7.4 Hz, 2H), 7.40-7.13 (m, 5H), 5.12 (dd, J=5.0, 13.3 Hz, 1H), 5.05 (s, 2H), 4.47-4.40 (m, 1H), 4.34-4.27 (m, 3H), 2.97-2.86 (m, 1H), 2.60 (td, J=1.8, 15.3 Hz, 1H), 2.40 (br dd, J=4.5, 13.1 Hz, 1H), 2.04-1.96 (m, 1H). MS (ESI) m/z 408.1, 815.4 [M+H, 2M+H]+.

### Example 21. Synthesis of Compound 21

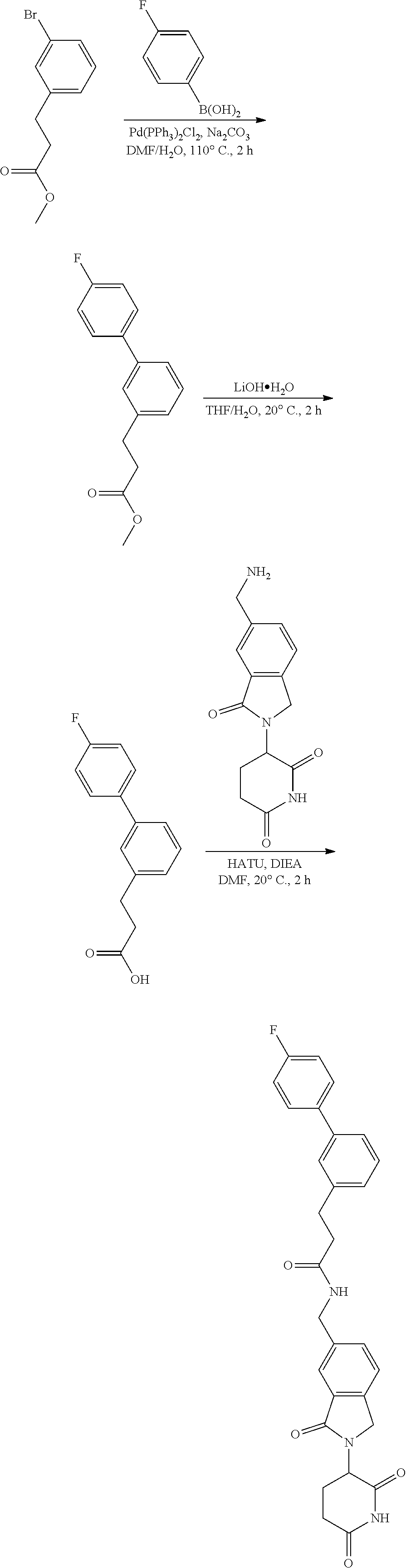

### Step 1. Synthesis of methyl 3-(4′-fluoro-[1,1′-biphenyl]-3-yl)propanoate

To a solution of methyl 3-(3-bromophenyl)propanoate (1.00 g, 4.11 mmol, 1.00 eq) in dimethylformamide (10.0 mL) and water (1.25 mL) were added sodium carbonate (1.09 g, 10.3 mmol, 2.50 eq), bis(triphenylphosphine)palladium(II) chloride (578 mg, 823 umol, 0.200 eq) and (4-fluorophenyl)boronic acid (691 mg, 4.94 mmol, 1.20 eq). The reaction mixture was stirred at 110° C. for 2 h under nitrogen atmosphere. The reaction mixture was poured into water (100 mL). The aqueous phase was extracted with ethyl acetate (2×40 mL). The combined organic layers were washed with brine (100 mL), dried over anhydrous sodium sulfate, filtered and concentrated under reduced pressure to give a residue. The residue was purified by column chromatography (SiO2, petroleum ether/ethyl acetate=1/0 to 50/1) to afford methyl 3-(4′-fluoro-[1,1′-biphenyl]-3-yl)propanoate (590 mg, 2.28 mmol, 56% yield) as yellow oil.

1H NMR (400 MHz, DMSO-d6) δ=7.72-7.66 (m, 2H), 7.51 (s, 1H), 7.46 (d, J=7.8 Hz, 1H), 7.36 (t, J=7.7 Hz, 1H), 7.31-7.26 (m, 2H), 7.22 (d, J=7.7 Hz, 1H), 3.59 (s, 3H), 2.95-2.88 (m, 2H), 2.73-2.67 (m, 2H).

### Step 2. Synthesis of 3-(4′-fluoro-[1,1′-biphenyl]-3-yl)propanoic acid

To a solution of methyl 3-(4′-fluoro-[1,1′-biphenyl]-3-yl)propanoate (590 mg, 2.28 mmol, 1.00 eq) in tetrahydrofuran (6.00 mL) and water (2.00 mL) was added lithium hydroxide monohydrate (288 mg, 6.85 mmol, 3.00 eq). The reaction mixture was stirred at 20° C. for 2 h. The reaction mixture was added 1M hydrochloric acid until PH<7 and then extracted with ethyl acetate (2×20 mL). The organic layers were dired over anhydrous sodium sulfate, filtered and concentrated under reduced pressure to afford 3-(4′-fluoro-[1,1′-biphenyl]-3-yl)propanoic acid (220 mg, 901 umol, 40% yield) as a yellow solid.

1H NMR (400 MHz, DMSO-d6) δ=12.15 (br s, 1H), 7.69 (dd, J=5.6, 7.7 Hz, 2H), 7.50 (s, 1H), 7.45 (br d, J=7.7 Hz, 1H), 7.36 (t, J=7.6 Hz, 1H), 7.28 (t, J=8.5 Hz, 2H), 7.22 (br d, J=7.4 Hz, 1H), 2.89 (t, J=7.6 Hz, 2H), 2.59 (t, J=7.6 Hz, 2H)

### Step 3. Synthesis of N-((2-(2,6-dioxopiperidin-3-yl)-3-oxoisoindolin-5-yl)methyl)-3-(4′-fluoro-[1,1′-biphenyl]-3-yl)propanamide

To a solution of 3-(4′-fluoro-[1,1′-biphenyl]-3-yl)propanoic acid (23.7 mg, 96.9 umol, 1.00 eq) in dimethylformamide (0.500 mL) were added N,N-diisopropylethylamine (27.5 mg, 213 umol, 37.1 uL, 2.20 eq) and 2-(3H-[1,2,3]triazolo[4,5-b]pyridin-3-yl)-1,1,3,3-tetramethyluroniumhexafluorophosphate(V) (44.2 mg, 116. umol, 1.20 eq). The mixture was stirred at 20° C. for 0.5 h. Then 3-(6-(aminomethyl)-1-oxoisoindolin-2-yl)piperidine-2,6-dione (30.0 mg, 96.9 umol, 1.00 eq, hydrochloride) was added to the mixture and the reaction mixture was stirred at 20° C. for 1.5 h. The reaction mixture was filtered. The filtrate was purified by Prep-HPLC(column: Phenomenex Synergi C18 150*25 mm*10 um; mobile phase: [water(formic acid)-acetonitrile]; B %: 38%-58%, 10 min) and lyophilized to afford N-((2-(2,6-dioxopiperidin-3-yl)-3-oxoisoindolin-5-yl)methyl)-3-(4′-fluoro-[1,1′-biphenyl]-3-yl)propanamide (21.67 mg, 42.95 umol, 44% yield, 99% purity) as an off-white solid.

1H NMR (400 MHz, DMSO-d6) δ=10.98 (br s, 1H), 8.44 (br t, J=5.8 Hz, 1H), 7.66 (br dd, J=5.7, 7.4 Hz, 2H), 7.59 (s, 1H), 7.51-7.42 (m, 3H), 7.41-7.32 (m, 2H), 7.27 (t, J=8.4 Hz, 2H), 7.20 (br d, J=7.2 Hz, 1H), 5.18-5.06 (m, 1H), 4.44-4.23 (m, 4H), 2.97-2.85 (m, 3H), 2.63-2.57 (m, 1H), 2.55-2.52 (m, 2H), 2.45-2.35 (m, 1H), 1.99 (br dd, J=5.5, 6.5 Hz, 1H). MS (ESI) m/z 500.2 [M+H]+.

The following experimental procedures were used to prepare the compounds of Example 22 below.

### Synthetic Procedure 1

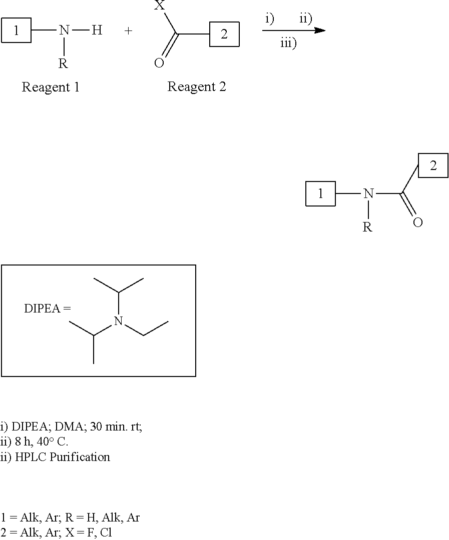

Reagent 1 (appr. 1 eq.), and Reagent 2 (appr. 1.3 eq.), and Diisopropylethylamine (DIPEA) (appr. 3.5 eq.) were mixed in dry Dimethylacetamide (DMA) (appr. 0.7 ml per 100 mg of product). The reaction mixture was sealed and heated for 8 hours at 40°, then cooled to the ambient temperature, and the solvent was evaporated under reduced pressure. The residue was dissolved in the DMSO (appr. 1 ml up to 300 mg of product) and DMSO solution was filtered, analyzed by LCMS, and transferred for HPLC purification.

### Synthetic Procedure 2

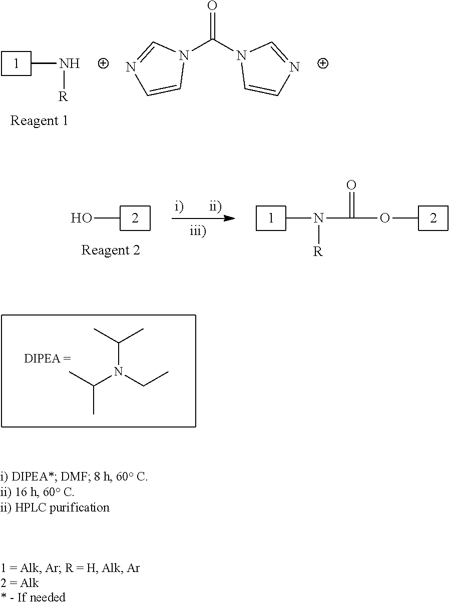

Reagent 2 (appr. 1.3-2 eq.) and 1,1′-Carbonyldiimidazole (CDI) (appr. 1.8-2.5 eq.) were mixed in dry Dimethylformamide*(DMF) (appr. 0.7 ml per 100 mg of product). The reaction mixture was sealed and heated for 8 hours at 60° C. Then the mixture was cooled to the ambient temperature and Reagent 1 (appr. 1 eq.) was added in one portion. The reaction mixture was sealed and heated for 16 hours at 60° C., and cooled to the ambient temperature. The solvent was evaporated under reduced pressure and the residue was dissolved in the DMSO (appr. 1 ml up to 300 mg of product). DMSO solution was filtered, analyzed by LCMS, and transferred for HPLC purification.

### Example 22. Preparation of Compounds 22-66

2-phenylethyl N-{[2-(2,6-dioxopiperidin-3-yl)-3-oxo-2,3-dihydro-1H-isoindol-5-yl]methyl}carbamate (Compound 22) was obtained by Synthetic procedure 1 with using 40 mg (0.129 mmol) of 3-[6-(aminomethyl)-1-oxo-2,3-dihydro-1H-isoindol-2-yl]piperidine-2,6-dione hydrochloride (Reagent 1), 29 mg (0.157 mmol) of 2-phenylethyl carbonochloridate (Reagent 2), and 54.7 mg (0.423 mmol) of DIPEA. Purified by HPLC (gradient: from A-80%: B-20% to A-55%: B-45%; Rf=0.73; run time=6.5 min). Yield: 29.7 mg (58.3%). Yellow powder. LCMS purity: 98.3% (LCMS, Rf=0.44, run time=2.5 min). EI MS m/z: pos. 422.0 (MH+).

3-phenylpropyl N-{[2-(2,6-dioxopiperidin-3-yl)-3-oxo-2,3-dihydro-1H-isoindol-5-yl]methyl}carbamate (Compound 23) was obtained by Synthetic procedure 1 with using 41 mg (0.132 mmol) of 3-[6-(aminomethyl)-1-oxo-2,3-dihydro-1H-isoindol-2-yl]piperidine-2,6-dione hydrochloride (Reagent 1), 30 mg (0.151 mmol) of 3-phenylpropyl carbonochloridate (Reagent 2), and 52.6 mg (0.407 mmol) of DIPEA. Purified by HPLC (gradient: from A-75%: B-25% to A-50%: B-50%; Rf=0.71; run time=6.5 min). Yield: 28.7 mg (56.7%). Beige powder. LCMS purity: 100% (LCMS, Rf=0.58, run time=2 min). EI MS m/z: pos. 436.2 (MH+).

(4-chlorophenyl)methyl N-{[2-(2,6-dioxopiperidin-3-yl)-3-oxo-2,3-dihydro-1H-isoindol-5-yl]methyl}carbamate (Compound 24) was obtained by Synthetic procedure 1 with using 40 mg (0.129 mmol) of 3-[6-(aminomethyl)-1-oxo-2,3-dihydro-1H-isoindol-2-yl]piperidine-2,6-dione hydrochloride (Reagent 1), 32 mg (0.156 mmol) of (4-chlorophenyl)methyl carbonochloridate (Reagent 2), and 54.3 mg (0.42 mmol) of DIPEA. Purified by HPLC (gradient: from A-85%: B-15% to A-60%: B-40%; Rf=0.8; run time=7.5 min). Yield: 13.9 mg (26.2%). Yellow powder. LCMS purity: 100% (LCMS, Rf=0.57, run time=2 min). EI MS m/z: pos. 442.1 (MH+).

(4-methoxyphenyl)methyl N-{[2-(2,6-dioxopiperidin-3-yl)-3-oxo-2,3-dihydro-1H-isoindol-5-yl]methyl}carbamate (Compound 25) was obtained by Synthetic procedure 2 with using 70 mg (0.226 mmol) of 3-[6-(aminomethyl)-1-oxo-2,3-dihydro-1H-isoindol-2-yl]piperidine-2,6-dione hydrochloride (Reagent 1), 63 mg (0.456 mmol) of (4-methoxyphenyl)methanol (Reagent 2), 92 mg (0.567 mmol) of CDI, and 64.8 mg (0.501 mmol) of DIPEA. Purified by HPLC (gradient: from A-80%: B-20% to A-55%: B-45%; Rf=0.67; run time=6.5 min). Yield: 9 mg (9%). White powder. LCMS purity: 100% (LCMS, Rf=0.54, run time=2 min). EI MS m/z: pos. 438.2 (MH+).

(4-nitrophenyl)methyl N-{[2-(2,6-dioxopiperidin-3-yl)-3-oxo-2,3-dihydro-1H-isoindol-5-yl]methyl}carbamate (Compound 26) was obtained by Synthetic procedure 1 with using 34 mg (0.11 mmol) of 3-[6-(aminomethyl)-1-oxo-2,3-dihydro-1H-isoindol-2-yl]piperidine-2,6-dione hydrochloride (Reagent 1), 30 mg (0.139 mmol) of (4-nitrophenyl)methyl carbonochloridate (Reagent 2), and 48.4 mg (0.374 mmol) of DIPEA. Purified by HPLC (gradient: from A-85%: B-15% to A-65%: B-35%; Rf=0.84; run time=6.5 min). Yield: 7.7 mg (15.9%). Yellow powder. LCMS purity: 100% (LCMS, Rf=0.58, run time=2 min). EI MS m/z: pos. 453.2 (MH+).

{[1,1′-biphenyl]-4-yl}methyl N-{[2-(2,6-dioxopiperidin-3-yl)-3-oxo-2,3-dihydro-1H-isoindol-5-yl]methyl}carbamate (Compound 27) was obtained by Synthetic procedure 2 with using 46 mg (0.149 mmol) of 3-[6-(aminomethyl)-1-oxo-2,3-dihydro-1H-isoindol-2-yl]piperidine-2,6-dione hydrochloride (Reagent 1), 36 mg (0.195 mmol) of {[1,1′-biphenyl]-4-yl}methanol (Reagent 2), 44 mg (0.271 mmol) of CDI, and 42.7 mg (0.33 mmol) of DIPEA. Purified by HPLC (gradient: from A-60%: B-40% to A-40%: B-60%; Rf=0.54; run time=6.5 min). Yield: 10.5 mg (14.4%). White powder. LCMS purity: 98.5% (LCMS, Rf=0.68, run time=2 min). EI MS m/z: pos. 484.2 (MH+).

(6-methoxynaphthalen-2-yl)methyl N-{[2-(2,6-dioxopiperidin-3-yl)-3-oxo-2,3-dihydro-1H-isoindol-5-yl]methyl}carbamate (Compound 28) was obtained by Synthetic procedure 2 with using 64 mg (0.207 mmol) of 3-[6-(aminomethyl)-1-oxo-2,3-dihydro-1H-isoindol-2-yl]piperidine-2,6-dione hydrochloride (Reagent 1), 58 mg (0.308 mmol) of (6-methoxynaphthalen-2-yl)methanol (Reagent 2), 67 mg (0.413 mmol) of CDI, and 58.4 mg (0.452 mmol) of DIPEA. Purified by HPLC (gradient: from A-75%: B-25% to A-50%: B-50%; Rf=0.62; run time=6.5 min). Yield: 6.1 mg (6.1%). Yellow powder. LCMS purity: 100% (LCMS, Rf=0.54, run time=2 min). EI MS m/z: pos. 488.2 (MH+).

(4-methylphenyl)methyl N-{[2-(2,6-dioxopiperidin-3-yl)-3-oxo-2,3-dihydro-1H-isoindol-5-yl]methyl}carbamate (Compound 29) was obtained by Synthetic procedure 2 with using 50 mg (0.161 mmol) of 3-[6-(aminomethyl)-1-oxo-2,3-dihydro-1H-isoindol-2-yl]piperidine-2,6-dione hydrochloride (Reagent 1), 26 mg (0.213 mmol) of (4-methylphenyl)methanol (Reagent 2), 48 mg (0.296 mmol) of CDI, and 46.5 mg (0.36 mmol) of DIPEA. Purified by HPLC (gradient: from A-70%: B-30% to A-55%: B-45%; Rf=0.81; run time=6.5 min). Yield: 10.8 mg (15.7%). White powder. LCMS purity: 100% (LCMS, Rf=0.51, run time=2 min). EI MS m/z: pos. 422.0 (MH+).

(2,4-dimethylphenyl)methyl N-{[2-(2,6-dioxopiperidin-3-yl)-3-oxo-2,3-dihydro-1H-isoindol-5-yl]methyl}carbamate (Compound 30) was obtained by Synthetic procedure 2 with using 51 mg (0.165 mmol) of 3-[6-(aminomethyl)-1-oxo-2,3-dihydro-1H-isoindol-2-yl]piperidine-2,6-dione hydrochloride (Reagent 1), 30 mg (0.22 mmol) of (2,4-dimethylphenyl)methanol (Reagent 2), 49 mg (0.302 mmol) of CDI, and 48.2 mg (0.373 mmol) of DIPEA. Purified by HPLC (gradient: from A-75%: B-25% to A-50%: B-50%; Rf=0.74; run time=6.5 min). Yield: 11.5 mg (15.6%). Beige powder. LCMS purity: 100% (LCMS, Rf=0.53, run time=2 min). EI MS m/z: pos. 436.0 (MH+).

[3-(trifluoromethyl)phenyl]methyl N-{[2-(2,6-dioxopiperidin-3-yl)-3-oxo-2,3-dihydro-1H-isoindol-5-yl]methyl}carbamate (Compound 31) was obtained by Synthetic procedure 2 with using 46 mg (0.149 mmol) of 3-[6-(aminomethyl)-1-oxo-2,3-dihydro-1H-isoindol-2-yl]piperidine-2,6-dione hydrochloride (Reagent 1), 36 mg (0.204 mmol) of [3-(trifluoromethyl)phenyl]methanol (Reagent 2), 43 mg (0.265 mmol) of CDI, and 44.7 mg (0.346 mmol) of DIPEA. Purified by HPLC (gradient: from A-75%: B-25% to A-50%: B-50%; Rf=0.72; run time=6.5 min). Yield: 5.3 mg (7.1%). Yellow powder. LCMS purity: 100% (LCMS, Rf=0.53, run time=2 min). EI MS m/z: pos. 476.0 (MH+).

(3-nitrophenyl)methyl N-{[2-(2,6-dioxopiperidin-3-yl)-3-oxo-2,3-dihydro-1H-isoindol-5-yl]methyl}carbamate (Compound 32) was obtained by Synthetic procedure 2 with using 48 mg (0.155 mmol) of 3-[6-(aminomethyl)-1-oxo-2,3-dihydro-1H-isoindol-2-yl]piperidine-2,6-dione hydrochloride (Reagent 1), 31 mg (0.202 mmol) of (3-nitrophenyl)methanol (Reagent 2), 43 mg (0.265 mmol) of CDI, and 44.3 mg (0.343 mmol) of DIPEA. Purified by HPLC (gradient: from A-90%: B-10% to A-65%: B-35%; Rf=0.92; run time=6.7 min). Yield: 21.5 mg (30.5%). White powder. LCMS purity: 95.2% (LCMS, Rf=0.58, run time=2 min). EI MS m/z: pos. 453.2 (MH+).

[4-(difluoromethoxy)phenyl]methyl N-{[2-(2,6-dioxopiperidin-3-yl)-3-oxo-2,3-dihydro-1H-isoindol-5-yl]methyl}carbamate (Compound 33) was obtained by Synthetic procedure 2 with using 44 mg (0.142 mmol) of 3-[6-(aminomethyl)-1-oxo-2,3-dihydro-1H-isoindol-2-yl]piperidine-2,6-dione hydrochloride (Reagent 1), 32 mg (0.184 mmol) of [4-(difluoromethoxy)phenyl]methanol (Reagent 2), 46 mg (0.284 mmol) of CDI, and 40.2 mg (0.311 mmol) of DIPEA. Purified by HPLC (gradient: from A-70%: B-30% to A-55%: B-45%; Rf=0.61; run time=6.5 min). Yield: 5.8 mg (8.7%). White powder. LCMS purity: 99% (LCMS, Rf=0.57, run time=2 min). EI MS m/z: pos. 474.0 (MH+).

(4-ethoxy-3,5-dimethylphenyl)methyl N-{[2-(2,6-dioxopiperidin-3-yl)-3-oxo-2,3-dihydro-1H-isoindol-5-yl]methyl}carbamate (Compound 34) was obtained by Synthetic procedure 2 with using 41 mg (0.132 mmol) of 3-[6-(aminomethyl)-1-oxo-2,3-dihydro-1H-isoindol-2-yl]piperidine-2,6-dione hydrochloride (Reagent 1), 32 mg (0.178 mmol) of (4-ethoxy-3,5-dimethylphenyl)methanol (Reagent 2), 43 mg (0.265 mmol) of CDI, and 38.8 mg (0.3 mmol) of DIPEA. Purified by HPLC (gradient: from A-70%: B-30% to A-45%: B-55%; Rf=0.69; run time=6.5 min). Yield: 16.6 mg (25.3%). White powder. LCMS purity: 96.8% (LCMS, Rf=0.63, run time=2 min). EI MS m/z: pos. 480.2 (MH+).

[3-(methylsulfanyl)phenyl]methyl N-{[2-(2,6-dioxopiperidin-3-yl)-3-oxo-2,3-dihydro-1H-isoindol-5-yl]methyl}carbamate (Compound 35) was obtained by Synthetic procedure 2 with using 49 mg (0.158 mmol) of 3-[6-(aminomethyl)-1-oxo-2,3-dihydro-1H-isoindol-2-yl]piperidine-2,6-dione hydrochloride (Reagent 1), 30 mg (0.195 mmol) of [3-(methylsulfanyl)phenyl]methanol (Reagent 2), 45 mg (0.278 mmol) of CDI, and 42.5 mg (0.329 mmol) of DIPEA. Purified by HPLC (gradient: from A-70%: B-30% to A-55%: B-45%; Rf=0.64; run time=6.5 min). Yield: 15.1 mg (22.3%). Beige powder. LCMS purity: 100% (LCMS, Rf=0.51, run time=2 min). EI MS m/z: pos. 454.0 (MH+).

[4-(2-methylbutan-2-yl)phenyl]methyl N-{[2-(2,6-dioxopiperidin-3-yl)-3-oxo-2,3-dihydro-1H-isoindol-5-yl]methyl}carbamate (Compound 36) was obtained by Synthetic procedure 2 with using 44 mg (0.142 mmol) of 3-[6-(aminomethyl)-1-oxo-2,3-dihydro-1H-isoindol-2-yl]piperidine-2,6-dione hydrochloride (Reagent 1), 33 mg (0.185 mmol) of [4-(2-methylbutan-2-yl)phenyl]methanol (Reagent 2), 47 mg (0.29 mmol) of CDI, and 40.5 mg (0.313 mmol) of DIPEA. Purified by HPLC (gradient: from A-55%: B-45% to A-40%: B-60%; Rf=0.75; run time=6.5 min). Yield: 23.6 mg (34.7%). White powder. LCMS purity: 96.5% (LCMS, Rf=0.72, run time=2 min). EI MS m/z: pos. 478.2 (MH+).

(4-cyclopentylphenyl)methyl N-{[2-(2,6-dioxopiperidin-3-yl)-3-oxo-2,3-dihydro-1H-isoindol-5-yl]methyl}carbamate (Compound 37) was obtained by Synthetic procedure 2 with using 43 mg (0.139 mmol) of 3-[6-(aminomethyl)-1-oxo-2,3-dihydro-1H-isoindol-2-yl]piperidine-2,6-dione hydrochloride (Reagent 1), 32 mg (0.182 mmol) of (4-cyclopentylphenyl)methanol (Reagent 2), 42 mg (0.259 mmol) of CDI, and 39.7 mg (0.307 mmol) of DIPEA. Purified by HPLC (gradient: from A-55%: B-45% to A-40%: B-60%; Rf=0.71; run time=6.5 min). Yield: 26.3 mg (39.6%). White powder. LCMS purity: 100% (LCMS, Rf=0.7, run time=2 min). EI MS m/z: pos. 476.2 (MH+).

[3-(propan-2-yloxy)phenyl]methyl N-{[2-(2,6-dioxopiperidin-3-yl)-3-oxo-2,3-dihydro-1H-isoindol-5-yl]methyl}carbamate (Compound 38) was obtained by Synthetic procedure 2 with using 54 mg (0.174 mmol) of 3-[6-(aminomethyl)-1-oxo-2,3-dihydro-1H-isoindol-2-yl]piperidine-2,6-dione hydrochloride (Reagent 1), 35 mg (0.211 mmol) of [3-(propan-2-yloxy)phenyl]methanol (Reagent 2), 50 mg (0.308 mmol) of CDI, and 46.1 mg (0.357 mmol) of DIPEA. Purified by HPLC (gradient: from A-65%: B-35% to A-50%: B-50%; Rf=0.59; run time=6.5 min). Yield: 10.5 mg (13.9%). Beige powder. LCMS purity: 100% (LCMS, Rf=0.53, run time=2 min). EI MS m/z: pos. 466.0 (MH+).

[4-(prop-2-yn-1-yloxy)phenyl]methyl N-{[2-(2,6-dioxopiperidin-3-yl)-3-oxo-2,3-dihydro-1H-isoindol-5-yl]methyl}carbamate (Compound 39) was obtained by Synthetic procedure 2 with using 48 mg (0.155 mmol) of 3-[6-(aminomethyl)-1-oxo-2,3-dihydro-1H-isoindol-2-yl]piperidine-2,6-dione hydrochloride (Reagent 1), 34 mg (0.21 mmol) of [4-(prop-2-yn-1-yloxy)phenyl]methanol (Reagent 2), 49 mg (0.302 mmol) of CDI, and 45.9 mg (0.355 mmol) of DIPEA. Purified by HPLC (gradient: from A-70%: B-30% to A-55%: B-45%; Rf=0.69; run time=6.5 min). Yield: 8.4 mg (11.3%). White powder. LCMS purity: 96.8% (LCMS, Rf=0.5, run time=2 min). EI MS m/z: pos. 462.0 (MH+).

[4-(cyclopropylmethoxy)phenyl]methyl N-{[2-(2,6-dioxopiperidin-3-yl)-3-oxo-2,3-dihydro-1H-isoindol-5-yl]methyl}carbamate (Compound 40) was obtained by Synthetic procedure 2 with using 64 mg (0.207 mmol) of 3-[6-(aminomethyl)-1-oxo-2,3-dihydro-1H-isoindol-2-yl]piperidine-2,6-dione hydrochloride (Reagent 1), 56 mg (0.314 mmol) of [4-(cyclopropylmethoxy)phenyl]methanol (Reagent 2), 68 mg (0.419 mmol) of CDI, and 59.6 mg (0.461 mmol) of DIPEA. Purified by HPLC (gradient: from A-75%: B-25% to A-50%: B-50%; Rf=0.92; run time=6.5 min). Yield: 5.9 mg (5.9%). Beige powder. LCMS purity: 100% (LCMS, Rf=0.56, run time=2 min). EI MS m/z: pos. 478.2 (MH+).

(4-ethoxy-2-methylphenyl)methyl N-{[2-(2,6-dioxopiperidin-3-yl)-3-oxo-2,3-dihydro-1H-isoindol-5-yl]methyl}carbamate (Compound 41) was obtained by Synthetic procedure 2 with using 53 mg (0.171 mmol) of 3-[6-(aminomethyl)-1-oxo-2,3-dihydro-1H-isoindol-2-yl]piperidine-2,6-dione hydrochloride (Reagent 1), 36 mg (0.217 mmol) of (4-ethoxy-2-methylphenyl)methanol (Reagent 2), 50 mg (0.308 mmol) of CDI, and 47.4 mg (0.367 mmol) of DIPEA. Purified by HPLC (gradient: from A-75%: B-25% to A-50%: B-50%; Rf=0.15; run time=6.5 min). Yield: 17 mg (21.9%). White powder. LCMS purity: 97.8% (LCMS, Rf=0.61, run time=2 min). EI MS m/z: pos. 466.2 (MH+).

[4-(2,2,2-trifluoroethyl)phenyl]methyl N-{[2-(2,6-dioxopiperidin-3-yl)-3-oxo-2,3-dihydro-1H-isoindol-5-yl]methyl}carbamate (Compound 42) was obtained by Synthetic procedure 2 with using 49 mg (0.158 mmol) of 3-[6-(aminomethyl)-1-oxo-2,3-dihydro-1H-isoindol-2-yl]piperidine-2,6-dione hydrochloride (Reagent 1), 38 mg (0.2 mmol) of [4-(2,2,2-trifluoroethyl)phenyl]methanol (Reagent 2), 46 mg (0.284 mmol) of CDI, and 43.7 mg (0.338 mmol) of DIPEA. Purified by HPLC (gradient: from A-70%: B-30% to A-45%: B-55%; Rf=0.67; run time=6.5 min). Yield: 14.3 mg (19%). White powder. LCMS purity: 100% (LCMS, Rf=0.6, run time=2 min). EI MS m/z: pos. 490.0 (MH+).

(4-propylphenyl)methyl N-{[2-(2,6-dioxopiperidin-3-yl)-3-oxo-2,3-dihydro-1H-isoindol-5-yl]methyl}carbamate (Compound 43) was obtained by Synthetic procedure 2 with using 45 mg (0.145 mmol) of 3-[6-(aminomethyl)-1-oxo-2,3-dihydro-1H-isoindol-2-yl]piperidine-2,6-dione hydrochloride (Reagent 1), 29 mg (0.193 mmol) of (4-propylphenyl)methanol (Reagent 2), 43 mg (0.265 mmol) of CDI, and 42.2 mg (0.327 mmol) of DIPEA. Purified by HPLC (gradient: from A-70%: B-30% to A-45%: B-55%; Rf=0.79; run time=6.5 min). Yield: 21.7 mg (32.5%). Yellow powder. LCMS purity: 100% (LCMS, Rf=0.66, run time=2 min). EI MS m/z: pos. 450.2 (MH+).

(4-fluoro-3-methoxyphenyl)methyl N-{[2-(2,6-dioxopiperidin-3-yl)-3-oxo-2,3-dihydro-1H-isoindol-5-yl]methyl}carbamate (Compound 44) was obtained by Synthetic procedure 2 with using 55 mg (0.178 mmol) of 3-[6-(aminomethyl)-1-oxo-2,3-dihydro-1H-isoindol-2-yl]piperidine-2,6-dione hydrochloride (Reagent 1), 33 mg (0.211 mmol) of (4-fluoro-3-methoxyphenyl)methanol (Reagent 2), 48 mg (0.296 mmol) of CDI, and 46.2 mg (0.357 mmol) of DIPEA. Purified by HPLC (gradient: from A-70%: B-30% to A-60%: B-40%; Rf=0.56; run time=6.5 min). Yield: 40.6 mg (54.8%). White powder. LCMS purity: 91.3% (LCMS, Rf=0.56, run time=2 min). EI MS m/z: pos. 456.2 (MH+).

(3-tert-butylphenyl)methyl N-{[2-(2,6-dioxopiperidin-3-yl)-3-oxo-2,3-dihydro-1H-isoindol-5-yl]methyl}carbamate (Compound 45) was obtained by Synthetic procedure 2 with using 41 mg (0.132 mmol) of 3-[6-(aminomethyl)-1-oxo-2,3-dihydro-1H-isoindol-2-yl]piperidine-2,6-dione hydrochloride (Reagent 1), 29 mg (0.177 mmol) of (3-tert-butylphenyl)methanol (Reagent 2), 42 mg (0.259 mmol) of CDI, and 38.6 mg (0.299 mmol) of DIPEA. Purified by HPLC (gradient: from A-55%: B-45% to A-40%: B-60%; Rf=0.55; run time=6.5 min). Yield: 13.9 mg (22.1%). Beige powder. LCMS purity: 100% (LCMS, Rf=0.57, run time=2 min). EI MS m/z: pos. 464.0 (MH+).

[4-(2-methylpropoxy)phenyl]methyl N-{[2-(2,6-dioxopiperidin-3-yl)-3-oxo-2,3-dihydro-1H-isoindol-5-yl]methyl}carbamate (Compound 46) was obtained by Synthetic procedure 2 with using 45 mg (0.145 mmol) of 3-[6-(aminomethyl)-1-oxo-2,3-dihydro-1H-isoindol-2-yl]piperidine-2,6-dione hydrochloride (Reagent 1), 34 mg (0.189 mmol) of [4-(2-methylpropoxy)phenyl]methanol (Reagent 2), 46 mg (0.284 mmol) of CDI, and 41.3 mg (0.32 mmol) of DIPEA. Purified by HPLC (gradient: from A-70%: B-30% to A-45%: B-55%; Rf=0.75; run time=6.5 min). Yield: 7.8 mg (11.2%). White powder. LCMS purity: 97% (LCMS, Rf=0.58, run time=2 min). EI MS m/z: pos. 480.0 (MH+).

[4-methoxy-3-(propan-2-yl)phenyl]methyl N-{[2-(2,6-dioxopiperidin-3-yl)-3-oxo-2,3-dihydro-1H-isoindol-5-yl]methyl}carbamate (Compound 47) was obtained by Synthetic procedure 2 with using 63 mg (0.203 mmol) of 3-[6-(aminomethyl)-1-oxo-2,3-dihydro-1H-isoindol-2-yl]piperidine-2,6-dione hydrochloride (Reagent 1), 56 mg (0.311 mmol) of [4-methoxy-3-(propan-2-yl)phenyl]methanol (Reagent 2), 68 mg (0.419 mmol) of CDI, and 58.9 mg (0.456 mmol) of DIPEA. Purified by HPLC (gradient: from A-75%: B-25% to A-50%: B-50%; Rf=0.57; run time=6.5 min). Yield: 6.2 mg (6.2%). Yellow powder. LCMS purity: 100% (LCMS, Rf=0.64, run time=2 min). EI MS m/z: pos. 163.2, 497.2, 598.2, 389.2, 325.2.

[4-(3-fluorooxetan-3-yl)phenyl]methyl N-{[2-(2,6-dioxopiperidin-3-yl)-3-oxo-2,3-dihydro-1H-isoindol-5-yl]methyl}carbamate (Compound 48) was obtained by Synthetic procedure 2 with using 43 mg (0.139 mmol) of 3-[6-(aminomethyl)-1-oxo-2,3-dihydro-1H-isoindol-2-yl]piperidine-2,6-dione hydrochloride (Reagent 1), 33 mg (0.181 mmol) of [4-(3-fluorooxetan-3-yl)phenyl]methanol (Reagent 2), 43 mg (0.265 mmol) of CDI, and 39.6 mg (0.306 mmol) of DIPEA. Purified by HPLC (gradient: from A-75%: B-25% to A-65%: B-35%; Rf=0.55; run time=7 min). Yield: 7.1 mg (10.6%). Beige powder. LCMS purity: 99% (LCMS, Rf=0.53, run time=2 min). EI MS m/z: pos. 462.2.

[3-chloro-4-(methylsulfanyl)phenyl]methyl N-{[2-(2,6-dioxopiperidin-3-yl)-3-oxo-2,3-dihydro-1H-isoindol-5-yl]methyl}carbamate (Compound 49) was obtained by Synthetic procedure 2 with using 48 mg (0.155 mmol) of 3-[6-(aminomethyl)-1-oxo-2,3-dihydro-1H-isoindol-2-yl]piperidine-2,6-dione hydrochloride (Reagent 1), 34 mg (0.18 mmol) of [3-chloro-4-(methylsulfanyl)phenyl]methanol (Reagent 2), 46 mg (0.284 mmol) of CDI, and 39.4 mg (0.305 mmol) of DIPEA. Purified by HPLC (gradient: from A-70%: B-30% to A-45%: B-55%; Rf=0.15; run time=6.5 min). Yield: 14.5 mg (21.4%). White powder. LCMS purity: 96.6% (LCMS, Rf=0.62, run time=2 min). EI MS m/z: pos. 488.2 (MH+).

[4-(3,3,3-trifluoroprop-1-yn-1-yl)phenyl]methyl N-{[2-(2,6-dioxopiperidin-3-yl)-3-oxo-2,3-dihydro-1H-isoindol-5-yl]methyl}carbamate (Compound 50) was obtained by Synthetic procedure 2 with using 41 mg (0.132 mmol) of 3-[6-(aminomethyl)-1-oxo-2,3-dihydro-1H-isoindol-2-yl]piperidine-2,6-dione hydrochloride (Reagent 1), 34 mg (0.17 mmol) of [4-(3,3,3-trifluoroprop-1-yn-1-yl)phenyl]methanol (Reagent 2), 37 mg (0.228 mmol) of CDI, and 37.2 mg (0.288 mmol) of DIPEA. Purified by HPLC (gradient: from A-65%: B-35% to A-50%: B-50%; Rf=0.83; run time=6.5 min). Yield: 5.6 mg (8.6%). Beige powder. LCMS purity: 99% (LCMS, Rf=0.67, run time=2 min). EI MS m/z: pos. 499.9 (MH+).

{4′-fluoro-[1,1′-biphenyl]-3-yl}methyl N-{[2-(2,6-dioxopiperidin-3-yl)-3-oxo-2,3-dihydro-1H-isoindol-5-yl]methyl}carbamate (Compound 51) was obtained by Synthetic procedure 2 with using 50 mg (0.161 mmol) of 3-[6-(aminomethyl)-1-oxo-2,3-dihydro-1H-isoindol-2-yl]piperidine-2,6-dione hydrochloride (Reagent 1), 43 mg (0.213 mmol) of {4′-fluoro-[1,1′-biphenyl]-3-yl}methanol (Reagent 2), 51 mg (0.315 mmol) of CDI, and 46.5 mg (0.36 mmol) of DIPEA. Purified by HPLC (gradient: from A-65%: B-35% to A-40%: B-60%; Rf=0.7; run time=6.5 min). Yield: 20.2 mg (24.6%). White powder. LCMS purity: 91.2% (LCMS, Rf=0.58, run time=2 min). EI MS m/z: pos. 502.0 (MH+).

[4-(4-methyl-1,3-thiazol-5-yl)phenyl]methyl N-{[2-(2,6-dioxopiperidin-3-yl)-3-oxo-2,3-dihydro-1H-isoindol-5-yl]methyl}carbamate (Compound 52) was obtained by Synthetic procedure 2 with using 45 mg (0.145 mmol) of 3-[6-(aminomethyl)-1-oxo-2,3-dihydro-1H-isoindol-2-yl]piperidine-2,6-dione hydrochloride (Reagent 1), 37 mg (0.18 mmol) of [4-(4-methyl-1,3-thiazol-5-yl)phenyl]methanol (Reagent 2), 43 mg (0.265 mmol) of CDI, and 39.4 mg (0.305 mmol) of DIPEA. Purified by HPLC (gradient: from A-70%: B-30% to A-55%: B-45%; Rf=0.54; run time=6.5 min). Yield: 3.8 mg (5.4%). White powder. LCMS purity: 100% (LCMS, Rf=0.56, run time=2 min). EI MS m/z: pos. 505.0 (MH+).

{4-[(piperidin-1-yl)methyl]phenyl}methyl N-{[2-(2,6-dioxopiperidin-3-yl)-3-oxo-2,3-dihydro-1H-isoindol-5-yl]methyl}carbamate (Compound 53) was obtained by Synthetic procedure 2 with using 48 mg (0.155 mmol) of 3-[6-(aminomethyl)-1-oxo-2,3-dihydro-1H-isoindol-2-yl]piperidine-2,6-dione hydrochloride (Reagent 1), 46 mg (0.19 mmol) of {4-[(piperidin-1-yl)methyl]phenyl}methanol hydrochloride (Reagent 2), 42 mg (0.259 mmol) of CDI, 41.6 mg (0.322 mmol) of DIPEA, and 26.5 mg (0.205 mmol) of DIPEA. Purified by HPLC (gradient: from A-75%: B-25% to A-50%: B-50%; Rf=0.97; run time=5.6 min). Yield: 10.5 mg (14.2%). Brown powder. LCMS purity: 98% (LCMS, Rf=0.37, run time=2 min). EI MS m/z: pos. 505.0 (MH+).

[3-(2,2,2-trifluoroethoxy)phenyl]methyl N-{[2-(2,6-dioxopiperidin-3-yl)-3-oxo-2,3-dihydro-1H-isoindol-5-yl]methyl}carbamate (Compound 54) was obtained by Synthetic procedure 2 with using 52 mg (0.168 mmol) of 3-[6-(aminomethyl)-1-oxo-2,3-dihydro-1H-isoindol-2-yl]piperidine-2,6-dione hydrochloride (Reagent 1), 43 mg (0.209 mmol) of [3-(2,2,2-trifluoroethoxy)phenyl]methanol (Reagent 2), 50 mg (0.308 mmol) of CDI, and 45.6 mg (0.353 mmol) of DIPEA. Purified by HPLC (gradient: from A-70%: B-30% to A-45%: B-55%; Rf=0.68; run time=6.5 min). Yield: 8.8 mg (10.9%). Beige powder. LCMS purity: 100% (LCMS, Rf=0.61, run time=2 min). EI MS m/z: pos. 505.9 (MH+).

{4-[(morpholin-4-yl)methyl]phenyl}methyl N-{[2-(2,6-dioxopiperidin-3-yl)-3-oxo-2,3-dihydro-1H-isoindol-5-yl]methyl}carbamate (Compound 55) was obtained by Synthetic procedure 2 with using 46 mg (0.149 mmol) of 3-[6-(aminomethyl)-1-oxo-2,3-dihydro-1H-isoindol-2-yl]piperidine-2,6-dione hydrochloride (Reagent 1), 41 mg (0.198 mmol) of {4-[(morpholin-4-yl)methyl]phenyl}methanol (Reagent 2), 46 mg (0.284 mmol) of CDI, and 43.3 mg (0.335 mmol) of DIPEA. Purified by HPLC (gradient: from A-95%: B-5% to A-80%: B-20%; Rf=0.51; run time=6.5 min). Yield: 8.7 mg (11.3%). Yellow powder. LCMS purity: 100% (LCMS, Rf=0.39, run time=2 min). EI MS m/z: pos. 507.2 (MH+).

{3-[(trifluoromethyl)sulfanyl]phenyl}methyl N-{[2-(2,6-dioxopiperidin-3-yl)-3-oxo-2,3-dihydro-1H-isoindol-5-yl]methyl}carbamate (Compound 56) was obtained by Synthetic procedure 2 with using 38 mg (0.123 mmol) of 3-[6-(aminomethyl)-1-oxo-2,3-dihydro-1H-isoindol-2-yl]piperidine-2,6-dione hydrochloride (Reagent 1), 36 mg (0.173 mmol) of {3-[(trifluoromethyl)sulfanyl]phenyl}methanol (Reagent 2), 42 mg (0.259 mmol) of CDI, and 37.8 mg (0.292 mmol) of DIPEA. Purified by HPLC (gradient: from A-65%: B-35% to A-40%: B-60%; Rf=0.64; run time=6.5 min). Yield: 35.5 mg (52.6%). White powder. LCMS purity: 100% (LCMS, Rf=0.65, run time=2 min). EI MS m/z: pos. 508.0 (MH+).

[2-fluoro-4-(trifluoromethoxy)phenyl]methyl N-{[2-(2,6-dioxopiperidin-3-yl)-3-oxo-2,3-dihydro-1H-isoindol-5-yl]methyl}carbamate (Compound 57) was obtained by Synthetic procedure 2 with using 47 mg (0.152 mmol) of 3-[6-(aminomethyl)-1-oxo-2,3-dihydro-1H-isoindol-2-yl]piperidine-2,6-dione hydrochloride (Reagent 1), 42 mg (0.2 mmol) of [2-fluoro-4-(trifluoromethoxy)phenyl]methanol (Reagent 2), 45 mg (0.278 mmol) of CDI, and 43.7 mg (0.338 mmol) of DIPEA. Purified by HPLC (gradient: from A-70%: B-30% to A-45%: B-55%; Rf=0.7; run time=6.5 min). Yield: 28.8 mg (36.8%). White powder. LCMS purity: 100% (LCMS, Rf=0.55, run time=2 min). EI MS m/z: pos. 510.0 (MH+).

{4′-methoxy-[1,1′-biphenyl]-3-yl}methyl N-{[2-(2,6-dioxopiperidin-3-yl)-3-oxo-2,3-dihydro-1H-isoindol-5-yl]methyl}carbamate (Compound 58) was obtained by Synthetic procedure 2 with using 44 mg (0.142 mmol) of 3-[6-(aminomethyl)-1-oxo-2,3-dihydro-1H-isoindol-2-yl]piperidine-2,6-dione hydrochloride (Reagent 1), 39 mg (0.182 mmol) of {4′-methoxy-[1,1′-biphenyl]-3-yl}methanol (Reagent 2), 43 mg (0.265 mmol) of CDI, and 39.8 mg (0.308 mmol) of DIPEA. Purified by HPLC (gradient: from A-55%: B-45% to A-40%: B-60%; Rf=0.58; run time=6.5 min). Yield: 20.1 mg (28%). White powder. LCMS purity: 100% (LCMS, Rf=0.65, run time=2 min). EI MS m/z: pos. 514.2 (MH+).

[4-(trifluoromethyl)phenyl]methyl N-{[2-(2,6-dioxopiperidin-3-yl)-3-oxo-2,3-dihydro-1H-isoindol-5-yl]methyl}carbamate (Compound 59) was obtained by Synthetic procedure 2 with using 47 mg (0.152 mmol) of 3-[6-(aminomethyl)-1-oxo-2,3-dihydro-1H-isoindol-2-yl]piperidine-2,6-dione hydrochloride (Reagent 1), 33 mg (0.187 mmol) of [4-(trifluoromethyl)phenyl]methanol (Reagent 2), 46 mg (0.284 mmol) of CDI, and 41 mg (0.317 mmol) of DIPEA. Purified by HPLC (gradient: from A-75%: B-25% to A-50%: B-50%; Rf=0.74; run time=6.5 min). Yield: 10.1 mg (14.7%). White powder. LCMS purity: 100% (LCMS, Rf=0.45, run time=6 min). EI MS m/z: pos. 476.0 (MH+).

[4-(propan-2-yl)phenyl]methyl N-{[2-(2,6-dioxopiperidin-3-yl)-3-oxo-2,3-dihydro-1H-isoindol-5-yl]methyl}carbamate (Compound 60) was obtained by Synthetic procedure 2 with using 68.9 mg (0.222 mmol) of 3-[6-(aminomethyl)-1-oxo-2,3-dihydro-1H-isoindol-2-yl]piperidine-2,6-dione hydrochloride (Reagent 1), 50.1 mg (0.334 mmol) of [4-(propan-2-yl)phenyl]methanol (Reagent 2), 72.1 mg (0.445 mmol) of CDI, and 63.3 mg (0.49 mmol) of DIPEA. Purified by HPLC (gradient: from A-80%: B-20% to A-55%: B-45%; Rf=0.85; run time=6.5 min). Yield: 4.8 mg (4.8%). Brown powder. LCMS purity: 98.4% (LCMS, Rf=0.64, run time=2 min). EI MS m/z: pos. 450.0 (MH+).

[6-(2,2,2-trifluoroethoxy)pyridin-3-yl]methyl N-{[2-(2,6-dioxopiperidin-3-yl)-3-oxo-2,3-dihydro-1H-isoindol-5-yl]methyl}carbamate (Compound 61) was obtained by Synthetic procedure 2 with using 40 mg (0.129 mmol) of 3-[6-(aminomethyl)-1-oxo-2,3-dihydro-1H-isoindol-2-yl]piperidine-2,6-dione hydrochloride (Reagent 1), 36 mg (0.174 mmol) of [6-(2,2,2-trifluoroethoxy)pyridin-3-yl]methanol (Reagent 2), 38 mg (0.234 mmol) of CDI, and 38 mg (0.294 mmol) of DIPEA. Purified by HPLC (gradient: from A-75%: B-25% to A-55%: B-45%; Rf=0.74; run time=6.5 min). Yield: 19.2 mg (28.4%). Beige powder. LCMS purity: 100% (LCMS, Rf=0.64, run time=2 min). EI MS m/z: pos. 507.2 (MH+).

[4-(2,2,2-trifluoroethoxy)phenyl]methyl N-{[2-(2,6-dioxopiperidin-3-yl)-3-oxo-2,3-dihydro-1H-isoindol-5-yl]methyl}carbamate (Compound 62) was obtained by Synthetic procedure 2 with using 42 mg (0.136 mmol) of 3-[6-(aminomethyl)-1-oxo-2,3-dihydro-1H-isoindol-2-yl]piperidine-2,6-dione hydrochloride (Reagent 1), 35 mg (0.17 mmol) of [4-(2,2,2-trifluoroethoxy)phenyl]methanol (Reagent 2), 38 mg (0.234 mmol) of CDI, and 37.1 mg (0.287 mmol) of DIPEA. Purified by HPLC (gradient: from A-75%: B-25% to A-60%: B-40%; Rf=0.5; run time=8 min). Yield: 9.1 mg (13.8%). Beige powder. LCMS purity: 100% (LCMS, Rf=0.64, run time=2 min). EI MS m/z: pos. 445.2, 189.2, 377.2, 523.2, 462.2.

[4-(1,1,1-trifluoro-2-methylpropan-2-yl)phenyl]methyl N-{[2-(2,6-dioxopiperidin-3-yl)-3-oxo-2,3-dihydro-1H-isoindol-5-yl]methyl}carbamate (Compound 63) was obtained by Synthetic procedure 2 with using 37 mg (0.119 mmol) of 3-[6-(aminomethyl)-1-oxo-2,3-dihydro-1H-isoindol-2-yl]piperidine-2,6-dione hydrochloride (Reagent 1), 34 mg (0.156 mmol) of [4-(1,1,1-trifluoro-2-methylpropan-2-yl)phenyl]methanol (Reagent 2), 37 mg (0.228 mmol) of CDI, and 34.1 mg (0.264 mmol) of DIPEA. Purified by HPLC (gradient: from A-65%: B-35% to A-40%: B-60%; Rf=0.64; run time=6.5 min). Yield: 13.1 mg (21.1%). White powder. LCMS purity: 100% (LCMS, Rf=0.61, run time=2 min). EI MS m/z: pos. 518.4 (MH+).

(4-cyclopropylphenyl)methyl N-{[2-(2,6-dioxopiperidin-3-yl)-3-oxo-2,3-dihydro-1H-isoindol-5-yl]methyl}carbamate (Compound 64) was obtained by Synthetic procedure 2 with using 54 mg (0.174 mmol) of 3-[6-(aminomethyl)-1-oxo-2,3-dihydro-1H-isoindol-2-yl]piperidine-2,6-dione hydrochloride (Reagent 1), 33 mg (0.223 mmol) of (4-cyclopropylphenyl)methanol (Reagent 2), 53 mg (0.327 mmol) of CDI, and 48.7 mg (0.377 mmol) of DIPEA. Purified by HPLC (gradient: from A-75%: B-25% to A-50%: B-50%; Rf=0.72; run time=6.5 min). Yield: 14.4 mg (18.8%). White powder. LCMS purity: 100% (LCMS, Rf=0.6, run time=2 min). EI MS m/z: pos. 448.2 (MH+).

[6-(oxan-4-yloxy)pyridin-3-yl]methyl N-{[2-(2,6-dioxopiperidin-3-yl)-3-oxo-2,3-dihydro-1H-isoindol-5-yl]methyl}carbamate (Compound 65) was obtained by Synthetic procedure 2 with using 93 mg (0.3 mmol) of 3-[6-(aminomethyl)-1-oxo-2,3-dihydro-1H-isoindol-2-yl]piperidine-2,6-dione hydrochloride (Reagent 1), 81 mg (0.387 mmol) of [6-(oxan-4-yloxy)pyridin-3-yl]methanol (Reagent 2), 92 mg (0.567 mmol) of CDI, and 84.7 mg (0.655 mmol) of DIPEA. Purified by HPLC (gradient: from A-85%: B-15% to A-65%: B-35%; Rf=0.71; run time=6.5 min). Yield: 39.2 mg (25.9%). White powder. LCMS purity: 90.7% (LCMS, Rf=0.5, run time=2 min). EI MS m/z: pos. 509.2 (MH+).

[6-(trifluoromethyl)pyridin-3-yl]methyl N-{[2-(2,6-dioxopiperidin-3-yl)-3-oxo-2,3-dihydro-1H-isoindol-5-yl]methyl}carbamate (Compound 66) was obtained by Synthetic procedure 2 with using 52 mg (0.168 mmol) of 3-[6-(aminomethyl)-1-oxo-2,3-dihydro-1H-isoindol-2-yl]piperidine-2,6-dione hydrochloride (Reagent 1), 38 mg (0.215 mmol) of [6-(trifluoromethyl)pyridin-3-yl]methanol (Reagent 2), 47 mg (0.29 mmol) of CDI, and 46.9 mg (0.363 mmol) of DIPEA. Purified by HPLC (gradient: from A-75%: B-25% to A-55%: B-45%; Rf=0.54; run time=6.5 min). Yield: 7.6 mg (9.7%). White powder. LCMS purity: 100% (LCMS, Rf=0.58, run time=2 min). EI MS m/z: pos. 477.2 (MH+).

The following experimental procedures were used to prepare the compounds of Example 23 below.

### Synthetic Procedure 1

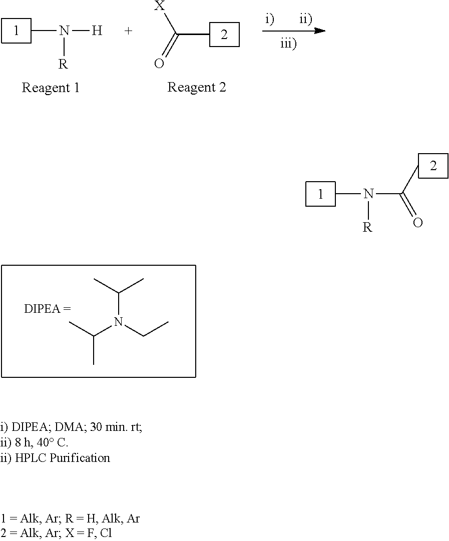

Reagent 1 (appr. 1 eq.), and Reagent 2 (appr. 1.3-2 eq.), and Diisopropylethylamine (DIPEA) (appr. 3.8-5 eq.) were mixed in dry Dimethylacetamide (DMA) (appr. 0.7 ml per 100 mg of product). The reaction mixture was sealed and heated for 8 hours at 40°, then cooled to the ambient temperature, and the solvent was evaporated under reduced pressure. The residue was dissolved in the DMSO (appr. 1 ml up to 300 mg of product) and DMSO solution was filtered, analyzed by LCMS, and transferred for HPLC purification.

### Synthetic Procedure 2

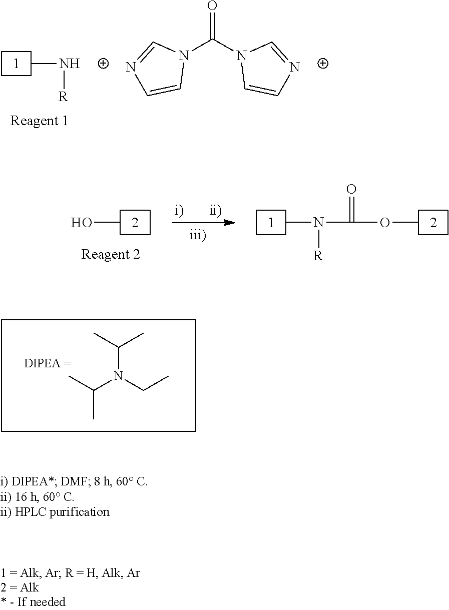

Reagent 2 (appr. 1.3-2 eq.) and 1,1′-Carbonyldiimidazole (CDI) (appr. 1.5-2.5 eq.) were mixed in dry Dimethylacetamide (DMA) (appr. 0.7 ml per 100 mg of product). The reaction mixture was sealed and heated for 8 hours at 60° C. Then the mixture was cooled to the ambient temperature and Reagent 1 (appr. 1 eq.) was added in one portion. The reaction mixture was sealed and heated for 16 hours at 60° C., and cooled to the ambient temperature. The solvent was evaporated under reduced pressure and the residue was dissolved in the DMSO (appr. 1 ml up to 300 mg of product). DMSO solution was filtered, analyzed by LCMS, and transferred for HPLC purification.

### Example 23. Syntheses of Compounds 67-68

2-phenylbutyl N-{[2-(2,6-dioxopiperidin-3-yl)-3-oxo-2,3-dihydro-1H-isoindol-5-yl]methyl}carbamate (Compound 67) was obtained by Synthetic procedure 2 with using 57 mg (0.184 mmol) of 3-[6-(aminomethyl)-1-oxo-2,3-dihydro-1H-isoindol-2-yl]piperidine-2,6-dione hydrochloride (Reagent 1), 37 mg (0.246 mmol) of 2-phenylbutan-1-ol (Reagent 2), 58 mg (0.358 mmol) of CDI, and 53.9 mg (0.417 mmol) of DIPEA. Purified by HPLC (gradient: from A-70%: B-30% to A-55%: B-45%; Rf=0.56; run time=8.2 min). Yield: 13.8 mg (16.2%). Yellow sticky oil. LCMS purity: 98.5% (LCMS, Rf=0.58, run time=2 min). EI MS m/z: pos. 450.2 (MH+).

(2R)-1-phenylbutan-2-yl N-{[2-(2,6-dioxopiperidin-3-yl)-3-oxo-2,3-dihydro-1H-isoindol-5-yl]methyl}carbamate (Compound 68) was obtained by Synthetic procedure 2 with using 63 mg (0.203 mmol) of 3-[6-(aminomethyl)-1-oxo-2,3-dihydro-1H-isoindol-2-yl]piperidine-2,6-dione hydrochloride (Reagent 1), 45 mg (0.3 mmol) of (2R)-1-phenylbutan-2-ol (Reagent 2), 49 mg (0.302 mmol) of CDI, and 56.8 mg (0.439 mmol) of DIPEA. Purified by HPLC (gradient: from A-70%: B-30% to A-55%: B-45%; Rf=0.57; run time=8.5 min). Yield: 4.7 mg (5.2%). Light brown sticky oil. LCMS purity: 96.6% (LCMS, Rf=0.46, run time=2.5 min). EI MS m/z: pos. 450.0 (MH+).

### Example 24. Syntheses of Compound 69

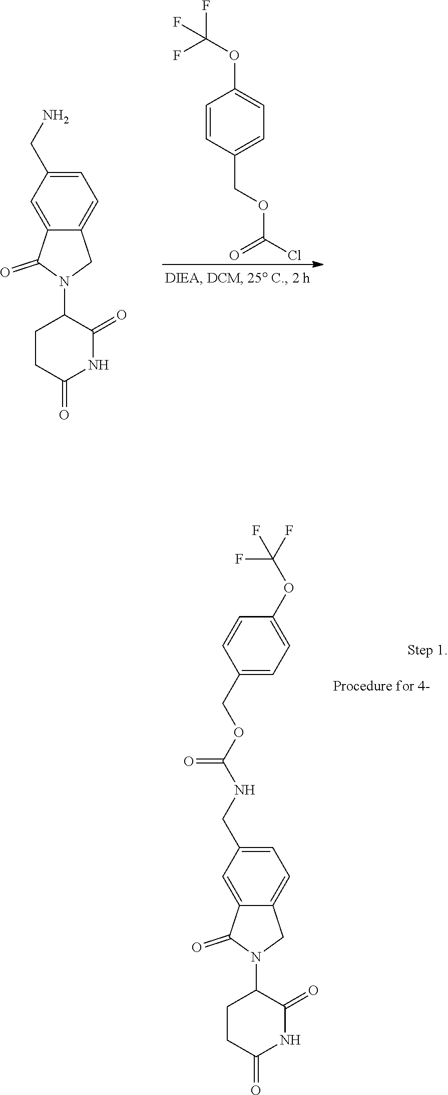 [SMILES: NCc1ccc2c(c1)C(=O)N(C1CCC(=O)NC1=O)C2 ; O=C(Cl)OCc1ccc(OC(F)(F)F)cc1 ; O=C1CCC(N2Cc3ccc(CNC(=O)OCc4ccc(OC(F)(F)F)cc4)cc3C2=O)C(=O)N1]

### (trifluoromethoxy)benzyl ((2-(2,6-dioxopiperidin-3-yl)-3-oxoisoindolin-5-yl)methyl)carbamate

To a solution of 3-(6-(aminomethyl)-1-oxoisoindolin-2-yl)piperidine-2,6-dione (429 mg, 1.39 mmol, 0.882 eq, concentrated hydrochloric acid) in dichloromethane (24-1) (10.0 mL) were added N,N-diisopropylethylamine (406 mg, 3.14 mmol, 547 uL, 2.00 eq) and 4-(trifluoromethoxy)benzyl carbonochloridate (400 mg, 1.57 mmol, 1.00 eq). The mixture was stirred at 25° C. for 2 h. The mixture was filtered and the filtrate was concentrated under reduced pressure to give a residue. The residue was purified by Prep-HPLC (column: Phenomenex Luna C18 150*25 mm*10 um; mobile phase: [water (formic acid)-acetonitrile]; B %: 30%-60%, 8 min) and lyophilized to afford 4-(trifluoromethoxy)benzyl ((2-(2,6-dioxopiperidin-3-yl)-3-oxoisoindolin-5-yl)methyl)carbamate (9.31 mg, 18.76 umol, 1% yield, 99% purity) as a yellow solid.

1H NMR (400 MHz, DMSO-d6) δ=11.02-10.93 (m, 1H), 7.97 (br t, J=5.7 Hz, 1H), 7.63 (s, 1H), 7.58-7.45 (m, 4H), 7.37 (br d, J=8.1 Hz, 2H), 5.16-5.09 (m, 1H), 5.08 (s, 2H), 4.47-4.40 (m, 1H), 4.32 (br d, J=4.3 Hz, 2H), 4.30-4.26 (m, 1H), 2.96-2.86 (m, 1H), 2.62 (br d, J=1.0 Hz, 1H), 2.43-2.31 (m, 1H), 2.04-1.96 (m, 1H). MS (ESI) m/z 492.0 [M+H]+.

### Example 25. Syntheses of Compound 70

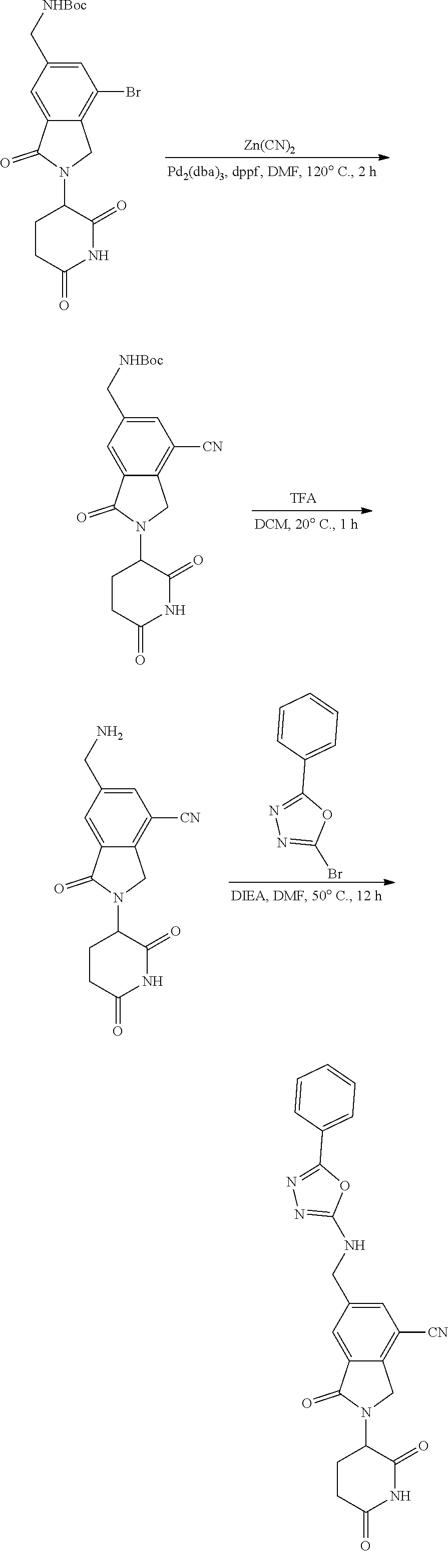 [SMILES: CC(C)(C)OC(=O)NCc1cc(Br)c2c(c1)C(=O)N(C1CCC(=O)NC1=O)C2 ; "Zn(CN)2" ; CC(C)(C)OC(=O)NCc1cc(C#N)c2c(c1)C(=O)N(C1CCC(=O)NC1=O)C2 ; N#Cc1cc(CN)cc2c1CN(C1CCC(=O)NC1=O)C2=O ; Brc1nnc(-c2ccccc2)o1 ; N#Cc1cc(CNc2nnc(-c3ccccc3)o2)cc2c1CN(C1CCC(=O)NC1=O)C2=O]

### Step 1. Procedure for Compound 2-tert-butyl ((7-cyano-2-(2,6-dioxopiperidin-3-yl)-3-oxoisoindolin-5-yl)methyl)carbamate

To a solution of tert-butyl ((7-bromo-2-(2,6-dioxopiperidin-3-yl)-3-oxoisoindolin-5-yl)methyl)carbamate (25-1) (200 mg, 442 umol, 1.00 eq) in N,N-dimethyl formamide (2.00 mL) were added zinc cyanide (199 mg, 1.70 mmol, 108 uL, 3.85 eq), 1,1′bis(diphenylphosphino)ferrocene (49.0 mg, 88.4 umol, 0.200 eq) and tris(dibenzylideneacetone)dipalladium(0) (40.4 mg, 44.2 umol, 0.100 eq). The reaction mixture was stirred at 120° C. for 2 h under nitrogen atmosphere. The reaction mixture was filtered. The filtrate was diluted with water (50 mL) and extracted with ethyl acetate (3×30 mL). The combined organic layers were washed with water (50 mL), dried over anhydrous sodium sulfate, filtered and concentrated under reduced pressure to give a residue. The residue was purified by reversed phase chromatography (C18, 40 g; condition: water/acetonitrile=100:0 to 0:100, 0.1% formic acid) to afford tert-butyl ((7-cyano-2-(2,6-dioxopiperidin-3-yl)-3-oxoisoindolin-5-yl)methyl)carbamate (144 mg, 361 umol, 81% yield) as a white solid.

1H NMR (400 MHz, DMSO-d6) δ=11.03 (br s, 1H), 7.97 (s, 1H), 7.91 (s, 1H), 7.60 (br t, J=6.1 Hz, 1H), 5.14 (dd, J=5.0, 13.3 Hz, 1H), 4.73-4.65 (m, 1H), 4.55-4.47 (m, 1H), 4.27 (br d, J=6.0 Hz, 2H), 2.97-2.84 (m, 1H), 2.60 (td, J=1.8, 15.2 Hz, 1H), 2.47-2.41 (m, 1H), 2.05-1.96 (m, 1H), 1.39 (s, 9H).

### Step 2. Procedure for Compound 3-6-(aminomethyl)-2-(2,6-dioxopiperidin-3-yl)-1-oxoisoindoline-4-carbonitrile

To a solution of tert-butyl ((7-cyano-2-(2,6-dioxopiperidin-3-yl)-3-oxoisoindolin-5-yl)methyl)carbamate (25-2) (144 mg, 361 umol, 1.00 eq) in dichloromethane (5.00 mL) was added trifluoroacetic acid (1.54 g, 13.5 mmol, 1.00 mL, 37.3 eq). The reaction mixture was stirred at 20° C. for 1 h. The reaction mixture was concentrated under reduced pressure to afford 6-(aminomethyl)-2-(2,6-dioxopiperidin-3-yl)-1-oxoisoindoline-4-carbonitrile (107 mg, 358 umol, 99% yield) as a yellow solid. MS (ESI) m/z.299.0 [M+H]+.

### Step 3. Procedure for 2-(2,6-dioxopiperidin-3-yl)-1-oxo-6-(((5-phenyl-1,3,4-oxadiazol-2-yl)amino)methyl)isoindoline-4-carbonitrile

To a solution of 6-(aminomethyl)-2-(2,6-dioxopiperidin-3-yl)-1-oxoisoindoline-4-carbonitrile (25-3) (54.0 mg, 181 umol, 1.00 eq) in N,N-dimethyl formamide (1.00 mL) were added N,N-diisopropylethylamine (46.7 mg, 362 umol, 63.0 uL, 2.00 eq) and 2-bromo-5-phenyl-1,3,4-oxadiazole (40.7 mg, 181. umol, 1.00 eq). The reaction mixture was stirred at 50° C. for 12 h. The residue was dissolved in N,N-dimethyl formamide (0.5 mL) and then filtered. The filtrate was purified by reversed phase chromatography (C18, 40 g; condition: water/acetonitrile=100:0 to 0:100, water) and lyophilized to afford 2-(2,6-dioxopiperidin-3-yl)-1-oxo-6-(((5-phenyl-1,3,4-oxadiazol-2-yl)amino)methyl)isoindoline-4-carbonitrile (14.64 mg, 32.10 umol, 17% yield, 97% purity) as a white solid.

1H NMR (400 MHz, DMSO-d6) δ=11.01 (br s, 1H), 8.51 (t, J=6.1 Hz, 1H), 8.16 (s, 1H), 8.09 (d, J=0.9 Hz, 1H), 7.84-7.79 (m, 2H), 7.55-7.50 (m, 3H), 5.15 (dd, J=5.1, 13.2 Hz, 1H), 4.74-4.66 (m, 1H), 4.62 (d, J=6.1 Hz, 2H), 4.55-4.48 (m, 1H), 2.96-2.85 (m, 1H), 2.60 (td, J=2.0, 15.3 Hz, 1H), 2.48-2.39 (m, 1H), 2.04-1.96 (m, 1H). MS (ESI) m/z.443.1 [M+H]+.

### Example 26. Syntheses of Compound 71

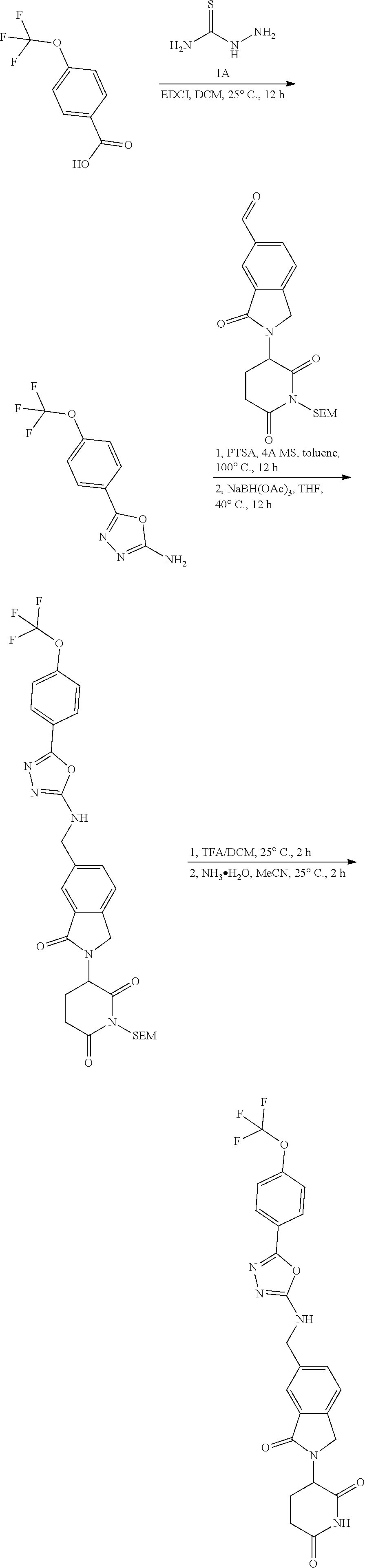 [SMILES: O=C(O)c1ccc(OC(F)(F)F)cc1 ; NNC(N)=S ; Nc1nnc(-c2ccc(OC(F)(F)F)cc2)o1 ; C[Si](C)(C)CCOCN1C(=O)CCC(N2Cc3ccc(C=O)cc3C2=O)C1=O ; C[Si](C)(C)CCOCN1C(=O)CCC(N2Cc3ccc(CNc4nnc(-c5ccc(OC(F)(F)F)cc5)o4)cc3C2=O)C1=O ; O=C1CCC(N2Cc3ccc(CNc4nnc(-c5ccc(OC(F)(F)F)cc5)o4)cc3C2=O)C(=O)N1]

### Step 1. Procedure for preparation of Compound 2-5-(4-(trifluoromethoxy)phenyl)-1,3,4-oxadiazol-2-amine

To a solution of 4-(trifluoromethoxy)benzoic acid (1.30 g, 6.31 mmol, 1.00 eq) in dichloromethane (26-1) (10.0 mL) were added hydrazinecarbothioamide (920 mg, 10.1 mmol, 1.60 eq) and 1-(3-dimethylaminopropyl)-3-ethylcarbodiimide hydrochloride (3.63 g, 18.9 mmol, 3.00 eq). The mixture was stirred at 25° C. for 12 h. The reaction mixture was quenched by addition saturated ammonium chloride (5 mL) at 0° C. and extracted with ethyl acetate (2×10 mL). The combined organic layers were dried over sodium sulfate, filtered and concentrated under reduced pressure to give a residue. The residue was purified by reverse phase chromatography (C18, 40 g; condition: water/acetonitrile=100:0 to 0:100, 0.1% formic acid) and lyophilized to afford 5-(4-(trifluoromethoxy)phenyl)-1,3,4-oxadiazol-2-amine (350 mg, 1.43 mmol, 23% yield) as a gray solid. 1H NMR (400 MHz, DMSO-d6) δ=7.97-7.87 (m, 2H), 7.54 (d, J=8.0 Hz, 2H), 7.34 (s, 2H).

### Step 2. Procedure for Preparation of Compound 3-3-(1-Oxo-6-(((5-(4-(trifluoromethoxy)phenyl)-1,3,4-oxadiazol-2-yl)amino)methyl)isoindolin-2-yl)-1-((2-(trimethylsilyl)ethoxy)methyl)piperidine-2,6-dione

A solution of 2-(2,6-dioxo-1-((2-(trimethylsilyl)ethoxy)methyl)piperidin-3-yl)-3-oxoisoindoline-5-carbaldehyde (26-2) (140 mg, 348 umol, 1.00 eq), 5-(4-(trifluoromethoxy)phenyl)-1,3,4-oxadiazol-2-amine (102 mg, 417 umol, 1.20 eq) in toluene (8.00 mL) were added 4A MS (140 mg) and 4-methylbenzenesulfonic acid (5.99 mg, 34.8 umol, 0.100 eq). The mixture was stirred at 100° C. for 12 h. Then sodium triacetoxy borohydride (369 mg, 1.74 mmol, 5.00 eq) in tetrahydrofuran (2.00 mL) was added into the mixture. The mixture was stirred at 40° C. for 12 h. The reaction mixture was filtered and the filtrate was concentrated under reduced pressure to give a residue. The residue was purified by reverse phase chromatography (C18, 40 g; condition: water/acetonitrile=100:0 to 0:100, 0.1% formic acid) and lyophilized to afford 3-(1-oxo-6-(((5-(4-(trifluoromethoxy)phenyl)-1,3,4-oxadiazol-2-yl)amino)methyl)isoindolin-2-yl)-1-((2-(trimethylsilyl)ethoxy)methyl)piperidine-2,6-dione (60.0 mg, 76.9 umol, 22% yield, 81% purity) as a white solid. MS (ESI) m/z 632.8 [M+H]+

### Step 3. Procedure for preparation of 3-(1-oxo-6-(((5-(4-(trifluoromethoxy)phenyl)-1,3,4-oxadiazol-2-yl)amino)methyl)isoindolin-2-yl)piperidine-2,6-dione

To a solution of 3-(1-oxo-6-(((5-(4-(trifluoromethoxy)phenyl)-1,3,4-oxadiazol-2-yl)amino)methyl) isoindolin-2-yl)-1-((2-(trimethylsilyl)ethoxy)methyl)piperidine-2,6-dione (26-3) (60.0 mg, 95.0 umol, 81% purity, 1.00 eq) in dichloromethane (3.00 mL) was added trifluoroacetic acid (1.00 mL). The mixture was stirred at 25° C. for 2 h. Ammonium hydroxide (5.00 mL) and acetonitrile (5.00 mL) was added into the mixture. The mixture was stirred at 25° C. for 1 h. The reaction mixture was cooled to 25° C. and concentrated under reduced pressure to give a residue. The residue was purified by reverse phase chromatography (C18, 40 g; condition: water/acetonitrile=100:0 to 0:100, 0.1% formic acid) and lyophilized to afford 3-(1-oxo-6-(((5-(4-(trifluoromethoxy)phenyl)-1,3,4-oxadiazol-2-yl)amino)methyl)isoindolin-2-yl)piperidine-2,6-dione (14.80 mg, 29.22 umol, 30% yield, 99% purity) as a white solid.

1H NMR (400 MHz, DMSO-d6) δ=10.98 (br s, 1H), 8.53 (t, J=6.2 Hz, 1H), 7.99-7.88 (m, 2H), 7.76 (s, 1H), 7.69-7.63 (m, 1H), 7.61-7.57 (m, 1H), 7.53 (d, J=8.2 Hz, 2H), 5.11 (dd, J=5.2, 13.3 Hz, 1H), 4.57 (d, J=6.2 Hz, 2H), 4.49-4.40 (m, 1H), 4.35-4.25 (m, 1H), 2.90 (ddd, J=5.2, 13.6, 17.4 Hz, 1H), 2.61 (br d, J=2.4 Hz, 1H), 2.39 (br dd, J=4.8, 13.3 Hz, 1H), 2.03-1.96 (m, 1H). MS (ESI) m/z 502.3 [M+H]+.

### Example 27. Syntheses of Compound 72

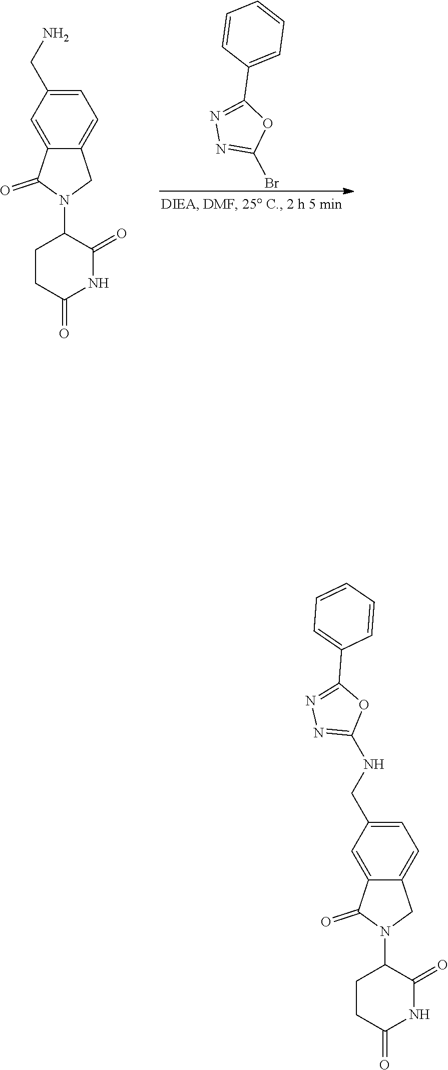 [SMILES: NCc1ccc2c(c1)C(=O)N(C1CCC(=O)NC1=O)C2 ; Brc1nnc(-c2ccccc2)o1 ; O=C1CCC(N2Cc3ccc(CNc4nnc(-c5ccccc5)o4)cc3C2=O)C(=O)N1]

### Step 1. Procedure for preparation of 3-(1-oxo-6-(((5-phenyl-1,3,4-oxadiazol-2-yl)amino)methyl)isoindolin-2-yl)piperidine-2,6-dione

To a solution of 3-(6-(aminomethyl)-1-oxoisoindolin-2-yl)piperidine-2,6-dione (100 mg, 323 umol, 1.00 eq, hydrochloride) in dimethylformamide (2.00 mL) were added N,N-diisopropylethylamine (125 mg, 969 umol, 169 uL, 3.00 eq) and stirred at 25° C. for 5 min. Then was added 2-bromo-5-phenyl-1,3,4-oxadiazole (79.9 mg, 355 umol, 1.10 eq). The mixture was stirred at 25° C. for 2 h. The reaction mixture was filtered and the filtrate was concentrated under reduced pressure to give a residue. The residue was purified by reverse phase chromatography (C18, 40 g; condition: water/acetonitrile=100:0 to 0:100, 0.1% formic acid) and lyophilized to afford 3-(1-oxo-6-(((5-phenyl-1,3,4-oxadiazol-2-yl)amino)methyl)isoindolin-2-yl)piperidine-2,6-dione (13.99 mg, 33.1 umol, 10% yield, 99% purity) as a white solid.

1H NMR (400 MHz, DMSO-d6) δ=10.98 (br s, 1H), 8.46 (t, J=6.4 Hz, 1H), 7.85-7.79 (m, 2H), 7.76 (s, 1H), 7.70-7.63 (m, 1H), 7.62-7.57 (m, 1H), 7.56-7.47 (m, 3H), 5.11 (dd, J=5.0, 13.3 Hz, 1H), 4.56 (d, J=6.4 Hz, 2H), 4.48-4.40 (m, 1H), 4.36-4.27 (m, 1H), 2.90 (ddd, J=5.6, 13.6, 17.4 Hz, 1H), 2.63-2.57 (m, 1H), 2.41-2.33 (m, 1H), 2.03-1.96 (m, 1H). MS (ESI) m/z 418.2 [M+H]+.

### Example 28. Synthesis of Compound 73

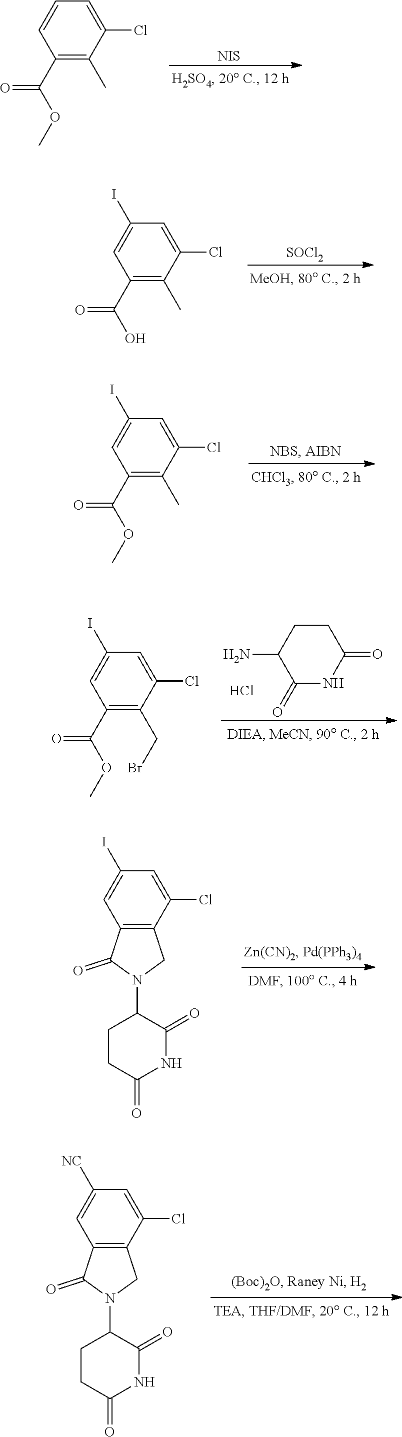

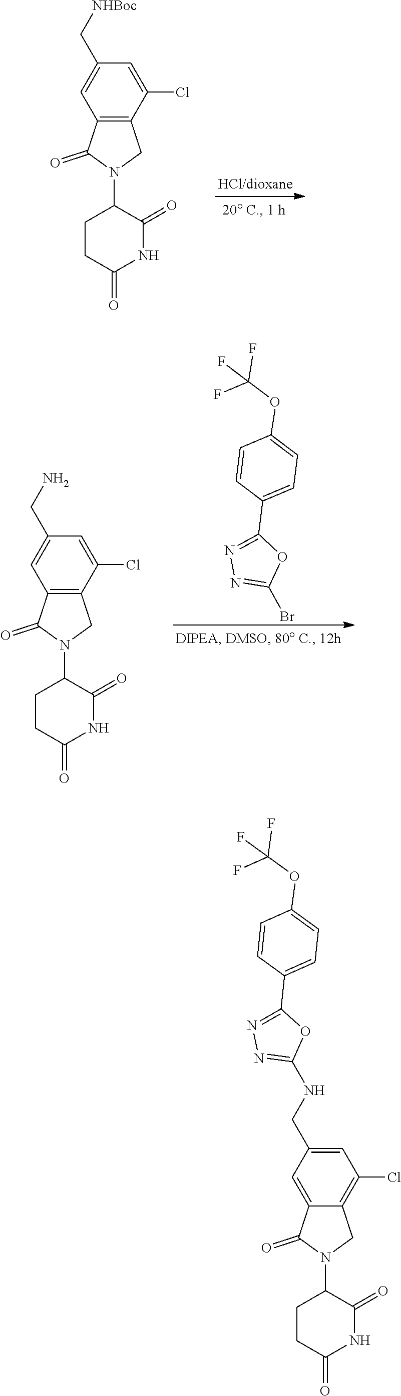 [SMILES: CC(C)(C)OC(=O)NCc1cc(Cl)c2c(c1)C(=O)N(C1CCC(=O)NC1=O)C2 ; NCc1cc(Cl)c2c(c1)C(=O)N(C1CCC(=O)NC1=O)C2 ; FC(F)(F)Oc1ccc(-c2nnc(Br)o2)cc1 ; O=C1CCC(N2Cc3c(Cl)cc(CNc4nnc(-c5ccc(OC(F)(F)F)cc5)o4)cc3C2=O)C(=O)N1]

### Step 1. Procedure for preparation of Compound 2-3-chloro-5-iodo-2-methylbenzoic acid

To a solution of methyl 3-chloro-2-methylbenzoate (4.20 g, 22.7 mmol, 1.00 eq) in sulfuric acid (40.0 mL) was added 1-iodopyrrolidine-2,5-dione (5.12 g, 22.7 mmol, 1.00 eq). The reaction was stirred at 20° C. for 12 h. The reaction was added ice water (200 mL) and extracted with ethyl acetate (3×100 mL) and the organic layers were washed with brine (100 mL), dried over sodium sulfate, filtered and concentrated under reduced pressure to give a residue. The residue was purified by column chromatography (SiO2, petroleum ether/ethyl acetate=10/1 to 5/1) to afford 3-chloro-5-iodo-2-methylbenzoic acid (2.20 g, 7.42 mmol, 33% yield) as a white solid. 1H NMR (400 MHz, CDCl3) δ=8.12 (d, J=1.6 Hz, 1H), 7.89-7.79 (m, 1H), 2.55 (s, 3H).

### Step 2. Procedure for preparation of Compound 3-methyl 3-chloro-5-iodo-2-methylbenzoate

To a solution of 3-chloro-5-iodo-2-methylbenzoic acid (2.20 g, 7.42 mmol, 1.00 eq) in methanol (20.0 mL) was added thionyl chloride (1.77 g, 14.8 mmol, 1.08 mL, 2.00 eq). The reaction was stirred at 80° C. for 2 h. The reaction was diluted with water (100 mL) and extracted with ethyl acetate (3×100 mL). The organic layers were washed with brine (100 mL), dried over sodium sulfate, filtered and concentrated under reduced pressure to give methyl 3-chloro-5-iodo-2-methylbenzoate (2.00 g, 6.44 mmol, 87% yield) as yellow oil. 1H NMR (400 MHz, CDCl3) δ=7.94 (d, J=1.6 Hz, 1H), 7.77 (d, J=1.6 Hz, 1H), 3.83 (s, 3H), 2.47 (s, 3H).

### Step 3. Procedure for preparation of Compound 4-methyl 2-(bromomethyl)-3-chloro-5-iodobenzoate

To a solution of methyl 3-chloro-5-iodo-2-methylbenzoate (2.00 g, 6.44 mmol, 1.00 eq) in trichloromethane (20.0 mL) was added N-Bromosuccinimide (1.26 g, 7.08 mmol, 1.10 eq) and (E)-2,2′-(diazene-1,2-diyl)bis(2-methylpropanenitrile) (1.06 g, 6.44 mmol, 1.00 eq). The reaction was stirred at 80° C. for 2 h. The reaction was diluted with water (100 mL) and extracted with ethyl acetate (3×100 mL). The organic layers were washed with brine (100 mL), dried over sodium sulfate, filtered and concentrated under reduced pressure to give methyl 2-(bromomethyl)-3-chloro-5-iodobenzoate (2.50 g, 6.42 mmol, 99% yield) as yellow oil.

### Step 4. Procedure for Preparation of Compound 5-3-(4-chloro-6-iodo-1-oxoisoindolin-2-yl)piperidine-2,6-dione

To a solution of methyl 2-(bromomethyl)-3-chloro-5-iodobenzoate (2.50 g, 6.42 mmol, 1.00 eq) in acetonitrile (20.0 mL) was added 3-aminopiperidine-2,6-dione hydrochloride (1.27 g, 7.70 mmol, 1.20 eq, hydrochloric acid) and N,N-diisopropylethylamine (2.49 g, 19.2 mmol, 3.35 mL, 3.00 eq). The reaction was stirred at 90° C. for 2 h. The reaction was concentrated under reduced pressure to give a residue. The residue was triturated with ethyl acetate (10.0 mL) and triturated with water (10.0 mL) to afford 3-(4-chloro-6-iodo-1-oxoisoindolin-2-yl)piperidine-2,6-dione (850 mg, 2.10 mmol, 33% yield) as a white solid.

1H NMR (400 MHz, DMSO-d6) δ=11.03 (s, 1H), 8.13 (d, J=1.2 Hz, 1H), 8.03 (d, J=1.2 Hz, 1H), 5.13 (dd, J=5.2, 13.2 Hz, 1H), 4.52-4.39 (m, 1H), 4.34-4.23 (m, 1H), 2.95-2.87 (m, 1H), 2.60 (br d, J=18.0 Hz, 1H), 2.44-2.38 (m, 1H), 2.07-1.99 (m, 1H).

### Step 5. Procedure for Preparation of Compound 6-7-chloro-2-(2,6-dioxopiperidin-3-yl)-3-oxoisoindoline-5-carbonitrile

To a solution of 3-(4-chloro-6-iodo-1-oxoisoindolin-2-yl)piperidine-2,6-dione (800 mg, 1.98 mmol, 1.00 eq) in dimethylformamide (10.0 mL) was added tetrakis[triphenylphosphine]palladium(0) (228 mg, 197 umol, 0.100 eq) and zinc cyanide (163 mg, 1.39 mmol, 88.1 uL, 0.700 eq). The reaction was stirred at 100° C. for 4 h under nitrogen atmosphere. The reaction was added into water (10.0 mL) and filtered to give a filter cake. The filter cake was triturated with ethyl acetate (2.00 mL) for 2 mins to afford 7-chloro-2-(2,6-dioxopiperidin-3-yl)-3-oxoisoindoline-5-carbonitrile (530 mg, 1.75 mmol, 88% yield) as a white solid.

1H NMR (400 MHz, DMSO-d6) δ=11.04 (s, 1H), 8.36 (d, J=1.2 Hz, 1H), 8.25 (s, 1H), 5.17 (dd, J=5.2, 13.2 Hz, 1H), 4.67-4.59 (m, 1H), 4.51-4.41 (m, 1H), 2.95-2.88 (m, 1H), 2.69-2.63 (m, 1H), 2.49-2.42 (m, 1H), 2.06-2.00 (m, 1H).

### Step 6. Procedure for Preparation of Compound 7-tert-butyl ((7-chloro-2-(2,6-dioxopiperidin-3-yl)-3-oxoisoindolin-5-yl)methyl)carbamate

To a solution of 7-chloro-2-(2,6-dioxopiperidin-3-yl)-3-oxoisoindoline-5-carbonitrile (430 mg, 1.42 mmol, 1.00 eq) in tetrahydrofuran (3.00 mL) and dimethylformamide (200 uL) was added triethylamine (1.43 g, 14.1 mmol, 1.97 mL, 10.0 eq), di-tert-butyl dicarbonate (927 mg, 4.25 mmol, 975 uL, 3.00 eq) and raney nickel (40.0 mg). The reaction was stirred at 20° C. for 12 h under hydrogen atmosphere. The reaction was filtered and concentrated under reduced pressure to give a residue. The residue was purified by reverse phase chromatography (C18, 120 g; condition:water/acetonitrile=100:0 to 60:40, 0.1% formic acid) and lyophilized to afford tert-butyl ((7-chloro-2-(2,6-dioxopiperidin-3-yl)-3-oxoisoindolin-5-yl)methyl)carbamate (370 mg, 907 umol, 64% yield) as a white solid.

1H NMR (400 MHz, DMSO-d6) δ=11.01 (br s, 1H), 7.59 (d, J=5.6 Hz, 2H), 7.57-7.47 (m, 1H), 5.13 (dd, J=5.2, 13.2 Hz, 1H), 4.52-4.43 (m, 1H), 4.36-4.28 (m, 1H), 4.24 (br d, J=6.0 Hz, 2H), 2.97-2.85 (m, 1H), 2.62 (br s, 1H), 2.48-2.41 (m, 1H), 2.06-1.98 (m, 1H), 1.40 (s, 9H).

### Step 7 Procedure for preparation of Compound 8-3-(6-(aminomethyl)-4-chloro-1-oxoisoindolin-2-yl)piperidine-2,6-dione

To a solution of tert-butyl ((7-chloro-2-(2,6-dioxopiperidin-3-yl)-3-oxoisoindolin-5-yl)methyl)carbamate (150 mg, 367 umol, 1.00 eq) in dichloromethane (2.00 mL) was added trifluoroacetic acid (200 uL). The mixture was stirred at 20° C. for 1 h. The reaction was concentrated under reduced pressure to give 3-(6-(aminomethyl)-4-chloro-1-oxoisoindolin-2-yl)piperidine-2,6-dione (110 mg, 357.45 umol, 97.19% yield) as a white solid. MS (ESI) m/z 308.0 [M+H]+.

### Step 8. Procedure for Preparation of 3-(4-chloro-1-oxo-6-(((5-(4-(trifluoromethoxy)phenyl)-1,3,4-oxadiazol-2-yl)amino)methyl)isoindolin-2-yl)piperidine-2,6-dione

To a solution of 3-(6-(aminomethyl)-4-chloro-1-oxoisoindolin-2-yl)piperidine-2,6-dione (110 mg, 357 umol, 1.00 eq) and 2-bromo-5-(4-(trifluoromethoxy)phenyl)-1,3,4-oxadiazole (132 mg, 428 umol, 1.20 eq) in dimethylsulfoxide (2.00 mL) was added N,N-diisopropylethylamine (138 mg, 1.07 mmol, 186 uL, 3.00 eq). The reaction was stirred at 80° C. for 12 h. The reaction was filtered to give a residue. The residue was purified by Prep-HPLC (column: Phenomenex luna C18 150*25 mm*10 um; mobile phase: [water(FA)-ACN]; B %: 35%-65%, 10 min) to afford 3-(4-chloro-1-oxo-6-(((5-(4-(trifluoromethoxy)phenyl)-1,3,4-oxadiazol-2-yl)amino)methyl)isoindolin-2-yl)piperidine-2,6-dione (59.48 mg, 99.90 umol, 27.95% yield, 90% purity) as a white solid.

1H NMR (400 MHz, DMSO-d6) δ=11.00 (s, 1H), 8.56 (t, J=6.0 Hz, 1H), 7.94 (d, J=8.8 Hz, 2H), 7.77 (s, 2H), 7.54 (br d, J=8.4 Hz, 2H), 5.13 (dd, J=5.2, 13.2 Hz, 1H), 4.59 (br d, J=6.0 Hz, 2H), 4.53-4.43 (m, 1H), 4.37-4.27 (m, 1H), 2.91 (br s, 1H), 2.62 (br s, 1H), 2.47 (br s, 1H), 2.07-1.98 (m, 1H). 19F NMR (376 MHz, DMSO-d6) δ=−56.71 (s, 1F). MS (ESI) m/z 536.0 [M+H]+.

### Example 29. Synthesis of Compound 74

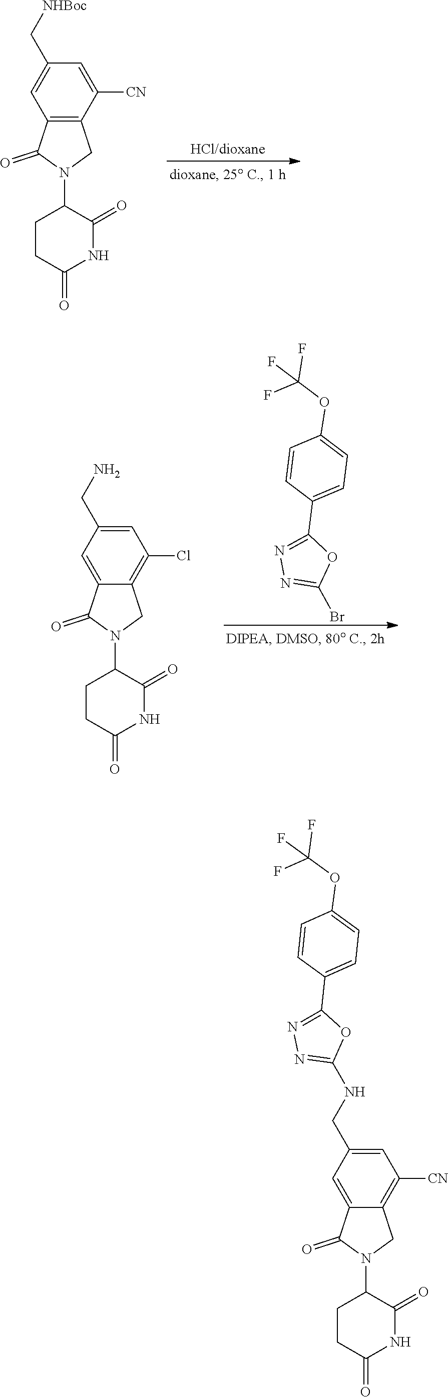 [SMILES: CC(C)(C)OC(=O)NCc1cc(C#N)c2c(c1)C(=O)N(C1CCC(=O)NC1=O)C2 ; NCc1cc(Cl)c2c(c1)C(=O)N(C1CCC(=O)NC1=O)C2 ; FC(F)(F)Oc1ccc(-c2nnc(Br)o2)cc1 ; N#Cc1cc(CNc2nnc(-c3ccc(OC(F)(F)F)cc3)o2)cc2c1CN(C1CCC(=O)NC1=O)C2=O]

### Step 1. Procedure for Compound 2-6-(aminomethyl)-2-(2,6-dioxopiperidin-3-yl)-1-oxoisoindoline-4-carbonitrile

To a solution of tert-butyl ((7-cyano-2-(2,6-dioxopiperidin-3-yl)-3-oxoisoindolin-5-yl)methyl)carbamate (100 mg, 251 umol, 1.00 eq) in dioxane (1.00 mL) was added hydrochloric acid/dioxane (4.00 M, 1.00 mL). The reaction mixture was stirred at 25° C. for 1 h. The reaction mixture was concentrated under reduced pressure to afford 6-(aminomethyl)-2-(2,6-dioxopiperidin-3-yl)-1-oxoisoindoline-4-carbonitrile (105 mg, crude) as a white solid.

1H NMR (400 MHz, DMSO-d6) δ=11.03 (s, 1H), 8.52 (br s, 2H), 8.26 (d, J=1.2 Hz, 1H), 8.24 (s, 1H), 5.15 (dd, J=5.2, 13.2 Hz, 1H), 4.76 (d, J=18.2 Hz, 1H), 4.60-4.52 (m, 1H), 4.22 (br d, J=5.2 Hz, 2H), 2.98-2.85 (m, 1H), 2.61 (br d, J=17.8 Hz, 1H), 2.49-2.40 (m, 1H), 2.06-1.98 (m, 1H).

### Step 2. Procedure for 2-(2,6-dioxopiperidin-3-yl)-1-oxo-6-(((5-(4-(trifluoromethoxy)phenyl)-1,3,4-oxadiazol-2-yl)amino)methyl)isoindoline-4-carbonitrile

To a solution of 6-(aminomethyl)-2-(2,6-dioxopiperidin-3-yl)-1-oxoisoindoline-4-carbonitrile (48.2 mg, 161 umol, 1.00 eq) in dimethylsulfoxide (1.00 mL) was added diisopropylethylamine (41.8 mg, 323 umol, 56.3 uL, 2.00 eq) and 2-bromo-5-(4-(trifluoromethoxy)phenyl)-1,3,4-oxadiazole (50.0 mg, 161 umol, 1.00 eq). The reaction mixture was stirred at 80° C. for 2 h. The reaction mixture was dissolved in acetonitrile (0.500 mL) and then filtered. The filtrate was purified by Prep-HPLC(column: Phenomenex C18 75*30 mm*3 um; mobile phase: [water(formic acid)-acetonitrile]; B %: 30%-60%, 7 min) and lyophilized to afford 2-(2,6-dioxopiperidin-3-yl)-1-oxo-6-(((5-(4-(trifluoromethoxy)phenyl)-1,3,4-oxadiazol-2-yl)amino)methyl)isoindoline-4-carbonitrile (22.61 mg, 41.23 umol, 25% yield, 96% purity) as an off-white solid.

1H NMR (400 MHz, DMSO-d6) δ=11.00 (br d, J=6.2 Hz, 1H), 8.58 (t, J=6.2 Hz, 1H), 8.16 (s, 1H), 8.08 (s, 1H), 7.93 (d, J=8.8 Hz, 2H), 7.53 (d, J=8.6 Hz, 2H), 5.14 (dd, J=5.2, 13.2 Hz, 1H), 4.75-4.65 (m, 1H), 4.63 (br d, J=6.0 Hz, 2H), 4.56-4.48 (m, 1H), 2.90 (ddd, J=5.2, 13.6, 17.8 Hz, 1H), 2.64-2.56 (m, 1H), 2.47-2.40 (m, 1H), 2.04-1.95 (m, 1H). MS (ESI) m/z 527.1 [M+H]+.

### Example 30. Synthesis of Compound 75

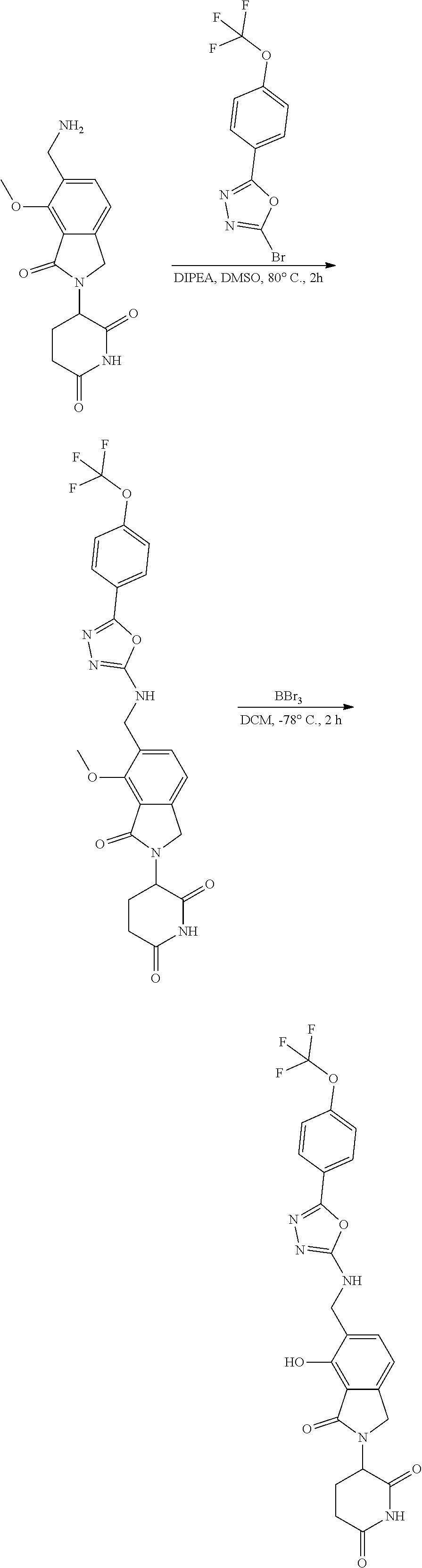 [SMILES: COc1c(CN)ccc2c1C(=O)N(C1CCC(=O)NC1=O)C2 ; FC(F)(F)Oc1ccc(-c2nnc(Br)o2)cc1 ; COc1c(CNc2nnc(-c3ccc(OC(F)(F)F)cc3)o2)ccc2c1C(=O)N(C1CCC(=O)NC1=O)C2 ; "BBr3" ; O=C1CCC(N2Cc3ccc(CNc4nnc(-c5ccc(OC(F)(F)F)cc5)o4)c(O)c3C2=O)C(=O)N1]

### Step 1. Procedure for preparation of Compound 2-3-(7-methoxy-1-oxo-6-(((5-(4-(trifluoromethoxy)phenyl)-1,3,4-oxadiazol-2-yl)amino)methyl)isoindolin-2-yl)piperidine-2,6-dione

To a solution of 3-(6-(aminomethyl)-7-methoxy-1-oxoisoindolin-2-yl)piperidine-2,6-dione (80.0 mg, 264 umol, 1 eq) in dimethylsulfoxide (1.00 mL) was added N,N-diisopropylethylamine (102 mg, 791 umol, 138 uL, 3.00 eq) and 2-bromo-5-(4-(trifluoromethoxy)phenyl)-1,3,4-oxadiazole (65.2 mg, 211 umol, 0.800 eq). The reaction mixture was stirred at 80° C. for 2 h. The reaction mixture was diluted with water (20.0 mL), extracted with ethyl acetate (2×30.0 mL). The combined organic layers was washed with brine (2×20.0 mL), dried over sodium sulfate, filtered and concentrated in under reduced pressure to give a residue. The residue was purified by column chromatography (SiO2, dichloromethane: ethyl acetate=1/0 to 0/1) and concentrated under reduced pressure to afford 3-(7-methoxy-1-oxo-6-(((5-(4-(trifluoromethoxy)phenyl)-1,3,4-oxadiazol-2-yl)amino)methyl) isoindolin-2-yl)piperidine-2,6-dione (90.0 mg, 169 umol, 64% yield) as a white solid.

1H NMR (400 MHz, DMSO-d6) δ=10.97 (s, 1H), 8.37 (t, J=5.9 Hz, 1H), 7.92 (d, J=8.8 Hz, 2H), 7.64 (d, J=7.8 Hz, 1H), 7.53 (d, J=8.5 Hz, 2H), 7.29 (d, J=7.8 Hz, 1H), 5.07 (dd, J=5.1, 13.2 Hz, 1H), 4.52 (d, J=5.8 Hz, 2H), 4.43-4.37 (m, 1H), 4.32-4.23 (m, 1H), 4.04 (s, 3H), 2.94-2.87 (m, 1H), 2.60 (br d, J=13.6 Hz, 1H), 2.38 (br dd, J=4.6, 13.3 Hz, 1H), 2.01 (br d, J=5.3 Hz, 1H).

### Step 2 Procedure for preparation of 3-(7-hydroxy-1-oxo-6-(((5-(4-(trifluoromethoxy)phenyl)-1,3,4-oxadiazol-2-yl)amino)methyl)isoindolin-2-yl)piperidine-2,6-dione

To a solution of 3-[7-methoxy-1-oxo-6-[[[5-[4-(trifluoromethoxy)phenyl]-1,3,4-oxadiazol-2-yl]amino]methyl] isoindolin-2-yl]piperidine-2,6-dione (70.0 mg, 132 umol, 1.00 eq) in dichloromethane (3.00 mL) was added tribromoborane (99.0 mg, 395 umol, 38.1 uL, 3.00 eq) at −78° C. under nitrogen atmosphere. The reaction mixture was stirred at −78° C. for 2 h. The reaction mixture was quenched with water (5.00 mL) at −78° C. and the mixture was concentrated under reduced pressure at 20° C. to give a residue. The residue was purified by reverse phase chromatography (C18, 40 g; condition: water/acetonitrile=100:0 to 0:100, 0.1% formic) and lyophilized to afford 3-(7-hydroxy-1-oxo-6-(((5-(4-(trifluoromethoxy)phenyl)-1,3,4-oxadiazol-2-yl) amino)methyl)isoindolin-2-yl)piperidine-2,6-dione (28.29 mg, 53.04 umol, 40.26% yield, 97% purity) as an off-white solid.

1H NMR (400 MHz, DMSO-d6) δ=11.01 (s, 1H), 9.28 (s, 1H), 8.36 (br t, J=5.7 Hz, 1H), 7.93 (d, J=8.6 Hz, 2H), 7.60-7.48 (m, 3H), 7.04 (d, J=7.5 Hz, 1H), 5.05 (dd, J=5.1, 13.1 Hz, 1H), 4.54-4.46 (m, 2H), 4.46-4.38 (m, 1H), 4.33-4.25 (m, 1H), 2.97-2.85 (m, 1H), 2.61 (br d, J=17.4 Hz, 1H), 2.42 (br dd, J=4.8, 8.4 Hz, 1H), 2.06-1.97 (m, 1H). MS (ESI) m/z 518.1 [M+H]+.

### Example 31. Compound Binding to CRBN by HTRF Assay

Compound activity was monitored in a Homogenous Time-Resolved Fluorescence (HTRF) assay using 1-[5-({2-[2-(2-{[2-(2,6-dioxopiperidin-3-yl)-1,3-dioxo-2,3-dihydro-1H-isoindol-4-yl]oxy}acetamido)ethoxy]ethyl}carbamoyl)pentyl]-3,3-dimethyl-2-[(1E,3E)-5-[(2E)-1,3,3-trimethyl-5-sulfo-2,3-dihydro-1H-indol-2-ylidene]penta-1,3-dien-1-yl]-3H-indol-1-ium-5-sulfonate as a fluorescent probe. Biochemical assays were conducted in Greiner white 384 well HiBase plates (Cat. No 784075-25) in 10 μL total volume. A one pot detection solution of CRBN-DDB1 (2.5 nM), Anti-His Terbium Cryptate Gold (1×, PerkinElmer Cat. #: 61HI2TLB), and Cy5-Thalidomide (100 nM, Tenova Cat.: T52461) was prepared in 20 mM HEPES, 20 mM NaCl, 0.2 mM TCEP, 0.2 mM EDTA, and 0.005% Tween20 was dispensed to each assay plate. Compounds were stored in dry, ambient temperatures at 10 mM. A 10-point, 1:3 dilution series was prepared from 10 mM stock concentrations in Echo-compatible LDV plates. 10 nL of each compound dilution series was dispensed into assays wells using an Echo 650 (Labcyte inc. USA). 10 nL of 10 mM Lenalidomide was transferred into the active-control wells for the assay and 10 nL of DMSO was transferred into the neutral-control wells. The assay was then allowed to incubate for 30 min at ambient temperature after transferring compound. Plate measurements were taken on a Pherastar FSX (BMG Labtech, Germany) using the HTRF Red filter (Ex. 337 nm, em1:620 nm, em2:665 nm) (Flashes: 50, Integration time: 60-400 us, Z-height: 10 mm, Ratio-multipler: 10,000). The HTRF signal was then subsequently normalized to the neutral and active controls. Analysis and IC50 values were derived using KNIME analytics (KNIME Zurich) transformation and fitting within Collaborative Drug Discovery (Collaborative Drug Discovery USA). Ki was derived from the geometric mean of the IC50 values using the Cheng-Prustoff transformation.

### Example 32. HiBiT Assay

HEK293 clonal lines with CRISPR KI HiBiT tag on CDK2 proteins and stably expressing LgBiT protein were obtained from Promega (Madison, WI). Cells were plated at 5000 cells per well using Multiflo (BioTek) in 384-well white solid bottom plates (Corning, 3570BC) in 25 ul volume in DMEM media (DMEM, high glucose, HEPES, no phenol red (ThermoFisher Scientific, 21063029)) containing 10% FBS (Corning, 35-075-CV), 1% Penicillin/Streptomycin (ThermoFisher Scientific, 15140-122). Cells were incubated for 16 hours at 37° C., 5% CO2. 75 nL of a compound at 30 μM were dosed into the plate using an Echo® 650 liquid handler (Labcyte). Cells were incubated at 37° C., 5% CO2 for 24 hours. 5 ul of HiBiT Lysis Reagent (Promega, N3030) is added using Combi Multidrop (ThermoFisher Scientific), plates are incubated for 15′ at 37° C., 5% CO2 and then signal was read on a Pherastar FSX using “LUM plus” optic module.

Analysis was performed in Scinamic (Scinamic, Cambridge, MA). Luminescence response (R) was calculated by the formula: response=100*(S−N)/(P−N) where S is the signal of the well, N and P the mean negative and positive control values respectively of the same plate. The luminescence response was then fitted in Scinamic using a 3-parameter agonist logistic fit (hillslope=1, EC50>0, top/bottom unconstrained).

TABLE 2
HTRF and HiBiT Data

| | CDK2 HiBiT | CRBN HTRF |
|---|---|---|
| Cmpd No. | (Dmax (%)) | (Ki) |
| 1 | B | F |
| 2 | B | F |
| 3 | B | F |
| 4 | B | F |
| 5 | B | F |
| 6 | B | E |
| 7 | B | F |
| 8 | B | F |
| 9 | C | F |
| 10 | C | F |
| 11 | C | F |
| 12 | C | E |
| 13 | B | E |
| 14 | B | F |
| 15 | B | E |
| 16 | B | F |
| 17 | B | F |
| 18 | B | F |
| 19 | B | F |
| 20 | B | F |
| 21 | A | E |
| 22 | B | E |
| 23 | B | F |
| 24 | A | E |
| 25 | B | E |
| 26 | B | F |
| 28 | A | F |
| 29 | A | E |
| 30 | B | F |
| 31 | B | E |
| 32 | A | F |
| 33 | B | E |
| 34 | B | E |
| 35 | B | E |
| 36 | B | F |
| 37 | B | E |
| 38 | B | F |
| 39 | B | E |
| 40 | B | E |
| 41 | B | F |
| 42 | B | E |
| 43 | B | E |
| 44 | B | E |
| 45 | B | E |
| 46 | B | F |
| 47 | B | E |
| 48 | B | E |
| 49 | B | E |
| 50 | B | F |
| 51 | B | F |
| 52 | B | E |
| 53 | A | F |
| 54 | B | F |
| 55 | A | E |
| 56 | B | E |
| 57 | B | E |
| 58 | B | E |
| 59 | B | F |
| 60 | B | E |
| 61 | B | E |
| 62 | B | E |
| 63 | B | E |
| 64 | B | F |
| 65 | B | E |
| 66 | B | E |
| 67 | B | F |
| 68 | A | F |
| 69 | B | F |
| 70 | B | F |
| 71 | C | F |
| 72 | B | E |
| 73 | C | E |
| 74 | B | F |
| 75 | B | F |

For CDK2 HiBiT: According to the code, A represents a Dmax value of ≤10%, B represents a Dmax value of >10% and ≤50%, C represents a Dmax value of >50% and ≤80%, D represents Dmax value of >80%. For HTRF: According to the code, E represents a Ki value of ≤0.1 μM, F represents a Ki value >0.1 μM and ≤1 μM, G represents an Ki value >1 μM.

### EQUIVALENTS

While specific embodiments have been discussed, the above specification is illustrative and not restrictive. Many variations of the embodiments will become apparent to those skilled in the art upon review of this specification. The full scope of what is disclosed should be determined by reference to the claims, along with their full scope of equivalents, and the specification, along with such variations.

What is claimed is:

1. A compound of Formula (I): 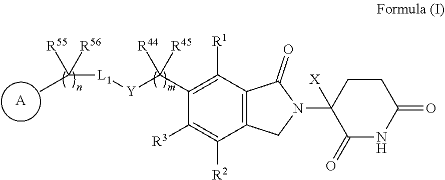
or a pharmaceutically acceptable salt thereof,
wherein:
X is H or deuterium;
Y is NH or O;
L¹ is: 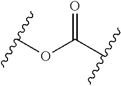 [SMILES: *OC(*)=O]
or a 5-6 membered heteroaryl;
each of R¹, R², R³ is independently H, halogen, cyano, C₁₋₆ alkyl, or hydroxy;
each of R⁴⁴ and R⁴⁵ is independently H or C₁₋₆ alkyl;
each of R⁵⁵ and R⁵⁶ is independently H or C₁₋₆ alkyl;
ring A is aryl or heteroaryl, wherein each of aryl and heteroaryl is optionally substituted with one or more occurrences of R⁴;
each occurrence of R⁴ is independently halogen, cyano, —NO₂, hydroxyl, C₁₋₆ alkyl, C₂₋₆ alkynyl, C₁₋₄ alkoxy, C₁₋₆ thioalkoxy, C₃₋₁₀ cycloalkyl, 3 to 10 membered heterocyclyl, aryl, heteroaryl, or —O-heterocyclyl, wherein each of C₁₋₄ alkyl, C₂₋₆ alkynyl, C₁₋₆ alkoxy, C₁₋₆ thioalkoxy, C₃₋₁₀ cycloalkyl, 3 to 10 membered heterocyclyl, aryl, and heteroaryl is optionally substituted with one or more occurrences of R⁵;
each occurrence of R⁵ is independently halogen, oxo, hydroxy, C₂₋₆ alkynyl, C₁₋₆ alkyl, C₁₋₆ alkoxy, C₁₋₆ thioalkoxy, C₃₋₁₀ cycloalkyl, 3 to 10 membered heterocyclyl, aryl, or heteroaryl, wherein C₁₋₆ alkyl is optionally substituted by one or more occurrences of halogen;
n is 0, 1, 2, or 3; and
m is 1 or 2;
provided that the compound is not:

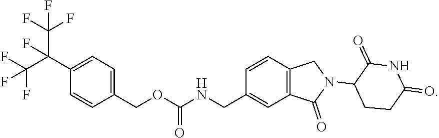 [SMILES: O=C1CCC(N2Cc3ccc(CNC(=O)OCc4ccc(C(F)(C(F)(F)F)C(F)(F)F)cc4)cc3C2=O)C(=O)N1]

2. The compound of claim 1, wherein m is 1 or 2.

3. The compound of claim 1, wherein L¹ is

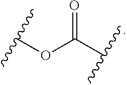 [SMILES: *OC(*)=O]

4. The compound of claim 1, where L¹ is

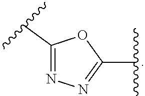 [SMILES: *c1nnc(*)o1]

5. The compound of claim 1, wherein X is H.

6. The compound of claim 1, wherein R¹, R², and R³ are H.

7. The compound of claim 1, wherein R⁵⁵ and R⁵⁶ are H.

8. The compound of claim 1, wherein n is 2, 1, or 0.

9. The compound of claim 1, wherein ring A is selected from the group consisting of:

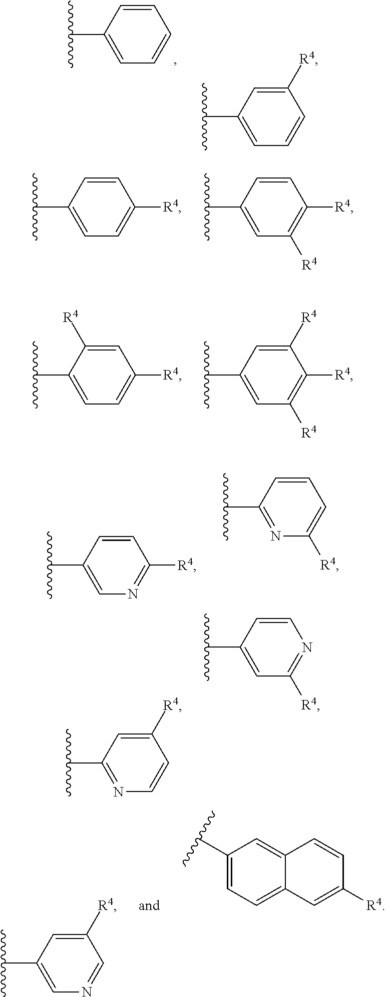

10. The compound of claim 1, wherein ring A is C₆₋₁₀ aryl optionally substituted with one or more occurrences of R⁴.

11. The compound of claim 10, wherein ring A is selected from the group consisting of:

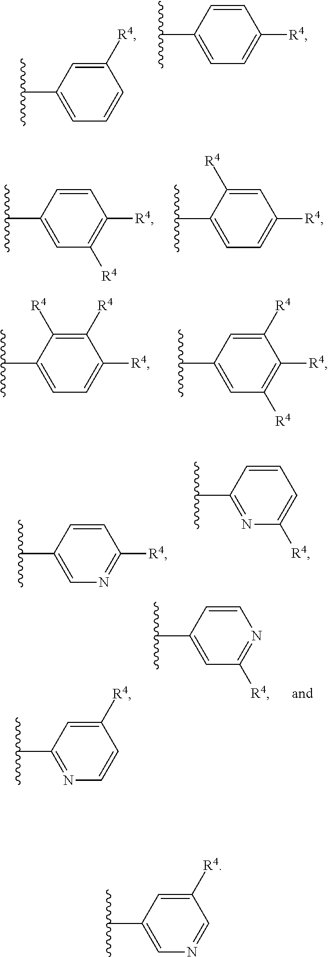

12. The compound of claim 1, wherein ring A is 5-membered heteroaryl or 6-membered heteroaryl, wherein each of 5-membered heteroaryl and 6-membered heteroaryl optionally substituted with one or more occurrences of R⁴.

13. The compound of claim 12, where ring A is selected from the group consisting of:

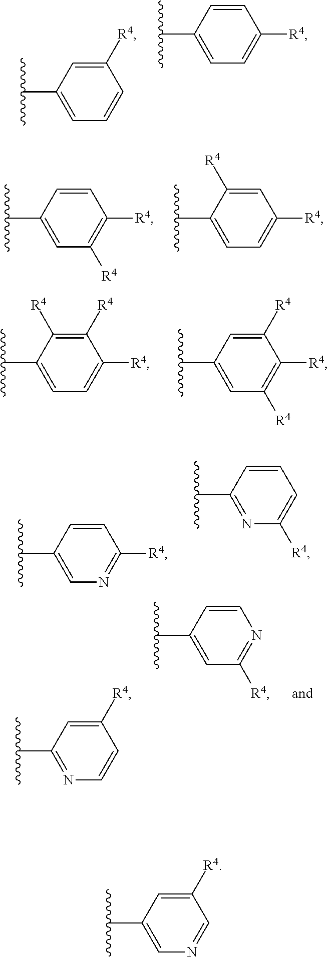

14. The compound of claim 1, wherein R⁴ is selected from the group consisting of: flourine, chlorine, hydroxy, —CH₃, —CF₃, —OCHF₂, —OCH₃, —OCH₂CH₃, —OCF₃, —OCHF₂, —OCH₂CH₃, —OCH₂CF₃, —SCH₃, —SCF₃, —CH2CF₃, —CH2CH2CH₃, —C(CH₃)(CH₃)(CH₃), —CH(CH₃)(CH₃), HC≡C(CF₃)

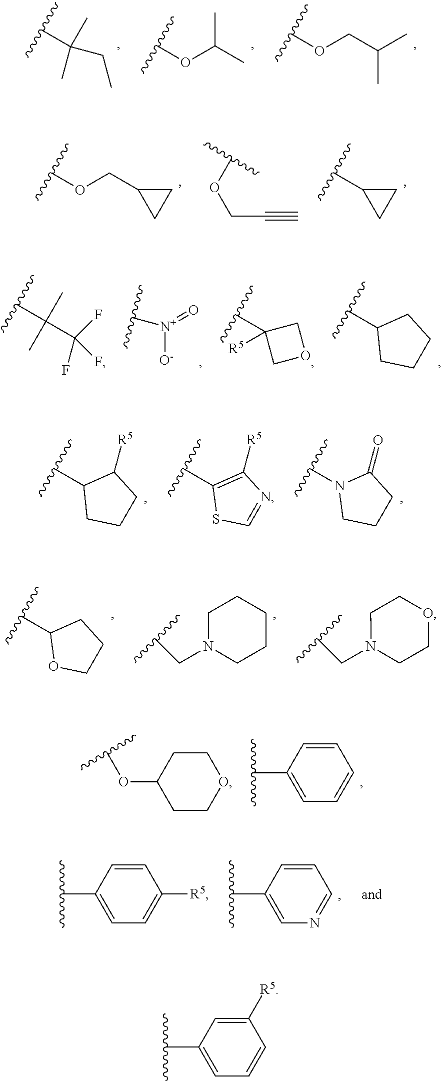

15. The compound of claim 1, wherein R⁵ is selected from the group consisting of: flourine, chlorine, hydroxy, —CH₃, —CF₃, and —OCH₃.

16. A compound selected from the group consisting of: 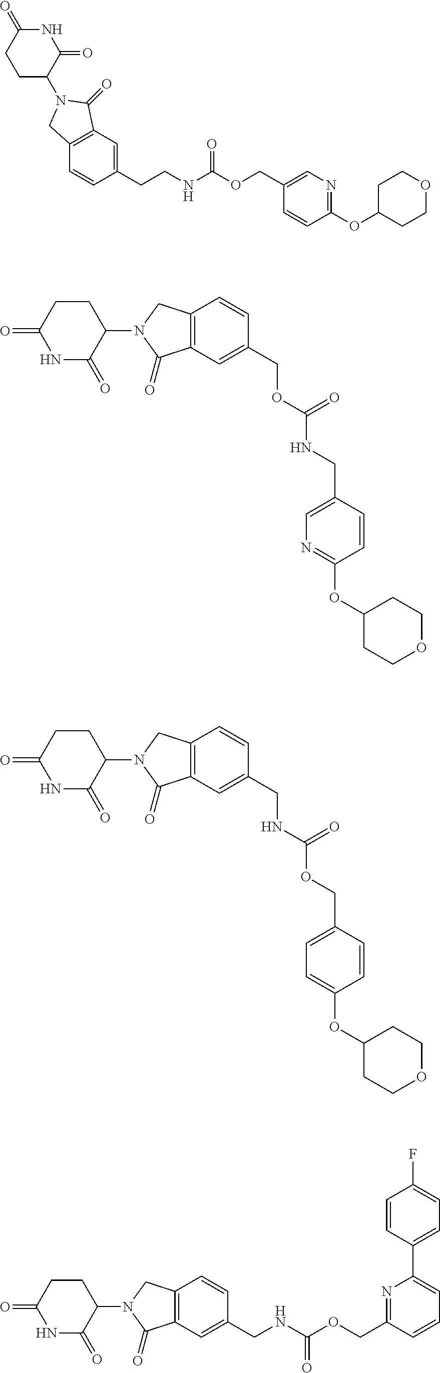 [SMILES: O=C1CCC(N2Cc3ccc(CCNC(=O)OCc4ccc(OC5CCOCC5)nc4)cc3C2=O)C(=O)N1 ; O=C1CCC(N2Cc3ccc(COC(=O)NCc4ccc(OC5CCOCC5)nc4)cc3C2=O)C(=O)N1 ; O=C1CCC(N2Cc3ccc(CNC(=O)OCc4ccc(OC5CCOCC5)cc4)cc3C2=O)C(=O)N1 ; O=C1CCC(N2Cc3ccc(CNC(=O)OCc4cccc(-c5ccc(F)cc5)n4)cc3C2=O)C(=O)N1] 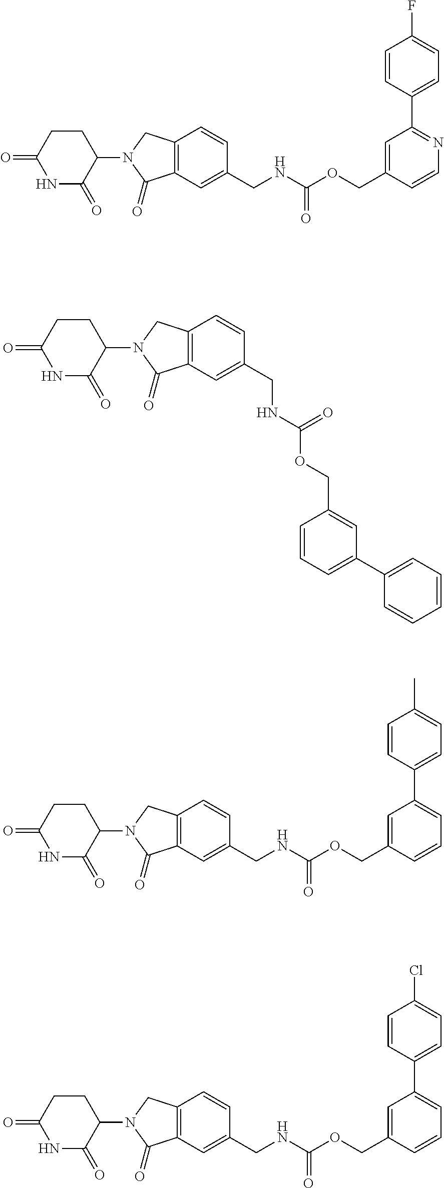 [SMILES: O=C1CCC(N2Cc3ccc(CNC(=O)OCc4ccnc(-c5ccc(F)cc5)c4)cc3C2=O)C(=O)N1 ; O=C1CCC(N2Cc3ccc(CNC(=O)OCc4cccc(-c5ccccc5)c4)cc3C2=O)C(=O)N1 ; Cc1ccc(-c2cccc(COC(=O)NCc3ccc4c(c3)C(=O)N(C3CCC(=O)NC3=O)C4)c2)cc1 ; O=C1CCC(N2Cc3ccc(CNC(=O)OCc4cccc(-c5ccc(Cl)cc5)c4)cc3C2=O)C(=O)N1] 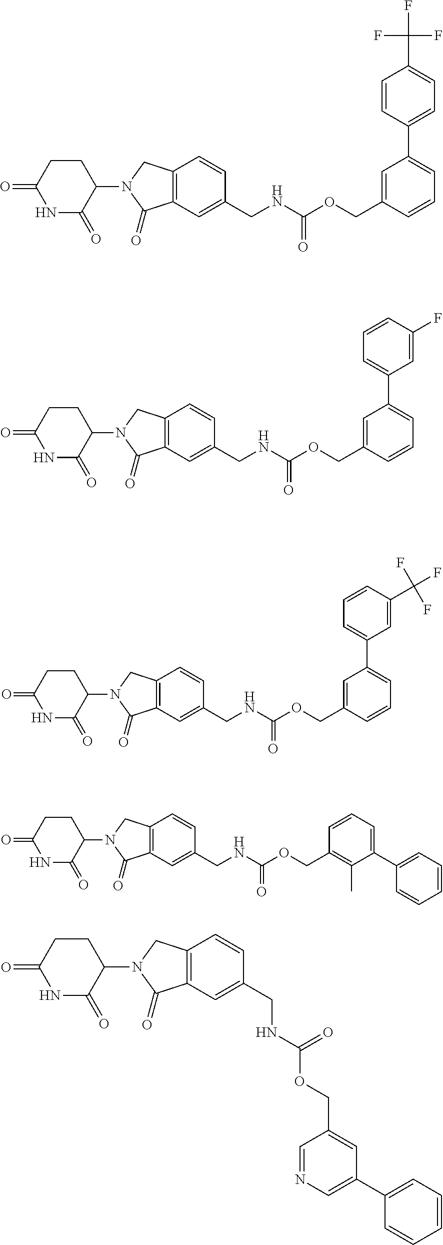 [SMILES: O=C1CCC(N2Cc3ccc(CNC(=O)OCc4cccc(-c5ccc(C(F)(F)F)cc5)c4)cc3C2=O)C(=O)N1 ; O=C1CCC(N2Cc3ccc(CNC(=O)OCc4cccc(-c5cccc(F)c5)c4)cc3C2=O)C(=O)N1 ; O=C1CCC(N2Cc3ccc(CNC(=O)OCc4cccc(-c5cccc(C(F)(F)F)c5)c4)cc3C2=O)C(=O)N1 ; Cc1c(COC(=O)NCc2ccc3c(c2)C(=O)N(C2CCC(=O)NC2=O)C3)cccc1-c1ccccc1 ; O=C1CCC(N2Cc3ccc(CNC(=O)OCc4cncc(-c5ccccc5)c4)cc3C2=O)C(=O)N1] 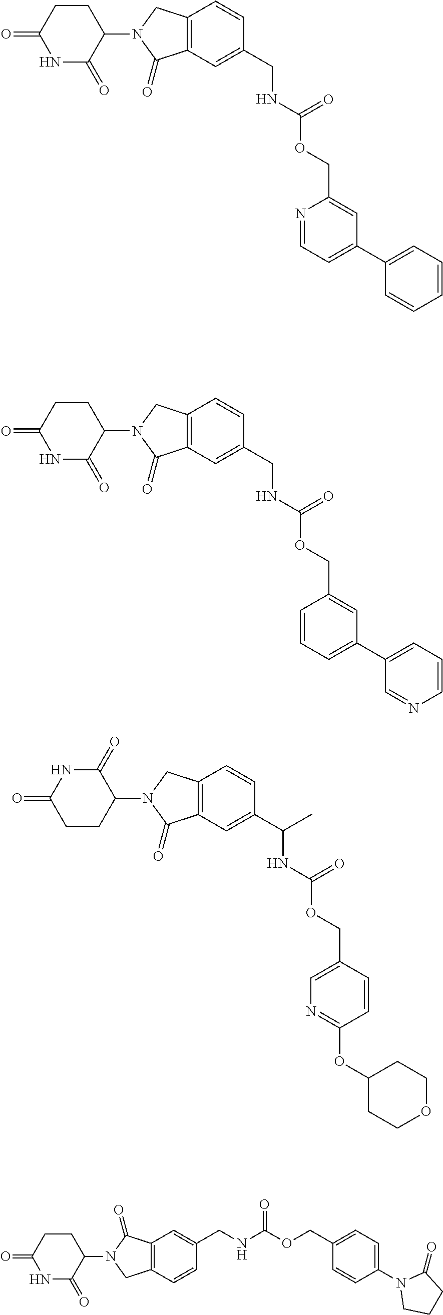 [SMILES: O=C1CCC(N2Cc3ccc(CNC(=O)OCc4cc(-c5ccccc5)ccn4)cc3C2=O)C(=O)N1 ; O=C1CCC(N2Cc3ccc(CNC(=O)OCc4cccc(-c5cccnc5)c4)cc3C2=O)C(=O)N1 ; CC(NC(=O)OCc1ccc(OC2CCOCC2)nc1)c1ccc2c(c1)C(=O)N(C1CCC(=O)NC1=O)C2 ; O=C1CCC(N2Cc3ccc(CNC(=O)OCc4ccc(N5CCCC5=O)cc4)cc3C2=O)C(=O)N1] 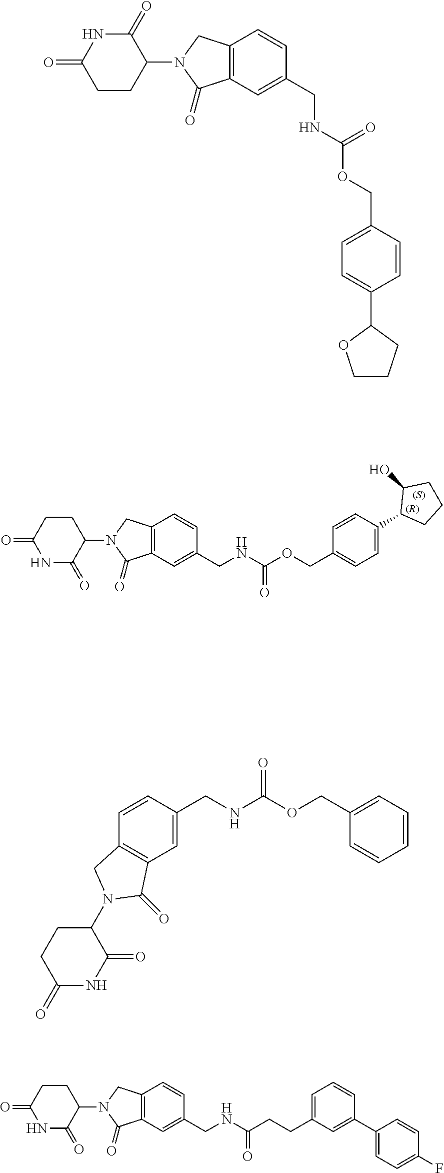 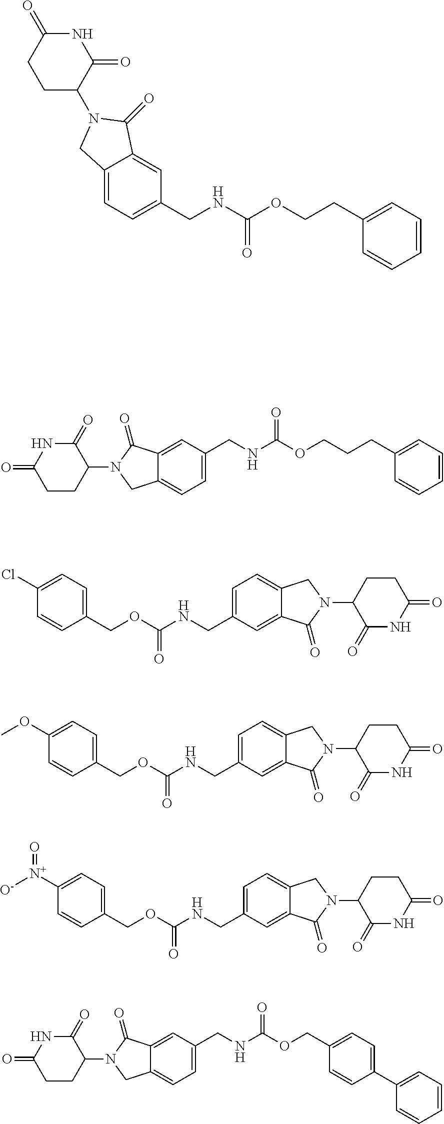 [SMILES: O=C1CCC(N2Cc3ccc(CNC(=O)OCCc4ccccc4)cc3C2=O)C(=O)N1 ; O=C1CCC(N2Cc3ccc(CNC(=O)OCCCc4ccccc4)cc3C2=O)C(=O)N1 ; O=C1CCC(N2Cc3ccc(CNC(=O)OCc4ccc(Cl)cc4)cc3C2=O)C(=O)N1 ; COc1ccc(COC(=O)NCc2ccc3c(c2)C(=O)N(C2CCC(=O)NC2=O)C3)cc1 ; O=C1CCC(N2Cc3ccc(CNC(=O)OCc4ccc([N+](=O)[O-])cc4)cc3C2=O)C(=O)N1 ; O=C1CCC(N2Cc3ccc(CNC(=O)OCc4ccc(-c5ccccc5)cc4)cc3C2=O)C(=O)N1] 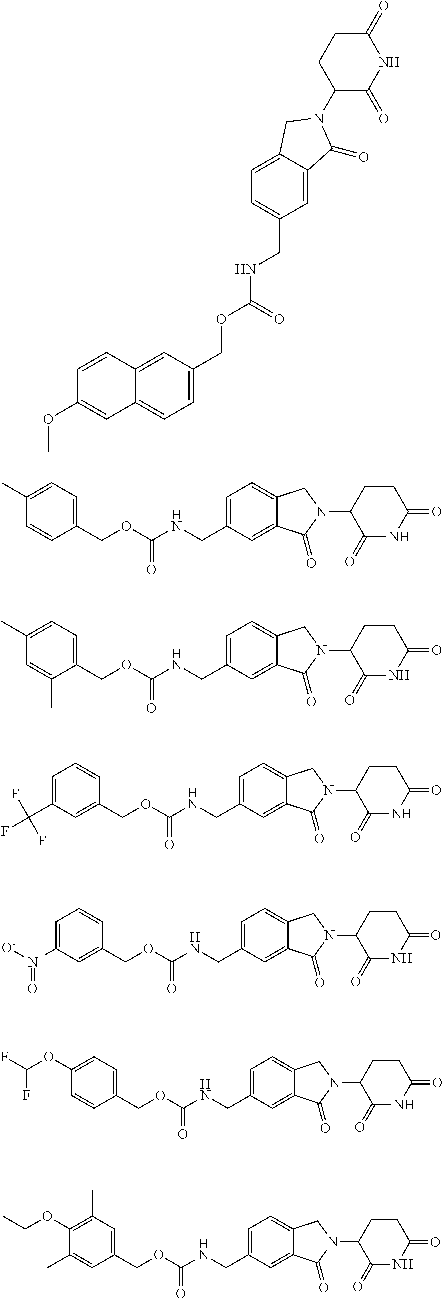 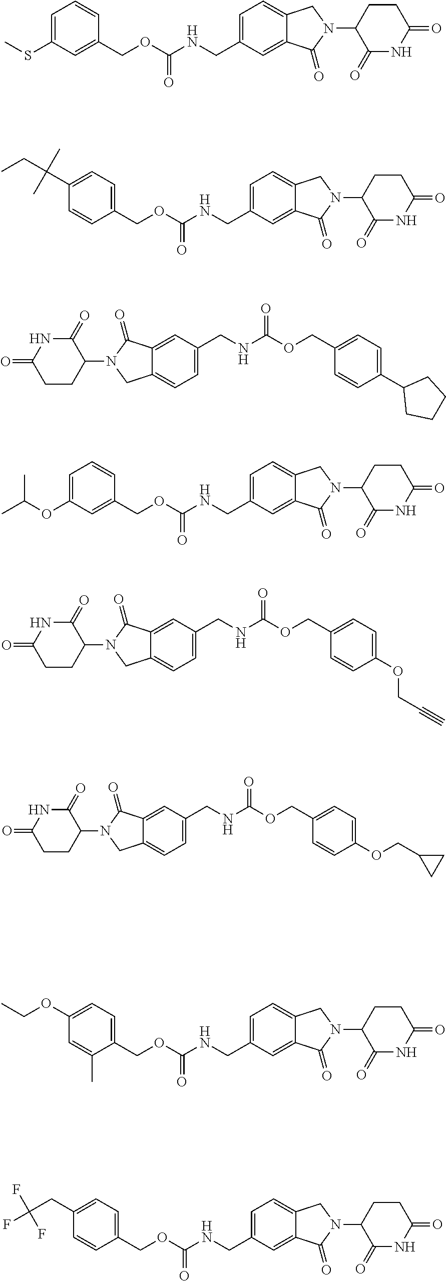 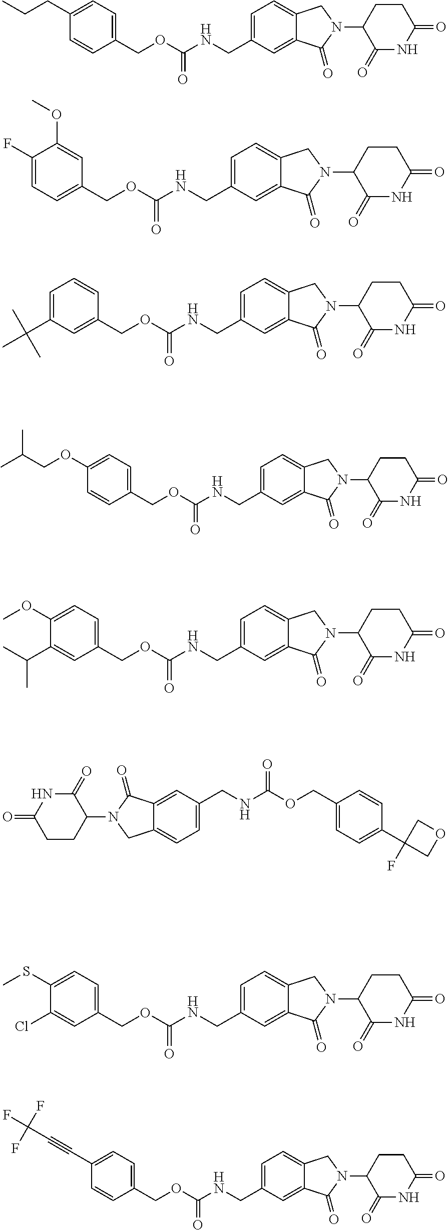 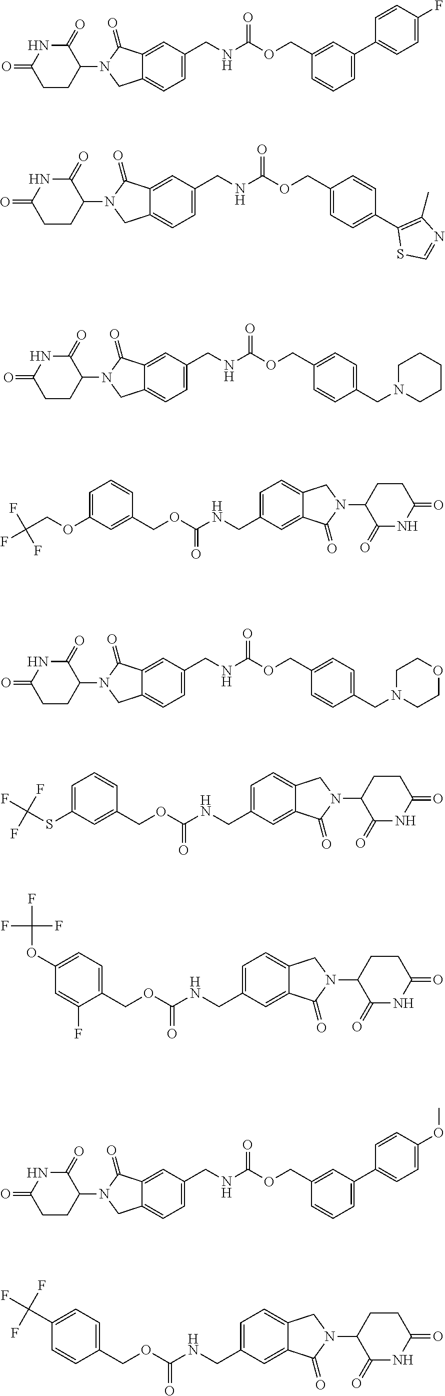 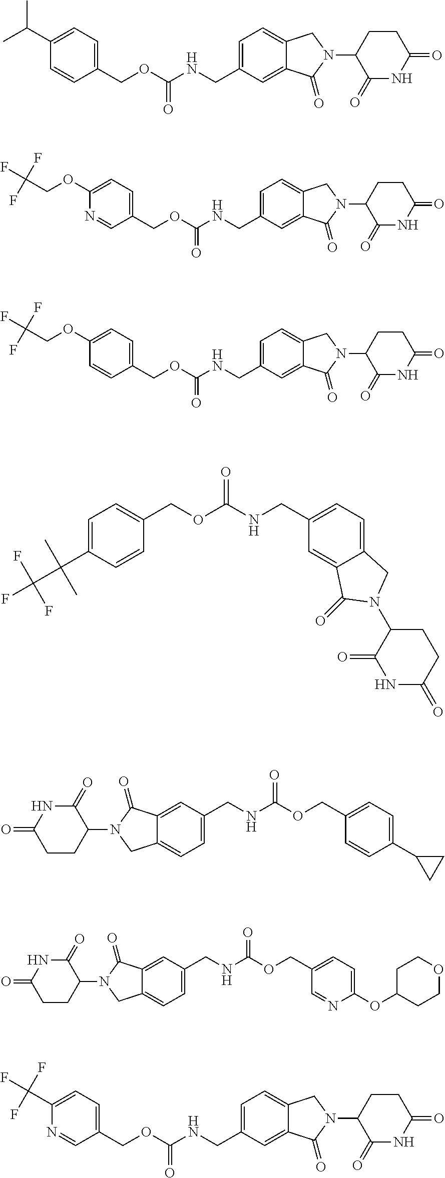 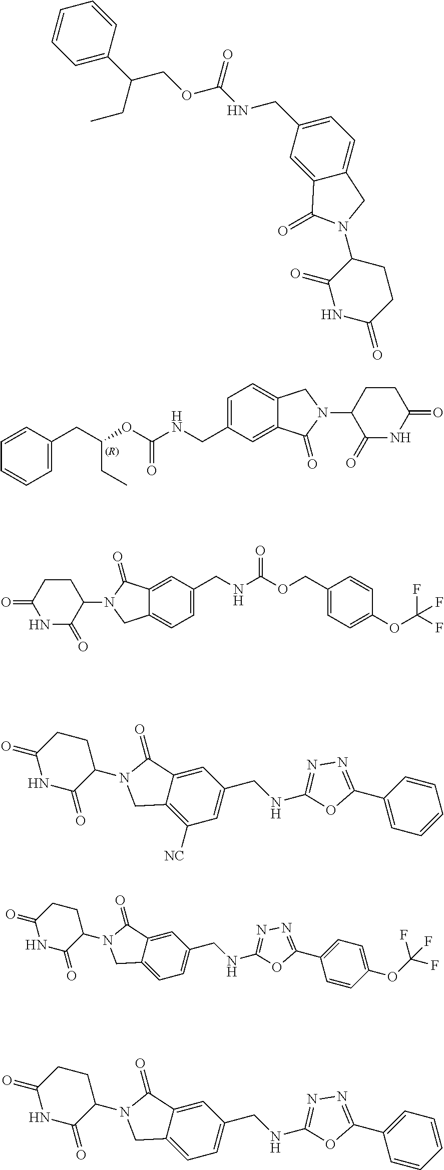 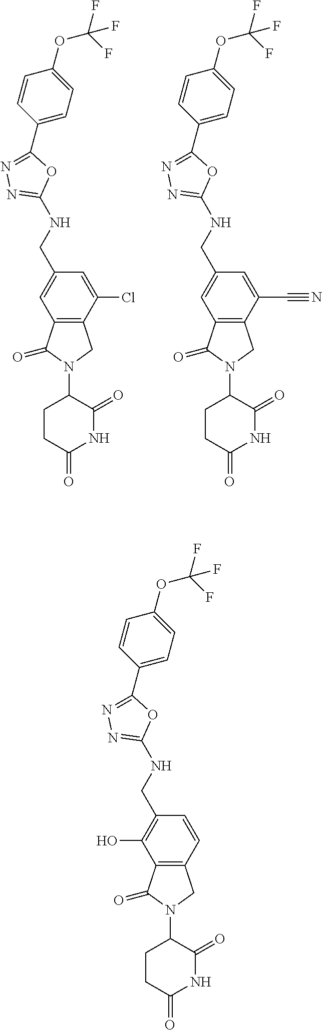 [SMILES: O=C1CCC(N2Cc3c(Cl)cc(CNc4nnc(-c5ccc(OC(F)(F)F)cc5)o4)cc3C2=O)C(=O)N1 ; N#Cc1cc(CNc2nnc(-c3ccc(OC(F)(F)F)cc3)o2)cc2c1CN(C1CCC(=O)NC1=O)C2=O ; O=C1CCC(N2Cc3ccc(CNc4nnc(-c5ccc(OC(F)(F)F)cc5)o4)c(O)c3C2=O)C(=O)N1]
and pharmaceutically acceptable salts thereof.

17. A pharmaceutical composition comprising the compound of claim 1, or pharmaceutically acceptable salt thereof, and a pharmaceutically acceptable excipient.

18. A pharmaceutical composition comprising the compound of claim 16, or pharmaceutically acceptable salt thereof, and a pharmaceutically acceptable excipient.

19. A method of degrading CDK2 in a subject suffering from cancer, comprising administering to the subject an effective amount of the compound of claim 1.

20. A method of treating cancer in a subject in need thereof, comprising administering to the subject a therapeutically effective amount of the compound of claim 1.
